(19) United States
(12) Patent Application Publication
(10) Pub. No.: US 2024/0384312 A1
(43) Pub. Date: Nov. 21, 2024

(54) ENZYMES, MICRO-ORGANISMS AND USES THEREOF, AND A METHOD OF DEGRADING HYDROCARBON CHAINS

(71) Applicant: TEKNOLOGIAN TUTKIMUSKESKUS VTT OY, Espoo (FI)

(72) Inventors: Kari KOIVURANTA, Espoo (FI); Sandra CASTILLO, Espoo (FI); Heli NYGREN, Espoo (FI)

(21) Appl. No.: 18/579,070

(22) PCT Filed: Jul. 12, 2022
(86) PCT No.: PCT/FI2022/050495

§ 371 Date: Jan. 12, 2024

(30) Foreign Application Priority Data
Jul. 14, 2021 (FI) 20215805

(51) Int. Cl. C12P 7/625; C12N 9/02; C12N 9/08

(52) U.S. Cl. CPC C12P 7/625; C12N 9/0065; C12N 9/0089; C12Y 111/0101; C12Y 115/01001

(57) ABSTRACT

The present invention relates to the fields of life sciences, micro-organisms and degradation of hydrocarbon chains such as polyolefins. Specifically, the invention relates to an isolated specific enzyme or a fragment thereof, wherein said enzyme or fragment is a superoxide dismutase and is capable of degrading a hydrocarbon chain or a polyolefin, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a hydrocarbon chain such as a polyolefin; to a method of degrading a hydrocarbon chain such as a polyolefin with the specific superoxide dismutase enzyme or a fragment thereof; and to a method of producing the enzyme or fragment thereof of the present invention.

### FIELD OF THE INVENTION

The present invention relates to the fields of life sciences, micro-organisms and degradation of hydrocarbon chains such as polyolefins. Specifically, the invention relates to an isolated specific enzyme or a fragment thereof, wherein said enzyme or fragment is capable of degrading a hydrocarbon chain such as a polyolefin, and to a micro-organism or a host cell comprising the enzyme or a fragment thereof. Also, the present invention relates to a polynucleotide encoding the enzyme or fragment thereof, and to an expression vector or plasmid comprising the polynucleotide of the present invention. And still, the present invention relates to use of the enzyme, fragment, micro-organism, host cell, polynucleotide, expression vector or plasmid of the present invention for degrading a hydrocarbon chain such as a polyolefin; to a method of degrading a hydrocarbon chain such as a polyolefin with the specific enzyme or a fragment thereof; and to a method of producing the enzyme or fragment thereof of the present invention. Further, the present invention relates to a method of producing fatty acid derived products such as hydroxy fatty acids and/or polyhydroxyalkanoate (PHA) from the degradation products of hydrocarbons, such as polyolefins, by the enzymes, micro-organisms and/or host cells of the present invention.

### BACKGROUND OF THE INVENTION

With the existing plastic recycling systems (mechanical and chemical) not all plastic waste can be recycled. This is partly due to the quality of plastic wastes (mixed plastic, dirty plastics). Additionally, the existing recycling methods need much energy. Biotechnical recycling could be utilized for improving the range of recycling methods and for enabling cost effective and more efficient recycling of plastics.

Removal of highly stable and durable hydrocarbon chains such as polyolefin polymers including but not limited to plastics comprising polyolefins from the environment by using microbes or microbial enzymes is of high interest. In general, biotechnical plastic degradation is not common yet. Only few micro-organisms or enzymes capable of degrading polyolefins have been discovered and said micro-organisms or enzymes are not effective. For example, Santo M. et al. (2013, International Biodeterioration & Biodegradation 84, 204-210) describe degradation of polyethylene (PE) with an extracellular fraction comprising different enzymes obtained from a Rhodococcus ruber cell culture. However, for PE or other polyolefins, recycling systems utilizing specific enzymes including but not limited to isolated and/or purified enzymes are under development. Indeed, it is very difficult to degrade hydrocarbon chains with enzymes.

Micro-organisms and enzymes are needed for rapid degradation and recycling of hydrocarbon chains. There remains a significant unmet need for specific micro-organisms and enzymes for effective degradation of hydrocarbon chains such as polyolefin polymers or plastics.

### BRIEF DESCRIPTION OF THE INVENTION

By biotechnical degradation and tools of the present invention it is possible to degrade and therefore recycle hydrocarbon chains such as plastics or synthetic polymers and more specifically polyolefins. Furthermore, the tools of the present invention can be used e.g., for upcycling hydrocarbon chains i.e., for modifying a non-biodegradable plastic or polyolefin (e.g., PE) to a biodegradable plastic (such as polyhydroxyalkanoate (PHA)) or fatty acid derived products (such as PHA, hydroxy fatty acids and/or diacids) by micro-organisms and enzymes.

The objects of the invention, namely methods and tools for degrading hydrocarbon chains such as polyolefins are achieved by utilizing a specific enzyme or enzymes, or a specific micro-organism or micro-organisms (e.g., a bacterium/bacteria and/or fungus/fungi) comprising said enzyme(s).

The present invention provides methods and tools which enable biotechnical degradation of hydrocarbon chains or polyolefins. Said methods and tools provide surprising degradation effects on hydrocarbon chains such as polyolefins or on a combination of specific plastics or polymers comprising polyolefins. Also, the present invention can overcome the problems of the prior art including but not limited to ineffective or slow biotechnical degradation of hydrocarbon chains or polyolefin polymers. Furthermore, the specific enzyme or micro-organism of the present invention enable degradation methods at low temperatures, e.g., at a temperature below 100° C., indicating low energy need and costs.

Also, the inventors of the present disclosure surprisingly found out that unique or specific degradation products can be obtained with the present invention.

Further, the inventors found that fatty acid derived products such as hydroxy fatty acids and/or polyhydroxyalkanoate (PHA) can be produced from the degradation product(s) of hydrocarbon chains by the enzymes, micro-organisms and/or host cells of the present invention as substrates for an enzyme, a micro-organism and/or a host cell producing hydroxy fatty acids and/or polyhydroxyalkanoate (PHA).

Specifically, the present invention relates to a method of degrading a hydrocarbon chain or a polyolefin, said method comprising
providing a material comprising a hydrocarbon chain or a polyolefin and an enzyme or a fragment thereof capable of degrading the hydrocarbon chain or the polyolefin, and
allowing said enzyme or fragment thereof to degrade the hydrocarbon chain or the polyolefin, wherein the enzyme or fragment thereof comprises one or more amino acids selected from the group comprising Leu15, Pro17, His27, His31, His32, Tyr35, Asn40, His82, Trp86, Ile105, Glyl28, Ser129, Asp164, Trp166, Glu167, His168, Tyr171, and Asn176 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10.

In one embodiment, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises one or more amino acids selected from the group comprising His27 His31, His32, Tyr35, Asn40, Trp166, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10.

In one embodiment, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises one or more amino acids selected from the group comprising His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10.

In one embodiment, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises the amino acids His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10.

In one embodiment, the enzyme or fragment thereof degrading the hydrocarbon chain, comprises the amino acids His31, Tyr35, Glu167, His168 and Tyr171 and one or more amino acids selected from the group comprising Leu15, Pro17, His27, His32, Asn40, His82, Trp86, Ile105, Glyl28, Ser129, Asp164, Trp166, and Asn176 corresponding to the amino acid positions presented in SEQ ID NO: 2.

In one embodiment, the enzyme or fragment thereof degrading the hydrocarbon chain, comprises the amino acids His31, Tyr35, Glu167, His168 and Tyr171 and one or more amino acids selected from the group comprising His27, His32, Asn40, and Trp166 corresponding to the amino acid positions presented in SEQ ID NO: 2.

In one embodiment, the present invention relates to a method of degrading a hydrocarbon chain, said method comprising
providing a material comprising a hydrocarbon chain and an enzyme or a fragment thereof capable of degrading the hydrocarbon chain, and
allowing said enzyme or fragment thereof to degrade the hydrocarbon chain, wherein the enzyme or fragment thereof comprises the amino acids His27 His31, His32, Tyr35, Asn40, Trp166, Glu167, His168 and Tyr171, corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10.

In one embodiment, the enzyme or fragment thereof degrading the hydrocarbon chain, comprises the amino acids His27 His31, His32, Tyr35, Asn40, Trp166, Glu167, His168, and Tyr171, and one or more amino acids selected from the group comprising Leu15, Pro17, His82, Trp86, Ile105, Glyl28, Ser129, Asp164 and Asn176 corresponding to the amino acid positions presented in SEQ ID NO: 2.

Also, the present invention relates to an isolated enzyme or a fragment thereof comprising one or more amino acids selected from the group comprising Leu15, Pro17, His27, His31, His32, Tyr35, Asn40, His82, Trp86, Ile105, Glyl28, Ser129, Asp164, Trp166, Glu167, His168, Tyr171 and Asn176 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the present invention relates to an isolated enzyme or a fragment thereof comprising one or more amino acids selected from the group comprising His27 His31, His32, Tyr35, Asn40, Trp166, Glu167, His168, and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the present invention relates an isolated enzyme or a fragment thereof comprising one or more amino acids selected from the group comprising His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the present invention relates to an isolated enzyme or a fragment thereof comprising the amino acids His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the enzyme or fragment thereof degrading the hydrocarbon chain, comprises the amino acids His31, Tyr35, Glu167, His168 and Tyr171 and one or more amino acids selected from the group comprising Leu15, Pro17, His27, His32, Asn40, His82, Trp86, Ile105, Glyl28, Ser129, Asp164, Trp166, and Asn176 corresponding to the amino acid positions presented in SEQ ID NO: 2.

In one embodiment, the enzyme or fragment thereof degrading the hydrocarbon chain, comprises the amino acids His31, Tyr35, Glu167, His168 and Tyr171 and one or more amino acids selected from the group comprising His27, His32, Asn40, and Trp166 corresponding to the amino acid positions presented in SEQ ID NO: 2.

In one embodiment, the present invention relates to an isolated enzyme or a fragment thereof comprising the amino acids His27 His31, His32, Tyr35, Asn40, Trp166, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the enzyme or fragment thereof degrading the hydrocarbon chain, comprises the amino acids His27 His31, His32, Tyr35, Asn40, Trp166, Glu167, His168, and Tyr171, and one or more amino acids selected from the group comprising Leu15, Pro17, His82, Trp86, Ile105, Glyl28, Ser129, Asp164 and Asn176 corresponding to the amino acid positions presented in SEQ ID NO: 2.

Furthermore, the present invention relates to a micro-organism or a host cell comprising an enzyme or a fragment thereof comprising one or more amino acids selected from the group comprising Leu15, Pro17, His27, His31, His32, Tyr35, Asn40, His82, Trp86, Ile105, Glyl28, Ser129, Asp164, Trp166, Glu167, His168, Tyr171, and Asn176 corresponding to the amino acid positions presented in SEQ ID NO: 2, and/or having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8 or 10, wherein said enzyme or fragment is capable of degrading a hydrocarbon chain.

In one embodiment, the present invention relates to a micro-organism or a host cell comprising an isolated enzyme or a fragment thereof comprising one or more amino acids selected from the group comprising His27 His31, His32, Tyr35, Asn40, Trp166, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the present invention relates to a micro-organism or a host cell comprising an isolated enzyme or a fragment thereof comprising one or more amino acids selected from the group comprising His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the present invention relates to a micro-organism or a host cell comprising an isolated enzyme or a fragment thereof comprising the amino acids His31, Tyr35, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the enzyme or fragment thereof degrading the hydrocarbon chain, comprises the amino acids His31, Tyr35, Glu167, His168 and Tyr171 and one or more amino acids selected from the group comprising Leu15, Pro17, His27, His32, Asn40, His82, Trp86, Ile105, Glyl28, Ser129, Asp164, Trp166, and Asn176 corresponding to the amino acid positions presented in SEQ ID NO: 2.

In one embodiment, the enzyme or fragment thereof degrading the hydrocarbon chain, comprises the amino acids His31, Tyr35, Glu167, His168 and Tyr171 and one or more amino acids selected from the group comprising His27, His32, Asn40, and Trp166 corresponding to the amino acid positions presented in SEQ ID NO: 2.

In one embodiment, the present invention relates to a micro-organism or a host cell comprising an isolated enzyme or a fragment thereof comprising the amino acids His27 His31, His32, Tyr35, Asn40, Trp166, Glu167, His168 and Tyr171 corresponding to the amino acid positions presented in SEQ ID NO: 2, and/or the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain.

In one embodiment, the enzyme or fragment thereof degrading the hydrocarbon chain, comprises the amino acids His27 His31, His32, Tyr35, Asn40, Trp166, Glu167, His168, and Tyr171, and one or more amino acids selected from the group comprising Leu15, Pro17, His82, Trp86, Ile105, Glyl28, Ser129, Asp164 and Asn176 corresponding to the amino acid positions presented in SEQ ID NO: 2.

Still, the present invention relates to a polynucleotide encoding the enzyme or fragment thereof of the present invention.

Still, the present invention relates to an expression vector or plasmid comprising the polynucleotide of the present invention.

And still, the present invention relates to use of the enzyme, fragment, micro-organism, host cell, polynucleotide, expression vector or plasmid of the present invention or any combination thereof for degrading a hydrocarbon chain or a polyolefin.

Still furthermore, the present invention relates to a method of producing the enzyme or fragment thereof of the present invention, wherein a recombinant micro-organism or host cell comprising the polynucleotide encoding the enzyme or fragment thereof of the present invention is allowed to express said enzyme or fragment thereof.

The present invention also relates to a method of producing fatty acid derived products such as hydroxy fatty acids and/or diacids, and/or polyhydroxyalkanoate (PHA) from the degradation products of hydrocarbons by the enzymes, micro-organisms and/host cells of the present invention as substrates for an enzyme, a micro-organism and/or a host cell producing diacids, hydroxy fatty acids and/or polyhydroxyalkanoate (PHA).

Other objects, details and advantages of the present invention will become apparent from the following drawings, detailed description, and examples.

### BRIEF DESCRIPTION OF THE DRAWINGS

FIG. 1 shows results from the GC-MS run. With Bacillus licheniformis, Bacillus cereus, Bacillus flexus, Bacillus subtilis and Rhodococcus ruber enzyme samples several peaks appeared which were missing from control sample (in controls an empty plasmid) with polypropylene powder.

FIG. 2 shows results from the GC-MS run. With Bacillus flexus and Rhodococcus ruber enzyme samples several peaks appeared which were missing from control samples (in control an empty plasmid) with polyethylene powder.

FIG. 3 shows results from the GC-MS run. With Bacillus licheniformis, Bacillus cereus and Bacillus subtilis enzyme samples several peaks appeared which were not seen in control sample (in control an empty plasmid) with polyethylene powder.

FIG. 4 shows an alignment of several amino acid sequences of micro-organisms and consensus amino acids based on Bacillus licheniformis superoxide dismutase amino acid positions Leu15, Pro17, His27, His31, His32, Tyr35, Asn40, His82, Trp86, Ile105, Glyl28, Ser129, Asp164, Trp166, Glu167, His168, Tyr171, Asn176.

FIG. 5 shows a pairwise alignment of Streptomyces badius superoxide dismutase (SEQ ID NO: 113) and B. licheniformis superoxide dismutase (SEQ ID NO: 2). Detected consensus amino acids have been marked with bold in Streptomyces badius amino acid sequence.

FIG. 6 shows two-dimensional structure (alfa helixes and beta sheets) of Bacillus licheniformis superoxide dismutase (SEQ ID NO: 2) and localisation of consensus amino acids. Alfa helixes are underlined and numbered with Arabic numbers. Beta sheets are in Italics and numbered with Roman numbers. Consensus amino acids are in bold.

FIG. 7 shows results from the GC-MS run. Purified Bacillus licheniformis superoxide dismutase was incubated with octadecane and degrease in octadecane amount could be detected which was not seen in a control reaction which was carried out without enzyme.

FIG. 8 shows results from the GC-MS run. Purified Bacillus licheniformis superoxide dismutase was incubated with octadecanoic acid and degrease in octadecanoic acid amount could be detected which was not seen in a control reaction which was carried out without enzyme.

FIG. 9 shows a plasmid map of pPB098-1.

FIG. 10 shows a plasmid map of pPB113.

FIG. 11 shows results from the GC-MS run. With Yarrowia lipolytica strain expressing Bacillus flexus superoxide dismutase and Pseudomonas sp. PHA synthase hydroxy fatty acids could be detected in PE cultivation which were not seen with the wild type Yarrowia lipolytica.

FIG. 12 shows results from the GC-MS run. With Yarrowia lipolytica strain expressing Bacillus flexus superoxide dismutase and Pseudomonas sp. PHA synthase hydroxy fatty acids could be detected in PE cultivation (PE sample) which were not seen in the cultivations without PE (Y and C samples).

FIG. 13 shows the plasmid map of pPB111.

### SEQUENCE LISTING

SEQ ID NO: 1: Bacillus licheniformis superoxide dismutase nucleotide sequence;
SEQ ID NO: 2: Bacillus licheniformis superoxide dismutase amino acid sequence;
SEQ ID NO: 3: Bacillus cereus superoxide dismutase nucleotide sequence;
SEQ ID NO: 4: Bacillus cereus superoxide dismutase amino acid sequence;
SEQ ID NO: 5: Bacillus flexus superoxide dismutase nucleotide sequence;
SEQ ID NO: 6: Bacillus flexus superoxide dismutase amino acid sequence;
SEQ ID NO: 7: Bacillus subtilis superoxide dismutase nucleotide sequence;
SEQ ID NO: 8: Bacillus subtilis superoxide dismutase amino acid sequence;
SEQ ID NO: 9: Rhodococcus ruber superoxide dismutase nucleotide sequence;
SEQ ID NO: 10: Rhodococcus ruber superoxide dismutase amino acid sequence;
SEQ ID NO: 11: oPlastBug-242 oligonucleotide;
SEQ ID NO: 12: oPlastBug-243 oligonucleotide;
SEQ ID NO: 13: oPlastBug-238 oligonucleotide;
SEQ ID NO: 14: oPlastBug-239 oligonucleotide;
SEQ ID NO: 15: oPlastBug-138 oligonucleotide;
SEQ ID NO: 16: oPlastBug-139 oligonucleotide;
SEQ ID NO: 17: oPlastBug-244 oligonucleotide;
SEQ ID NO: 18: oPlastBug-245 oligonucleotide;
SEQ ID NO: 19: oPlastBug-136 oligonucleotide;
SEQ ID NO: 20: oPlastBug-137 oligonucleotide;
SEQ ID NO: 21: Bacillus flexus superoxide dismutase amino acid sequence with Ser83Asn and Ser115Lys mutations and with Yarrowia lipolytica LIP2 signal peptide;
SEQ ID NO: 22: Nucleotide sequence of Bacillus flexus superoxide dismutase with Ser51Asn and Ser83Lys mutations and with Yarrowia lipolytica LIP2 signal peptide codon optimised to Yarrowia lipolytica;
SEQ ID NO:23: Bacillus cohnii superoxide dismutase amino acid sequence;
SEQ ID NO: 24: Achromobacter xylosoxidans superoxide dismutase amino acid sequence;
SEQ ID NO: 25: Acinetobacter baumannii superoxide dismutase amino acid sequence;
SEQ ID NO: 26: Acinetobacter pittii superoxide dismutase amino acid sequence;
SEQ ID NO: 27: Alcanivorax borkumensis superoxide dismutase amino acid sequence;
SEQ ID NO: 28: Aneurinibacillus aneurinilyticus superoxide dismutase amino acid sequence;
SEQ ID NO: 29: Arthobacter sp. superoxide dismutase amino acid sequence;
SEQ ID NO: 30: Aspergillus awamori superoxide dismutase amino acid sequence;
SEQ ID NO: 31: Aspergillus flavus superoxide dismutase amino acid sequence;
SEQ ID NO: 32: Aspergillus fumigatus superoxide dismutase amino acid sequence;
SEQ ID NO: 33: Aspergillus glaucus superoxide dismutase amino acid sequence;
SEQ ID NO: 34: Aspergillus niger superoxide dismutase amino acid sequence;
SEQ ID NO: 35: Aspergillus oryzae superoxide dismutase amino acid sequence;
SEQ ID NO: 36: Aspergillus sp. superoxide dismutase amino acid sequence;
SEQ ID NO: 37: Aspergillus sydowii superoxide dismutase amino acid sequence;
SEQ ID NO: 38: Aspergillus terreus superoxide dismutase amino acid sequence;
SEQ ID NO: 39: Bacillus sp. superoxide dismutase amino acid sequence;
SEQ ID NO: 40: Bacillus amyloliquefaciens superoxide dismutase amino acid sequence;
SEQ ID NO: 41: Bacillus aryabhattai superoxide dismutase amino acid sequence;
SEQ ID NO: 42: Bacillus mycoides superoxide dismutase amino acid sequence;
SEQ ID NO: 43: Bacillus pumilus superoxide dismutase amino acid sequence;
SEQ ID NO: 44: Bacillus thuringiensis superoxide dismutase amino acid sequence;
SEQ ID NO: 45: Bacillus vallismortis superoxide dismutase amino acid sequence;
SEQ ID NO: 46: Bacillus vietnamensis superoxide dismutase amino acid sequence;
SEQ ID NO: 47: Brevibacillus agri superoxide dismutase amino acid sequence;
SEQ ID NO: 48: Brevibacillus borstelensis superoxide dismutase amino acid sequence;
SEQ ID NO: 49: Brevibacillus brevis superoxide dismutase amino acid sequence;
SEQ ID NO: 50: Brevibacillus parabrevis superoxide dismutase amino acid sequence;
SEQ ID NO: 51: Brevibacillus sp. superoxide dismutase amino acid sequence;
SEQ ID NO: 52: Citrobacter amalonaticus superoxide dismutase amino acid sequence;
SEQ ID NO: 53: Comamonas sp. superoxide dismutase amino acid sequence;
SEQ ID NO: 54: Cordyceps confragosa. superoxide dismutase amino acid sequence;
SEQ ID NO: 55: Cupriavidus necator superoxide dismutase amino acid sequence;
SEQ ID NO: 56: Delftia sp. superoxide dismutase amino acid sequence;
SEQ ID NO: 57: Delftia tsuruhatensis superoxide dismutase amino acid sequence;
SEQ ID NO: 58: Enterobacter sp. superoxide dismutase amino acid sequence;
SEQ ID NO: 59: Enterobacter asburiae superoxide dismutase amino acid sequence;
SEQ ID NO: 60: Escherichia coli superoxide dismutase amino acid sequence;
SEQ ID NO: 61: Flavobacterium sp. superoxide dismutase amino acid sequence;
SEQ ID NO: 62: Fusarium solani superoxide dismutase amino acid sequence;
SEQ ID NO: 63: Fusarium sp. superoxide dismutase amino acid sequence;
SEQ ID NO: 64: Klebsiella pneumoniae superoxide dismutase amino acid sequence;
SEQ ID NO: 65: Kocuria palustris superoxide dismutase amino acid sequence;
SEQ ID NO: 66: Leucobacter sp. superoxide dismutase amino acid sequence;
SEQ ID NO: 67: Lysinibacillus fusiformis superoxide dismutase amino acid sequence;
SEQ ID NO: 68: Lysinibacillus sphaericus superoxide dismutase amino acid sequence;
SEQ ID NO: 69: Lysinibacillus xylanilyticus superoxide dismutase amino acid sequence;
SEQ ID NO: 70: Microbacterium paraoxydans superoxide dismutase amino acid sequence;
SEQ ID NO: 71: Micrococcus luteus superoxide dismutase amino acid sequence;
SEQ ID NO: 72: Micrococcus lylae superoxide dismutase amino acid sequence;
SEQ ID NO: 73: Micrococcus sp. superoxide dismutase amino acid sequence;
SEQ ID NO: 74: Moraxella sp. superoxide dismutase amino acid sequence;
SEQ ID NO: 75: Mucor circinelloides superoxide dismutase amino acid sequence;
SEQ ID NO: 76: Nesiobacter exalbescens superoxide dismutase amino acid sequence;
SEQ ID NO: 77: Nocardia asteroides superoxide dismutase amino acid sequence;
SEQ ID NO: 78: Ochrobactrum intermedium superoxide dismutase amino acid sequence;
SEQ ID NO: 79: Ochrobactrum oryzae superoxide dismutase amino acid sequence;
SEQ ID NO: 80: Paenibacillus macerans superoxide dismutase amino acid sequence;
SEQ ID NO: 81: Paenibacillus sp. superoxide dismutase amino acid sequence;
SEQ ID NO: 82: Pantoea sp. superoxide dismutase amino acid sequence;
SEQ ID NO: 83: Penicillium chrysogenum superoxide dismutase amino acid sequence;
SEQ ID NO: 84: Penicillium oxalicum superoxide dismutase amino acid sequence;
SEQ ID NO: 85: Pleurotus ostreatus superoxide dismutase amino acid sequence;
SEQ ID NO: 86: Pseudomonas aeruginosa superoxide dismutase amino acid sequence;
SEQ ID NO: 87: Pseudomonas azotoformans superoxide dismutase amino acid sequence;
SEQ ID NO: 88: Pseudomonas chlororaphis superoxide dismutase amino acid sequence;
SEQ ID NO: 89: Pseudomonas citronellolis superoxide dismutase amino acid sequence;
SEQ ID NO: 90: Pseudomonas fluorescens superoxide dismutase amino acid sequence;
SEQ ID NO: 91: Pseudomonas monteilii superoxide dismutase amino acid sequence;
SEQ ID NO: 92: Pseudomonas protegens superoxide dismutase amino acid sequence;
SEQ ID NO: 93: Pseudomonas putida superoxide dismutase amino acid sequence;
SEQ ID NO: 94: Pseudomonas sp. superoxide dismutase amino acid sequence;
SEQ ID NO: 95: Pseudomonas stutzeri superoxide dismutase amino acid sequence;
SEQ ID NO: 96: Pseudomonas syringae superoxide dismutase amino acid sequence;
SEQ ID NO: 97: Rahnella aquatilis superoxide dismutase amino acid sequence;
SEQ ID NO: 98: Ralstonia sp. superoxide dismutase amino acid sequence;
SEQ ID NO: 99: Rhodococcus erythropolis superoxide dismutase amino acid sequence;
SEQ ID NO: 100: Rhodococcus rhodochrous superoxide dismutase amino acid sequence;
SEQ ID NO: 101: Rhodococcus sp. superoxide dismutase amino acid sequence;
SEQ ID NO: 102: Serratia marcescens superoxide dismutase amino acid sequence;
SEQ ID NO: 103: Sphingobacterium multivorum superoxide dismutase amino acid sequence;
SEQ ID NO: 104: Staphylococcus cohnii superoxide dismutase amino acid sequence;
SEQ ID NO: 105: Staphylococcus epidermidis superoxide dismutase amino acid sequence;
SEQ ID NO: 106: Staphylococcus sp. superoxide dismutase amino acid sequence;
SEQ ID NO: 107: Staphylococcus xylosus superoxide dismutase amino acid sequence;
SEQ ID NO: 108: Stenotrophomonas humi superoxide dismutase amino acid sequence;
SEQ ID NO: 109: Stenotrophomonas maltophila superoxide dismutase amino acid sequence;
SEQ ID NO: 110: Stenotrophomonas panacihumi superoxide dismutase amino acid sequence;
SEQ ID NO: 111: Stenotrophomonas sp. superoxide dismutase amino acid sequence;
SEQ ID NO: 112: Streptococcus sp. superoxide dismutase amino acid sequence;
SEQ ID NO: 113: Streptomyces badius superoxide dismutase amino acid sequence;
SEQ ID NO: 114: Streptomyces griseus superoxide dismutase amino acid sequence;
SEQ ID NO: 115: Streptomyces sp. superoxide dismutase amino acid sequence;
SEQ ID NO: 116: Trichoderma harzianum superoxide dismutase amino acid sequence;
SEQ ID NO: 117: Trichoderma virens superoxide dismutase amino acid sequence;
SEQ ID NO: 118: Vibrio alginolyticus superoxide dismutase amino acid sequence;
SEQ ID NO: 119: Vibrio parahaemolyticus superoxide dismutase amino acid sequence;
SEQ ID NO: 120: Brucella anthropi superoxide dismutase 1 amino acid sequence;
SEQ ID NO: 121: Brucella anthropi superoxide dismutase 2 amino acid sequence;
SEQ ID NO: 122: Halomonas venusta superoxide dismutase amino acid sequence;
SEQ ID NO: 123: Exiguobacterium sp. superoxide dismutase amino acid sequence;
SEQ ID NO: 124: Pseudomonas sp. PHA synthase amino acid sequence;
SEQ ID NO: 125: Nucleotide sequence of Pseudomonas sp. PHA synthase codon optimised to Yarrowia lipolytica;
SEQ ID NO: 126: oPlastBug-268 oligonucleotide;
SEQ ID NO: 127: oPlastBug-270 oligonucleotide.
SEQ ID NO: 128: oPlastBug-266 oligonucleotide;
SEQ ID NO: 129: oPlastBug-267 oligonucleotide;
SEQ ID NO: 130: Bacillus cereus chloroperoxidase amino acid sequence;
SEQ ID NO: 131: Bacillus cereus chloroperoxidase nucleotide sequence codon optimised to Yarrowia lipolytica.

### DETAILED DESCRIPTION OF THE INVENTION

The present invention concerns a method of degrading a hydrocarbon chain such as a polyolefin, for example, wherein a specific enzyme or micro-organism of the present invention is used for degrading said hydrocarbon chain. In one embodiment of the present invention a hydrocarbon chain or a polyolefin or a material comprising one or more hydrocarbon chains or polyolefins or types of polyolefins (such as plastics or polymers of fossil origin, bio-based polymers or plastic material, polymer composites, copolymers, packaging material, textile, plastics or synthetic polymers (e.g. oil-based and/or biobased) containing waste material) is allowed to contact with an enzyme or micro-organism capable of degrading the hydrocarbon chain(s) or polyolefin(s). In one embodiment of the invention the material comprising one or more hydrocarbon chains, one or more polyolefins or types of polyolefins is a recycled material or from a recycled material.

As used herein, “a plastic” refers to a material comprising or consisting of synthetic and/or semi-synthetic organic compounds and having the capability of being molded or shaped. As used herein “a synthetic polymer” refers to a human-made polymer. Synthetic polymers can be classified into four main categories: thermoplastics, thermosets, elastomers, and synthetic fibers. Thermoplastics are a type of synthetic polymers that become moldable and malleable past a certain temperature, and they solidify upon cooling. Thermosets become hard and cannot change shape once they have set. Elastomers are flexible polymers. Synthetic fibers are fibers made by humans through a chemical synthesis.

As used herein “a hydrocarbon chain” refers to an organic compound, which comprises or consists of a chain of hydrogens and carbons (e.g., at least 4C). In one embodiment the chain of hydrogens and carbons is linear, acyclic, cyclic, branched, aliphatic and/or aromatic. Therefore, “a hydrocarbon chain” refers e.g. to a hydrocarbon or a chain comprising a hydrocarbon chain like structure e.g. in the other end or one end of the chain. For example, long alkanes, alkenes, fatty acids, alcohols, aldehydes and ketones (e.g., comprising at least 10C hydrocarbon chain like structure in the other end of the chain) and other compounds comprising a long hydrocarbon chain (e.g., at least 10C hydrocarbon) like structure are within the scope of “hydrocarbon chains”. Compounds comprising at least one long hydrocarbon chain (e.g., at least 10C hydrocarbon) like structure can have been obtained e.g., by a polymerization reaction.

Hydrocarbons can be classified to saturated hydrocarbons, unsaturated hydrocarbons, and aromatic hydrocarbons. Saturated hydrocarbons comprise single bonds and are saturated with hydrogen. The formula for acyclic saturated hydrocarbons (i.e., alkanes) is CₙH₂ₙ₊₂. The most general form of saturated hydrocarbons is CₙH₂ₙ₊₂₍₁₋ᵣ₎, wherein r is the number of rings. Unsaturated hydrocarbons have one or more double or triple bonds between carbon atoms. Unsaturated hydrocarbons with double bonds are called alkenes and unsaturated hydrocarbons comprising triple bonds are called alkynes. Those with one double bond have the formula CₙH₂ₙ (assuming non-cyclic structures). Those with one triple bond have the formula CₙH₂ₙ₋₂. Aromatic hydrocarbons (arenes) have at least one aromatic ring.

In one embodiment a hydrocarbon chain (e.g., a linear hydrocarbon chain) is selected from the group comprising or consisting of polymers (e.g., plastics such as polyethylene, polypropylene, polystyrene, or multilayer materials or mixtures of materials comprising synthetic polymers or plastics and furthermore one or more materials such as paper and/or cardboard); gases (e.g., 1,7-octadiene); and liquids (e.g., dodecane). In one embodiment a hydrocarbon chain (e.g., a chain or compound comprising a hydrocarbon like structure) is selected from the group comprising or consisting of a long ketone, long alkane, long alkene, long alkyne, long cycloalkane, long alkadiene, long fatty acid, long alcohol and long carbon chain aldehyde. In one embodiment, a long alkane or a long fatty acid has 15-25 carbon atoms or 18-25 carbon atoms, for example. In one embodiment, a long alkane or a long fatty acid has at least 15 carbon atoms, at least 18 carbon atoms or at least 25 carbon atoms.

As used herein polyolefin refers to a type of polymer produced from a simple olefin (e.g., called an alkene with the general formula CₙH₂n) as a monomer. For example, polyethylene and polypropylene are common polyolefins. Depending on a polymerization method utilized for producing a polyolefin, the polyolefin hydrocarbon chain can sometimes comprise a specific group or groups such as a ketone group e.g. at the end of the chain. Polyolefins can be non-toxic, non-contaminating and lighter than water. In one embodiment the polyolefin is polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low-density polyethylene (VLDPE), polypropylene (PP), polymethylpentene (PMP), polybutene-1 (PB-1), polyisobutylene (PIB), or any combination thereof. In one embodiment the polyolefin is polyethylene, polypropylene or a combination thereof.

Polyethylene (PE) (formula (C₂H₄)ₙ) consists of long chain polymers of ethylene and it can be produced as high-density (HDPE), medium-density (MDPE), or low-density polyethylene (LDPE). PE can be chemically synthesized by polymerization of ethane and it is highly variable since side chains can be obtained depending on the manufacturing process. LDPE has more branching than HDPE (i.e. has a high degree of short- and long-chain branching), and therefore it's intermolecular forces are weaker, its tensile strength is lower, and its resilience is higher. Also, because its molecules are less tightly packed and less crystalline due to the side branches, its density is lower. In one embodiment LDPE is defined by a density range of about 910-930 kg/m³, MDPE is defined by a density range of about 926 to 0.940 kg/m³, and/or the density range of HDPE is about 930 to 970 kg/m³.

Cross-linked polyethylene (PEX or XLPE) is a form of polyethylene with cross-linked bonds in the polymer structure, changing the thermoplastic to a thermoset. Indeed, crosslinking enhances the temperature properties of the base polymer and furthermore e.g. tensile strength, scratch resistance, and resistance to brittle fracture.

Ultra-high molecular weight polyethylene (UHMWPE) is a thermoplastic, and it is made up of extremely long chains of PE, which all align in the same direction. The extremely long chain can usually have a molecular mass between 3.5 and 7.5 million amu.

Linear low-density polyethylene (LLDPE) is a substantially linear PE with significant numbers of short branches. LLDPE differs structurally from conventional LDPE because of the absence of long chain branching.

Very low-density polyethylene (VLDPE) is a type of LLDPE with higher levels of short-chain branches than standard LLDPE. VLDPE can be defined e.g. by a density range of 0.880-0.910 g/cm³.

Polypropylene (PP) (formula (C₃H₆)ₙ) is a thermoplastic, which can be produced e.g. via chain-growth polymerization from the monomer propylene. PP is partially crystalline and non-polar. Its properties are very similar to PE, but it is e.g. slightly harder and more heat resistant.

Polymethylpentene (PMP) (i.e. poly(4-methyl-1-pentene), formula (C₆H₁₂)ₙ) is a thermoplastic polymer of 4-methyl-1-pentene. It is a high-molecular weight hydrocarbon and an extremely low density olefinic commodity thermoplastic. PMP's chemical resistance is close to that of PP. Compared to PP it is more easily softened by unsaturated and aromatic hydrocarbons, and chlorinated solvents, and slightly more susceptible to attack by oxidizing agents.

Polybutene-1 (PB-1) (formula (C₄H₈)ₙ) is a high molecular weight, linear, isotactic, and semi-crystalline polymer. Polybutylene can be produced by polymerization of 1-butene using supported Ziegler-Netta catalysts.

Polyisobutylene (PIB) (formula (C₄H₈)ₙ) can be prepared by polymerization of isobutene. The molecular weight of the PIB can determine the application. For example, low MW PIBs can be used as plasticizers, and medium and high MW PIBs in adhesives.

In one embodiment of the invention the enzyme capable of degrading a hydrocarbon chain, a hydrocarbon chain containing material, a polyolefin or a polyolefin containing material is from a bacterium (gram-positive or gram-negative) or fungus, and/or the micro-organism capable of degrading a hydrocarbon chain, a polyolefin or a polyolefin containing material is a bacterium (gram-positive or gram-negative) or fungus. As used herein “fungus”, “fungi” and “fungal” refer to yeast and filamentous fungi (i.e. moulds). In one embodiment of the invention the fungus is a yeast or filamentous fungus.

In one embodiment a long hydrocarbon chain or a long hydrocarbon chain like structure has a chain length of at least C10, C12, C14, C16, C18, C20, C22, C24, C26, C28, C30, C32, C34, C36, C38, C40, C45, C50, C60, C70, C80, C90 or C100. In one embodiment, the length of the hydrocarbon chain degraded or degradable by the present invention is at least C10, C12, C14, C16, C18, C20, C22, C24, C26, C28, C30, C32, C34, C36, C38, C40, C45, C50, C60, C70, C80, C90, C100, C150, C200, C250, C300, C350, C400, C450 or C500.

As used herein, “degradation” of a hydrocarbon chain, a polyolefin, plastic, synthetic or non-synthetic polymer refers to either partial or complete degradation of a hydrocarbon chain, plastic, synthetic or non-synthetic polymer to a shorter hydrocarbon chain (such as a hydrocarbon chain comprising one or more organic compounds, a long ketone, a long alcohol, a long fatty acid), oligomers and/or monomers. Said degradation can also include lowering of the molecular weight of a hydrocarbon chain or polymer, lowering of the average molecular weight, lowering of the molar mass in the peak of maximum and/or increase in polydispersity of a hydrocarbon chain or polymer. Indeed, any loss in the chain length of a hydrocarbon chain or polymer can e.g., lower tensile strength. “Enzymatic or microbial degradation” refers to a degradation caused by an enzyme or micro-organism, respectively. According to some hypothesis, in the microbial degradation the larger polymers are initially degraded by secreted exoenzymes or by outer membrane bound enzymes into smaller subunits (different length oligomers) that can be incorporated into the cells of micro-organisms and further degraded through the classical degradation pathways to yield energy and/or suit as building blocks for catabolism or metabolism.

Many plastics or other materials are mixtures comprising synthetic or semi-synthetic polymers and furthermore solubilizers and optionally other chemical agents for altering the mechanical and physical properties of said plastics or materials. For example, the plastic material may contain an additive, which increases the hydrophilicity of the plastic and makes it more prone to the enzymatic degradation. The solubilizers and other chemical compounds may also be targets of enzymatic or microbial biodegradation.

In one embodiment of the invention the enzyme (or a fragment thereof), micro-organism or host cell comprises polyolefin, PE, PEX, UHMWPE, HDPE, MDPE, LLDPE, LDPE, VLDPE PP, PMP, PB-1, or PIB degrading activity, or any combination thereof; or is capable of degrading a polyethylene and/or a polypropylene. In one embodiment the enzymes, fragments, micro-organisms or host cells of the present invention can be capable of utilizing short, medium-sized and/or long hydrocarbon chain substrates (such as those having a molecular weight of 100 Da-50 000 kDa, e.g. 5 000 Da-10 000 kDa) or any polyolefins, including but not limited to short, medium-sized and/or long hydrocarbon chain polyolefins.

Degradation of a hydrocarbon chain, a polyolefin, a material comprising a polyolefin, synthetic polymer or plastic can result in at least one or more degradation products. In one embodiment of the invention, at least one or more degradation products selected from the group consisting of an alkane, alkene, alkyne, cycloalkane, alkadiene, ketone (e.g., ketone C2-C32), fatty acid, alcohol, aldehyde, epoxy, benzene, styrene, diacid, 2-decanone, 2-dodecanone, 2-tetradecanone, 2-hexadecanone, 2-heptadecanone and 2-dotriacontanone are obtained or obtainable by the degradation of the hydrocarbon chain. For example, PE can be degraded to an alkane, alkene, alkyne, cycloalkane, alkadiene, ketone (e.g., ketone C2-C32), fatty acid, alcohol, aldehyde, diacid, 2-decanone, 2-dodecanone, 2-tetradecanone, 2-hexadecanone, 2-heptadecanone and/or 2-dotriacontanone. And for example, PP can be degraded to an alkane, alkene, alkyne, cycloalkane, alkadiene, ketone (e.g., ketone C2-C32), fatty acid, alcohol, aldehyde, and/or diacid.

In one embodiment of the invention only the enzyme(s) or micro-organism(s) or a combination thereof is (are) needed for a biotechnical or enzymatic degradation of a hydrocarbon chain, a combination of different types of hydrocarbon chains, a polyolefin or a combination of different types of polyolefins. In other words, no other degradation methods such as UV light or mechanical disruption or chemical degradation are needed in said embodiment. In other embodiments, biotechnical, enzymatic or microbial degradation can be combined with one or more other degradation methods (e.g., non-enzymatic degradation methods) including but not limited to UV light, gamma irradiation, microwave treatment, mechanical disruption and/or chemical degradation. In one embodiment of the invention the method of degrading a hydrocarbon chain is a biotechnical method, or the method comprises degradation of the hydrocarbon chain by non-enzymatic methods or means. Nonenzymatic, non-microbial or non-biotechnical degradation methods or steps including pretreatments can be carried out sequentially (e.g., before or after) or simultaneously with the biotechnical, microbial, or enzymatic degradation. For example, the hydrocarbon chains can be oxidized using e.g., oxides, such as hydrogen peroxide, in order to make the hydrocarbon chains more hydrophilic and thus more prone to the enzymatic degradation. In addition, solvents can be used for separating polymer chains from each other before enzymatic degradation of a hydrocarbon chain or a polyolefin. One or more (pre) treatments with solvents enable micro-organisms, enzymes or fragments thereof to access and degrade hydrocarbon chain or polyolefin in the inner parts of the plastic material to be degraded. Suitable solvents for plastics or polyolefins include but are not limited to toluene, xylene, benzene, trichlorobenzene, trichloroethylene, and/or tetralin.

In one embodiment, the method of degrading a polyolefin comprises obtaining, recovering, removing, recycling and/or re-utilizing at least one of the degradation products.

In one embodiment the present invention concerns an isolated enzyme or a fragment thereof comprising one or more amino acids selected from the group comprising or consisting of Leu15, Pro17, His27, His31, His32, Tyr35, Asn40, His82, Trp86, Ile105, Glyl28, Ser129, Asp164, Trp166, Glu167, His168, Tyr171, and Asn176 corresponding to the amino acid positions presented in SEQ ID NO: 2, wherein said enzyme or fragment is capable of degrading a hydrocarbon chain. In one embodiment, the present invention relates to an isolated enzyme or a fragment thereof comprising one or more amino acids selected from the group comprising or consisting of His27 His31, His32, Tyr35, Asn40, Trp166, Glu167, His168 and Tyr171 corresponding to the amino acid positions presented in SEQ ID NO: 2, wherein said enzyme or fragment is capable of degrading a hydrocarbon chain.

In one embodiment, the present invention relates an isolated enzyme or a fragment thereof comprising one or more amino acids selected from the group comprising or consisting of His31, Tyr35, Glu167, His168 and Tyr171 corresponding to the amino acid positions presented in SEQ ID NO: 2, wherein said enzyme or fragment is capable of degrading a hydrocarbon chain.

In one embodiment, the present invention relates to an isolated enzyme or a fragment thereof comprising the amino acids His31, Tyr35, Glu167, His168 and Tyr171 corresponding to the amino acid positions presented in SEQ ID NO: 2, wherein said enzyme or fragment is capable of degrading a hydrocarbon chain.

In one embodiment, the isolated enzyme or fragment thereof degrading the hydrocarbon chain, comprises the amino acids His31, Tyr35, Glu167, His168 and Tyr171, and one or more amino acids selected from the group comprising or consisting of Leu15, Pro17, His27, His32, Asn40, His82, Trp86, Ile105, Glyl28, Ser129, Asp164, Trp166, and Asn176 corresponding to the amino acid positions presented in SEQ ID NO: 2.

In one embodiment, the isolated enzyme or fragment thereof degrading the hydrocarbon chain, comprises the amino acids His31, Tyr35, Glu167, His168 and Tyr171, and one or more amino acids selected from the group comprising or consisting of His27, His32, Asn40, and Trp166 corresponding to the amino acid positions presented in SEQ ID NO: 2.

In one embodiment, the present invention relates to an isolated enzyme or a fragment thereof comprising the amino acids His27 His31, His32, Tyr35, Asn40, Trp166, Glu167, His168 and Tyr171 corresponding to the amino acid positions presented in SEQ ID NO: 2, wherein said enzyme or fragment is capable of degrading a hydrocarbon chain.

In one embodiment, the isolated enzyme or fragment thereof degrading the hydrocarbon chain, comprises the amino acids His27 His31, His32, Tyr35, Asn40, Trp166, Glu167, His168, and Tyr171, and one or more amino acids selected from the group comprising or consisting of Leu15, Pro17, His82, Trp86, Ile105, Glyl28, Ser129, Asp164 and Asn176 corresponding to the amino acid positions presented in SEQ ID NO: 2.

In one embodiment the present invention concerns an isolated enzyme or a fragment thereof having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain or a polyolefin.

Also, the present invention concerns a micro-organism or a host cell comprising an enzyme or a fragment thereof comprising one or more amino acids selected from the group comprising or consisting Leu15, Pro17, His27, His31, His32, Tyr35, Asn40, His82, Trp86, Ile105, Glyl28, Ser129, Asp164, Trp166, Glu167, His168, Tyr171, and Asn176 corresponding to the amino acid positions presented in SEQ ID NO: 2, wherein said enzyme or fragment is capable of degrading a hydrocarbon chain.

In one embodiment, the present invention relates to a micro-organism or a host cell comprising an isolated enzyme or a fragment thereof comprising one or more amino acids selected from the group comprising His27 His31, His32, Tyr35, Asn40, Trp166, Glu167, His168 and Tyr171 corresponding to the amino acid positions presented in SEQ ID NO: 2, wherein said enzyme or fragment is capable of degrading a hydrocarbon chain.

In one embodiment, the present invention relates to a micro-organism or a host cell comprising an isolated enzyme or a fragment thereof comprising one or more amino acids selected from the group comprising His31, Tyr35, Glu167, His168 and Tyr171 corresponding to the amino acid positions presented in SEQ ID NO: 2, wherein said enzyme or fragment is capable of degrading a hydrocarbon chain.

In one embodiment, the present invention relates to a micro-organism or a host cell comprising an isolated enzyme or a fragment thereof comprising the amino acids His31, Tyr35, Glu167, His168 and Tyr171 corresponding to the amino acid positions presented in SEQ ID NO: 2, wherein said enzyme or fragment is capable of degrading a hydrocarbon chain.

In one embodiment, the enzyme or fragment thereof degrading the hydrocarbon chain, comprises the amino acids His31, Tyr35, Glu167, His168 and Tyr171, and one or more amino acids selected from the group comprising Leu15, Pro17, His27, His32, Asn40, His82, Trp86, Ile105, Glyl28, Ser129, Asp164, Trp166, and Asn176 corresponding to the amino acid positions presented in SEQ ID NO: 2.

In one embodiment, the enzyme or fragment thereof degrading the hydrocarbon chain, comprises the amino acids His31, Tyr35, Glu167, His168 and Tyr171, and one or more amino acids selected from the group comprising His27, His32, Asn40, and Trp166 corresponding to the amino acid positions presented in SEQ ID NO: 2.

In one embodiment, the present invention relates to a micro-organism or a host cell comprising an isolated enzyme or a fragment thereof comprising the amino acids His27 His31, His32, Tyr35, Asn40, Trp166, Glu167, His168 and Tyr171 corresponding to the amino acid positions presented in SEQ ID NO: 2, wherein said enzyme or fragment is capable of degrading a hydrocarbon chain.

In one embodiment, the present invention relates to a micro-organism or a host cell comprising an enzyme or a fragment thereof comprising the amino acids His27 His31, His32, Tyr35, Asn40, Trp166, Glu167, His168, and Tyr171, and one or more amino acids selected from the group comprising Leu15, Pro17, His82, Trp86, Ile105, Glyl28, Ser129, Asp164 and Asn176 corresponding to the amino acid positions presented in SEQ ID NO: 2.

Further, the present invention concerns a micro-organism or a host cell comprising an enzyme or a fragment thereof having at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, wherein said enzyme or fragment is capable of degrading a hydrocarbon chain or a polyolefin.

Said relevant or specific amino acids can be e.g., consensus or conserved amino acids. As used herein “a consensus amino acid” refers to an amino acid which is the one occurring most frequently at that amino acid site in the different sequences e.g. across species. As used herein “conserved amino acids” refers to identical or similar amino acids in polypeptides or proteins across species. Conservation indicates that an amino acid has been maintained by natural selection.

The enzyme of the present invention refers to not only fungal or bacterial but also any other enzyme homologue from any micro-organism, organism or mammal. Also, all isozymes, isoforms and variants are included with the scope of said enzyme. In one embodiment of the method, enzyme, fragment, micro-organism or host cell of the present invention, the enzyme originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

In one embodiment “an enzyme of a bacterium or fungus” refers to a situation, wherein the amino acid sequence of the enzyme has the same amino acid sequence as a wild type enzyme of a bacterium or fungus (e.g. any of the above listed bacteria or fungus) or the amino acid sequence of the enzyme has a high sequence identity (e.g. 50% or more, 60% or more, 70% or more, 80% or more, 85% or more, 90% or more, or 95% or more) to an amino acid sequence of a wild type bacterial or fungal enzyme (e.g. of any of the above listed bacteria or fungus). In other words, the amino acid sequence of the enzyme used in the present invention can be modified (e.g. genetically modified).

In one embodiment, the enzyme, fragment, micro-organism or host cell is a genetically modified enzyme, fragment, micro-organism or host cell. In a specific embodiment the enzyme, fragment, micro-organism or host cell has an increased ability to degrade a hydrocarbon chain or a polyolefin compared to the corresponding unmodified enzyme, fragment, micro-organism or host cell, respectively. In one embodiment the enzyme, micro-organism or host cell comprises a genetic modification increasing an enzyme activity or the amount of a specific enzyme in a micro-organism or host cell. Genetic modifications (e.g., resulting in increased enzyme activity, increased expression of an enzyme, or increased or faster degradation of a hydrocarbon chain) include but are not limited to genetic insertions, deletions, disruptions or substitutions of one or more genes or a fragment(s) thereof or insertions, deletions, disruptions or substitutions of one or more nucleotides (e.g., insertion of a polynucleotide encoding an enzyme), or addition of plasmids. For example, one or several polynucleotides encoding an enzyme of interest can be integrated to the genome of a micro-organism or host cell. As used herein “disruption” refers to insertion of one or several nucleotides into a gene or polynucleotide sequence resulting in a lack of the corresponding polypeptide or enzyme or presence of non-functional polypeptide or enzyme with lowered activity. Methods for making any genetic modifications or modifying micro-organisms or host cells (e.g. by adaptive evolution strategy) are generally well known by a person skilled in the art and are described in various practical manuals describing laboratory molecular techniques.

In one embodiment the enzyme or a fragment thereof has one or more genetic modifications (e.g. a targeted mutation or a modification by an adaptive evolution) after one or more amino acids corresponding to the amino acids selected from the group comprising or consisting of Leu15, Pro17, His27, His31, His32, Tyr35, Asn40, His82, Trp86, Ile105, Glyl28, Ser129, Asp164, Trp166, Glu167, His168, Tyr171, and Asn176 presented in SEQ ID NO: 2. As used herein “after one or more amino acids” refers to immediately after said amino acid(s) e.g. a modification at least in the next amino acid or later after said amino acid (e.g. 1-50 amino acids, 1-30 amino acids, 1-20 amino acids, 1-10 amino acids or 1-5 amino acids after the specific amino acid mentioned above in the list of this paragraph).

As used herein “increased degradation (activity/ability/capability) of a hydrocarbon chain or a polyolefin” or “faster degradation (activity/ability/capability) of a hydrocarbon chain or a polyolefin” of an enzyme or micro-organism refers to the presence of higher activity or more activity of an enzyme or micro-organism, when compared to another enzyme or micro-organism, e.g., a genetically unmodified (wild type) enzyme or micro-organism. “Increased or faster degradation” may result e.g., from the presence of a specific enzyme in a micro-organism or an up-regulated gene or polypeptide expression in a micro-organism or an increased secretion of an enzyme by a micro-organism. Also, “increased or faster degradation” may result e.g., from the presence of (enhancing) mutations of a specific enzyme having degradation capability.

As used herein “up-regulation of the gene or polypeptide expression” refers to excessive expression of a gene or polypeptide by producing more products (e.g. mRNA or polypeptide, respectively) than an unmodified micro-organism. For example, one or more copies of a gene or genes may be transformed to a cell (e.g. to be integrated to the genome of the cell) for upregulated gene expression. The term also encompasses embodiments, where a regulating region such as a promoter or promoter region has been modified or changed or a regulating region (e.g. a promoter) not naturally present in the micro-organism has been inserted to allow the over-expression of a gene. Also, epigenetic modifications such as reducing DNA methylation or histone modifications as well as classical mutagenesis are included in “genetic modifications”, which can result in an upregulated expression of a gene or polypeptide. As used herein “increased or up-regulated expression” refers to an increased expression of the gene or polypeptide of interest compared to a wild type micro-organism without the genetic modification. Expression or increased expression can be proved for example by western, northern or southern blotting or quantitative PCR or any other suitable method known to a person skilled in the art. As used herein “increased secretion of an enzyme by a micro-organism” refers to a secretion of an enzyme outside of a cell, which produces said enzyme. Increased secretion may be caused e.g. by an increased or up-regulated expression of the gene or polypeptide of interest or by improved secretion pathway of the cell or molecules participating in the secretion of said enzyme. In one embodiment secretion of an enzyme can be increased by adding one or more glycosylation sites to the enzyme or by altering or deleting one or more glycosylation sites.

In one embodiment the genetically modified enzyme, micro-organism, host cell or polynucleotide is a recombinant enzyme, micro-organism, host cell or polynucleotide. As used herein, “a recombinant enzyme, micro-organism, host cell or polynucleotide” refers to any enzyme, micro-organism, host cell or polynucleotide that has been genetically modified to contain different genetic material compared to the enzyme, micro-organism, host cell or polynucleotide before modification (e.g. comprise a deletion, substitution, disruption or insertion of one or more nucleic acids or amino acids e.g. including an entire gene(s) or parts thereof). The recombinant micro-organism or host cell may also contain other genetic modifications than those specifically mentioned or described in the present disclosure. Indeed, the micro-organism or host cell may be genetically modified to produce, not to produce, increase production or decrease production of e.g., other polynucleotides, polypeptides, enzymes, or compounds than those specifically mentioned in the present disclosure. In certain embodiments, the genetically modified micro-organism or host cell includes a heterologous polynucleotide or enzyme. The micro-organism or host cell can be genetically modified by transforming it with a heterologous polynucleotide sequence that encodes a heterologous polypeptide. For example, a cell may be transformed with a heterologous polynucleotide encoding an enzyme of the present invention either without a signal sequence or with a signal sequence. Alternatively, for example heterologous promoters or other regulating sequences can be utilized in the micro-organisms, host cells or polynucleotides of the invention. As used herein “a heterologous polynucleotide or enzyme” refers to a polynucleotide or enzyme, which does not naturally occur in a cell or micro-organism. In one embodiment of the present invention, the enzyme or fragment thereof is encoded by a heterologous polynucleotide sequence and optionally expressed by a micro-organism or host cell.

Genetic modifications may be carried out using conventional molecular biological methods. Genetic modification (e.g. of an enzyme or micro-organism) can be accomplished in one or more steps via the design and construction of appropriate vectors and transformation of the micro-organism cell with those vectors. For example, electroporation, protoplast-PEG and/or chemical (such as calcium chloride or lithium acetate based) transformation methods can be used. Also, any commercial transformation methods are appropriate. Suitable transformation methods are well known to a person skilled in the art.

The term “vector” refers to a nucleic acid compound and/or composition that transduces, transforms, or infects a micro-organism or a host cell, thereby causing the cell to express polynucleotides and/or proteins other than those native to the cell, or in a manner not native to the cell. An “expression vector” contains a sequence of nucleic acids to be expressed by the modified micro-organism. Optionally, the expression vector also comprises materials to aid in achieving entry of the nucleic acids into the micro-organism, such as a virus, liposome, protein coating, or the like. The expression vectors contemplated for use in the present invention include those into which a nucleic acid sequence (i.e. polynucleotide) can be inserted, along with any preferred or required operational elements. Further, the expression vector must be one that can be transferred into a micro-organism or host cell and replicated therein. Vectors can be circularized or linearized and may contain restriction sites of various types for linearization or fragmentation. In specific embodiments expression vectors are plasmids, particularly those with restriction sites that have been well documented and that contain the operational elements preferred or required for transcription of the nucleic acid sequence. Such plasmids, as well as other expression vectors, are well known to those of ordinary skill in the art. Useful vectors may for example be conveniently obtained from commercially available micro-organism, yeast or bacterial vectors. Successful transformants can be selected using the attributes contributed by the marker or selection gene. Screening can be performed e.g., by PCR or Southern analysis to confirm that the desired genetic modifications (e.g., deletions, substitutions or insertions) have taken place, to confirm copy number or to identify the point of integration of nucleic acids (i.e. polynucleotides) or genes into the micro-organism cell's genome.

Indeed, the present invention also relates to a polynucleotide encoding the enzyme of the present invention or a fragment thereof, and an expression vector or plasmid comprising said polynucleotide of the present invention.

In a specific embodiment the enzyme of the present invention comprises or has a sequence having at least 20%, 25%, 30%, 35%, 40%, 45%, 50%, 55%, 60%, 65%, 70%, 71%, 72%, 73%, 74%, 75%, 76%, 77%, 78%, 79%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99% (e.g. 99.1%, 99.2%, 99.3%, 99.4%, 99.5%, 99.6%, 99.7%, 99.8% or 99.9%) or 100% sequence identity to SEQ ID NO: 2, 4, 6, 8, or 10, or an enzymatically active fragment or variant thereof. Said enzyme can be genetically modified (i.e., differs from the wild type enzyme) or unmodified. In a specific embodiment an enzyme is an isolated enzyme.

In one embodiment of the invention the enzyme has at least 20, 25, 26, 27, 28, 29, 30, 35, 40, 45, 50, 55, 60, 65, 70, 71, 72, 73, 74, 75, 76, 77, 78, 79, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99 (e.g. 99.1, 99.2, 99.3, 99.4, 99.5, 99.6, 99.7, 99.8 or 99.9%), or 100% sequence identity to SEQ ID NO: 2 (SEQ ID NO: 2 is a Bacillus licheniformis superoxide dismutase amino acid sequence).

In one embodiment, the enzyme or fragment comprises a signal sequence, e.g., a heterologous signal sequence or a signal sequence of an exogenous host cell producing said enzyme of a fragment thereof. The signal sequence can be located e.g. after or before the amino acid sequence of the enzyme e.g. for secreting said enzyme outside of the cell. The signal sequence can be any signal sequence i.e. a short polypeptide present at the N-terminus of synthesized polypeptides that are destined towards the secretory pathway, said polypeptides including but not limited to those polypeptides that are targeted inside specific organelles, secreted from the cell, or inserted into cellular membranes. In one embodiment the enzyme or fragment thereof comprises a signal sequence, does not comprise a detectable signal sequence, is secreted out of the cell which produces it, and/or is not secreted out of the cell which produces it. In one embodiment the enzyme or fragment thereof does not comprise a detectable signal sequence and is secreted out of the cell which produces it.

A polynucleotide of the present invention encodes the enzyme of the present invention or a fragment thereof. In a specific embodiment the polynucleotide comprises a sequence having a sequence identity of at least 15%, 20%, 25%, 30%, 35%, 40%, 45%, 50%, 55%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% to SEQ ID NO: 1, 3, 5, 7, 9 or 11, or a variant thereof. Said polynucleotide can be genetically modified (i.e. differs from the wild type polynucleotide) or unmodified. In a specific embodiment the polynucleotide is an isolated polynucleotide.

Identity of any sequence or fragments thereof compared to the sequence of this disclosure refers to the identity of any sequence compared to the entire sequence of the present invention. As used herein, the % identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of identity percentage between two sequences can be accomplished using mathematical algorithms available in the art. This applies to both amino acid and nucleic acid sequences. As an example, sequence identity may be determined by using BLAST (Basic Local Alignment Search Tools) or FASTA (FAST-AII). In the searches, setting parameters “gap penalties” and “matrix” are typically selected as default. In one embodiment the sequence identity is determined against the full length sequence of the present disclosure.

Nucleic acid and amino acid databases (e.g., GenBank) can be used for identifying a polypeptide having an enzymatic activity or a polynucleotide sequence encoding said polypeptide. Sequence alignment software such as BLASTP (polypeptide), BLASTN (nucleotide) or FASTA can be used to compare various sequences. Briefly, any amino acid sequence having some homology to a polypeptide having enzymatic activity, or any nucleic acid sequence having some homology to a sequence encoding a polypeptide having enzymatic activity can be used as a query to search e.g. GenBank. Percent identity of sequences can conveniently be computed using BLAST software with default parameters. Sequences having an identities score and a positive score of a given percentage, using the BLAST algorithm with default parameters, are considered to be that percent identical or homologous.

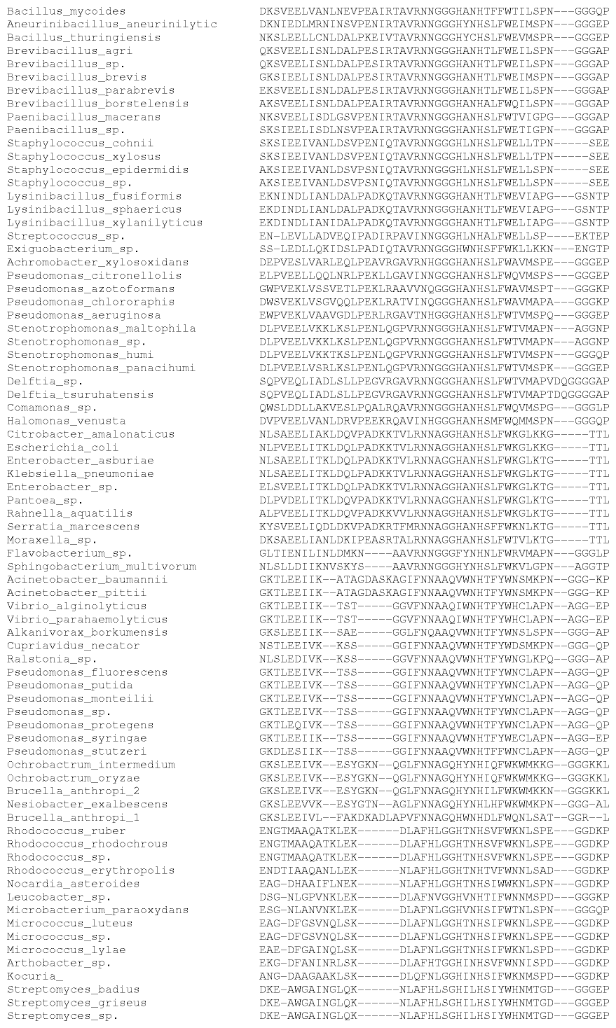

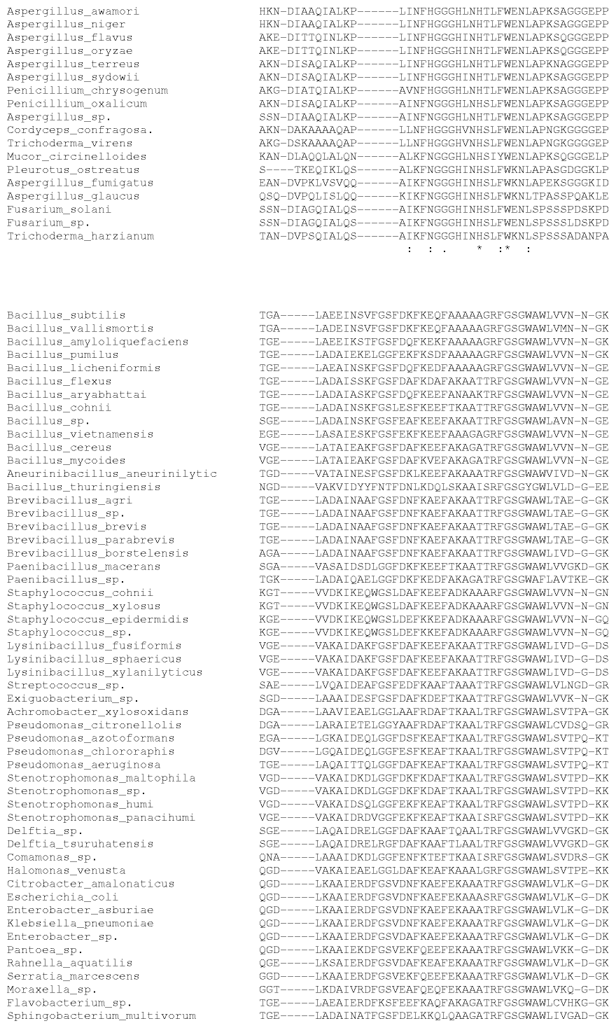

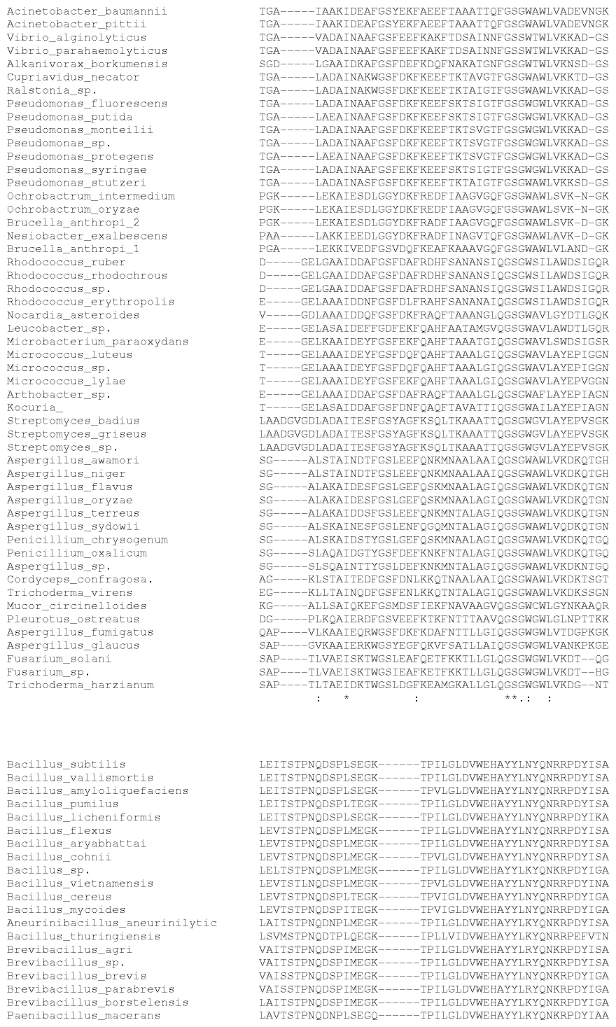

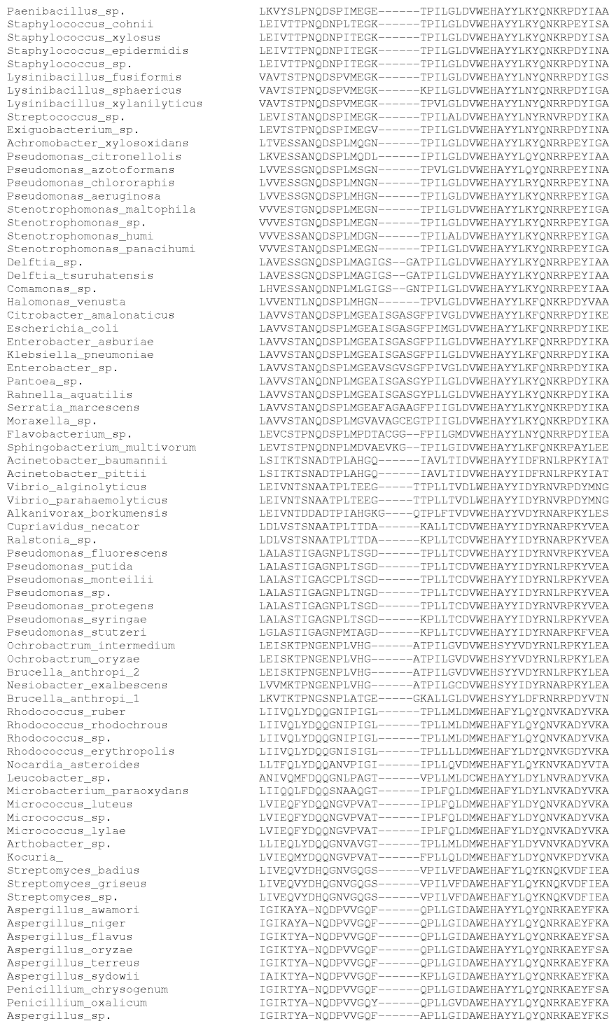

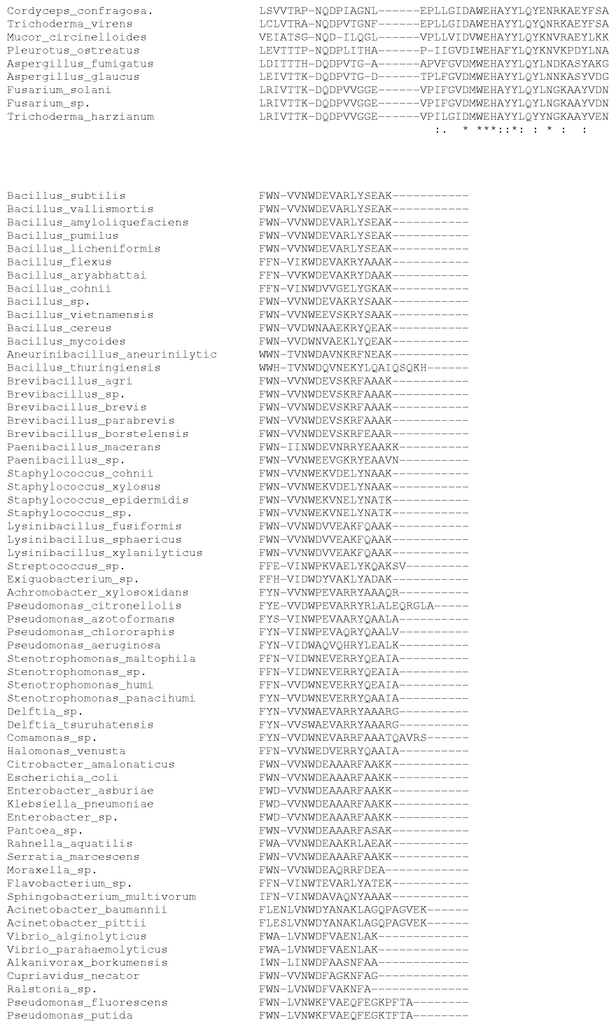

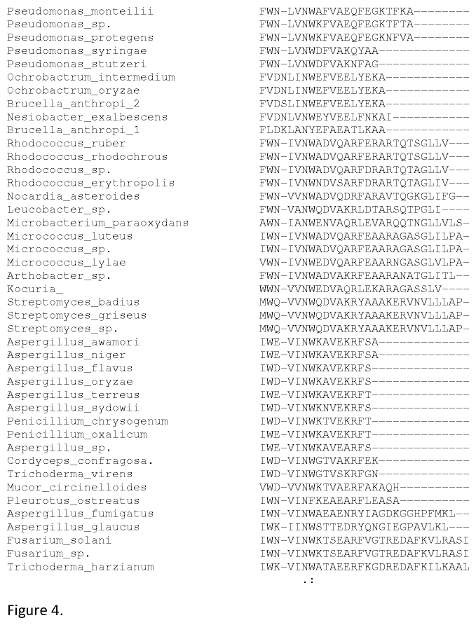

For example, an enzyme comprising a hydrocarbon chain or a polyolefin degrading activity and e.g. comprising amino acids Leu15, Pro17, His27, His31, His32, Tyr35, Asn40, His82, Trp86, Ile105, Glyl28, Ser129, Asp164, Trp166, Glu167, His168, Tyr171, and Asn176 corresponding to the amino acid positions presented in SEQ ID NO: 2, can be found as described in example 4. First, sequences containing similar kind of motifs can be searched e.g. with HMMER. HMMER is used for searching sequence databases for sequence homologs, and for making sequence alignments. It implements methods using probabilistic models called profile hidden Markov models (profile HMMs) (Robert D. Finn, Jody Clements, Sean R. Eddy (2011) HMMER web server: interactive sequence similarity searching. Nucleic Acids Research, Volume 39, Issue suppl_2, 1 Jul. 2011, Pages W29-W37, https://doi.org/10.1093/nar/gkr367). With the detected amino acid sequences or part of them or amino acid sequence(s) of previously known enzyme(s) sequence similarity searches against SEQ ID NO: 2 can be carried out e.g. by sequence alignment with ClustalW programme (https://www.genome.jp/toolsbin/clustalw) to detect corresponding consensus amino acids and their positions in amino acid sequence of interest. (See e.g. FIG. 4.)

In one embodiment one or more of the amino acids Leu15, Pro17, His27, His31, His32, Tyr35, Asn40, His82, Trp86, Ile105, Glyl28, Ser129, Asp164, Trp166, Glu167, His168, Tyr171, and Asn176 (corresponding to the amino acid positions presented in SEQ ID NO: 2) are critical for the activity of the enzyme, e.g. degradation of a substrate. In one embodiment one or more of the amino acids His27 His31, His32, Tyr35, Asn40, Trp166, Glu167, His168 and Tyr171 (corresponding to the amino acid positions presented in SEQ ID NO: 2) are critical for the activity of the enzyme, e.g. degradation of a substrate. The enzyme can comprise one or more specific amino acids or amino acid motifs for example affecting a hydrocarbon chain degrading activity (e.g. enabling different substrates and/or binding of metal ions). In one embodiment of the method, enzyme, fragment, micro-organism or host cell of the present invention, the enzyme or fragment comprises one or several amino acids selected from the group comprising Leu15, Pro17, His27, His31, His32, Tyr35, Asn40, His82, Trp86, Ile105, Glyl28, Ser129, Asp164, Trp166, Glu167, His168, Tyr171, and Asn176, wherein the amino acids and positions correspond to the amino acids and positions presented in SEQ ID NO: 2. This means that the enzyme or fragment comprises one or several amino acids, which correspond to the amino acids Leu15, Pro17, His27, His31, His32, Tyr35, Asn40, His82, Trp86, Ile105, Glyl28, Ser129, Asp164, Trp166, Glu167, His168, Tyr171, and/or Asn176 as shown in SEQ ID NO: 2. In one embodiment the enzyme or fragment comprises one, several or all amino acids Leu15, Pro17, His27, His31, His32, Tyr35, Asn40, His82, Trp86, Ile105, Glyl28, Ser129, Asp164, Trp166, Glu167, His168, Tyr171, and Asn176, wherein the amino acids and positions correspond to the amino acids and positions presented in SEQ ID NO: 2.

In one embodiment one or more of the consensus amino acids affect the degrading activity (e.g. by increasing the degrading activity) of hydrocarbon chains (e.g. Tyr35 and/or Glu167), or affect binding of a metal ion (e.g. His168).

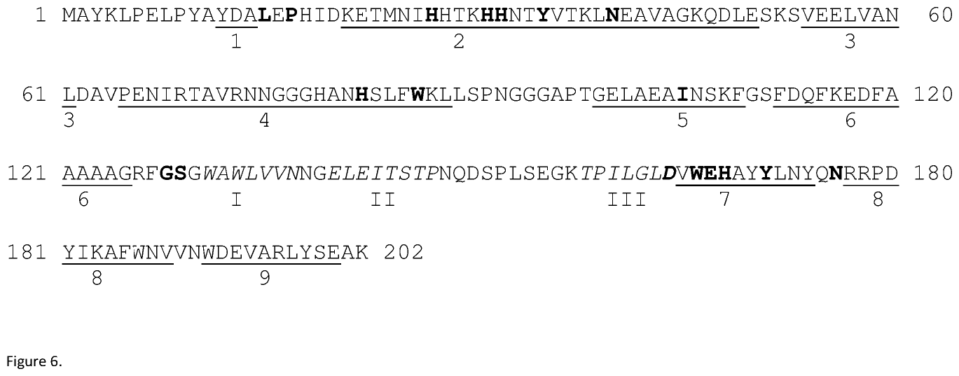

3D structure of the enzyme and positions of beta sheets and alfa helixes (2D structure) can be predicted e.g. with Phyre2 protein homology/analogy recognition engine V 2.0 (www.sbg.bio.ic.ac.uk/phyre2/html/page.cgi?id=index). The 3D and 2D structures of proteins showing the alfa helixes and beta sheets can used in predicting and finding the amino acids important for the activity of the protein. The position of critical amino acids can be localised from the predicted 2D and 3D structures as described in Example 5 and shown in FIG. 6. As can be seen from FIG. 6, of the consensus amino acids Leu15, Pro17, His27, His31, His32, Tyr35, Asn40, His82, Trp86, Ile105, Glyl28, Ser129, Asp164, Trp166, Glu167, His168, Tyr171, and Asn176 (corresponding to the amino acid positions presented in SEQ ID NO: 2), amino acids His27, His31, His32, Tyr35 and Asn40 are located in alfa helix 2, amino acids His82 and Trp86 in alfa helix 4, amino acid Ile105 in alfa helix 5, amino acids Trp166, Glu167, His168 and Tyr171 in alfa helix 7. Amino acids Leu15, Pro17, Glyl28, Ser129, Asp164 and Asn176 are located outside the alfa helixes.

In one embodiment of the method, enzyme, fragment, micro-organism or host cell of the present invention the enzyme is selected from the group comprising or consisting of superoxide dismutases. As used herein “superoxide dismutase” (SOD, EC 1.15.1.1) refers to an enzyme that reduces the amount of oxygen radicals by generating hydrogen peroxide H₂O₂ and oxygen O₂ from superoxide O₂—.

In one embodiment, the enzyme is capable of binding a divalent metal ion. In one embodiment the divalent metal ion is Zn2+, Cu2+, Ni2+, Mn2+, Fe2+, Mg2+, or any combination thereof. For example, the enzyme can bind at least Cu2+, Fe2+ or Mn2+; or Zn2+ and Cu2+. In one embodiment of the invention a divalent metal ion is part of the structure of the enzyme. In that case the enzyme cannot bind a divalent metal ion added to the culture.

In one embodiment the enzyme, fragment, micro-organism or host cell of the present invention produces hydrogen peroxide. In one embodiment another enzyme, fragment, micro-organism or host cell, optionally capable of degrading a hydrocarbon chain, uses the produced hydrogen peroxide. In one embodiment, such another enzyme is a hydrocarbon chain, such as polyolefin, degrading enzyme. In one embodiment, such another enzyme is an unspecified peroxygenase (UPO). In one embodiment, such another enzyme is a chloroperoxidase.

In one embodiment the enzyme and/or micro-organism have been genetically modified and optionally have an increased ability to degrade a hydrocarbon chain or a polyolefin compared to the corresponding unmodified enzyme and/or micro-organism, respectively.

The presence, absence or amount of specific enzyme activities can be detected by any suitable method known in the art. Specific examples of studying enzyme activities of interest are well known to a person skilled in the art. Non-limiting examples of suitable detection methods include commercial kits on market, enzymatic assays, immunological detection methods (e.g., antibodies specific for said proteins), PCR based assays (e.g., qPCR, RT-PCR), and any combination thereof.

In one embodiment, the enzymes of the present invention have high turnover rates when degrading one or more hydrocarbon chains or polyolefins, e.g. when compared to prior art enzymes. In specific embodiments the activity of an enzyme to degrade a hydrocarbon chain or a polyolefin is determined by an enzyme assay wherein said enzyme is allowed to contact with hydrocarbon chains or polyolefins (e.g. as described in example 2 and 6). In some embodiments the activity of an enzyme to degrade hydrocarbon chains or polyolefins can be determined e.g. by detecting or measuring the degradation products of hydrocarbon chains polyolefins (e.g. as shown in example 3) alternatively or by analyzing the remaining starting material containing hydrocarbon chains or polyolefins after contacting the starting material with the enzymes.

Degradation of hydrocarbon chains or polyolefins can be measured by any suitable method known in the field. In one embodiment hydrocarbon chains, polyolefins or a material comprising hydrocarbon chains or polyolefins are weighed before and/or after said hydrocarbon chains, polyolefins or material have been contacted with an enzyme, micro-organism or host cell (or any combination thereof). The presence, absence or level of degradation products of a hydrocarbon chain or polyolefin, e.g. degraded by an enzyme, micro-organism or host cell, can be detected or measured by any suitable method known in the art. Non-limiting examples of suitable detection and/or measuring methods include liquid chromatography, gas chromatography, mass spectrometry or any combination thereof (e.g. ESI-MS/MS, MaldiTof, RP-HPLC, GC-MS or LC-TOF-MS) of samples, optionally after cultivating a micro-organism or host cell e.g. 1-11 hours, 11-100 hours, or 100 hours-12 months (e.g. one, two, three, four, five, six, seven, eight, nine, ten or 11 months) or even longer in the presence of hydrocarbon chains or polyolefins (such as plastics or synthetic polymers) or after allowing a micro-organism, polypeptide or enzyme to contact with hydrocarbon chains or polyolefins. Other examples of suitable detection and/or measuring methods (including methods of fractionating, isolating or purifying degradation products) include but are not limited to filtration, solvent extraction, centrifugation, affinity chromatography, ion exchange chromatography, electrophoresis, hydrophobic interaction chromatography, gel filtration chromatography, reverse phase chromatography, chromatofocusing, differential solubilization, preparative disc-gel electrophoresis, isoelectric focusing, HPLC, gel permeation chromatography (GPC), fourier-transform infrared spectroscopy (FT-IR), NMR and/or reversed-phase HPLC.

For degradation, hydrocarbon chains, polyolefins or a material comprising hydrocarbon chains or polyolefins can be contacted with an enzyme, micro-organism or host cell (or any combination thereof) at a ratio, concentration and/or temperature for a time sufficient for the degradation of interest. Suitable time for allowing the enzyme, micro-organism, or host cell to degrade a hydrocarbon chain or hydrocarbon chains can be selected e.g. from the group consisting of 1, 2, 3, 4, 5, 6, 7, 8, 9, 10, 11, 12, 13, 14, 15, 16, 17, 18, 19, 20, 21, 22, 23, and 24 hours, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and 31 days, 1, 2, 3, 4, 5, 6, 7, 8, 9, 10, 11, and 12 weeks, and 1, 2, 3, 4, 5, 6, 7, 8, 9, 10, 11 and 12 months. The degradation may take place in liquid, semi-solid, moist or dry conditions. The degradation is conveniently conducted aerobically, microaerobically and/or anaerobically. If desired, specific oxygen up-take rate can be used as a process control. The degradation can be conducted continuously, batch-wise, feed batch-wise or as any combination thereof.

In one embodiment the enzyme(s), micro-organism(s) or host cell(s) can be utilized for degrading hydrocarbon chains or polyolefins e.g., at a temperature below 100° C. such as 15-95° C., 30-95° C., 15-50° C., 30-50° C. or 40-80° C. (e.g., 50° C.). In one embodiment the enzyme, fragment, micro-organism or host cell is capable of degrading a polyolefin at a temperature of at least 20° C., at least 25° C., at least 30° C., or at least 37° C. This indicates low energy need and therefore also moderate costs of the method.

In some embodiments of the invention an enzyme and/or enzymes (e.g. a combination of different enzymes) can produce material (e.g. degradation products (such as alkane) or modified material) for other enzymes or enzymes of other type(s) or micro-organisms to further degrade or modify said material (e.g. to fatty acids, PHA or diacids). On the other hand, in some embodiments of the invention a micro-organism, host cell, micro-organisms (e.g. a combination of different micro-organisms) or host cells can produce material (e.g. degradation products (such as alkane) or modified material) for micro-organisms of other type(s) or enzymes to further degrade or modify said material (e.g. to fatty acids, PHA or diacids).

In one embodiment, fatty acid derived products such as hydroxy fatty acids and/or diacids, and/or polyhydroxyalkanoate (PHA) can be produced from the degradation products of hydrocarbon chains, such as degradation products of polyolefins, produced by the enzymes, micro-organisms and/or host cells of the present invention using other enzymes or micro-organisms. The enzymes, micro-organisms and/or host cells of the present invention degrade the hydrocarbon chain to compounds which are used as substrate(s) by an enzyme which is able to produce fatty acid derived products such as hydroxy fatty acids, diacids, and/or polyhydroxyalkanoate (PHA). In one embodiment, PHA can be produced from the degradation products of polyethylene produced by the enzymes, micro-organisms and/or host cells of the present invention using an enzyme able to produce PHA, such as PHA synthase, or micro-organisms able to produce PHA. Examples 7 and 8 show specific embodiments of the production of hydroxy fatty acids and polyhydroxyalkanoate (PHA). In one embodiment, the micro-organism and/or host cell producing hydroxy fatty acids and/or polyhydroxyalkanoate (PHA) is selected from the micro-organisms and/or host cells of the present invention. In one embodiment, the host cell producing polyhydroxyalkanoate (PHAs) is modified to overexpress an enzyme producing PHA, such as PHA synthetase. In one embodiment, the host cell modified to overexpress an enzyme producing PHA, such as PHA synthetase, is selected from the host cells of the present invention.

As used herein “polyhydroxyalkanoates (PHAs)” are polyesters which comprise hydroxyacyls, such as 2-hydroxyacyls or 3-hydroxyacyls, having carbon chain length of at least C4, C6, C10 or C12, for example. The chemical composition of a PHA can be homo- or co-polyester. In one embodiment the PHA comprises 2-hydroxyacyls having carbon chain length of C10 and/or C12. In one embodiment the PHA comprises 3-hydroxyacyls having carbon chain length of C10 and/or C12. In one embodiment, the PHA is a medium chain length PHA.

In some embodiments of the present invention the micro-organisms or host cells are cultured under conditions (e.g., suitable conditions) in which the cultured micro-organism or host cell produces polypeptides, enzymes or compounds or interest (e.g. enzymes for degrading hydrocarbon chains or polyolefins). The micro-organisms or host cells can be cultivated in a medium containing appropriate carbon sources together with other optional ingredients selected from the group consisting of nitrogen or a source of nitrogen (such as amino acids, proteins, inorganic nitrogen sources such as nitrate, ammonia, urea or ammonium salts), yeast extract, peptone, minerals and vitamins, such as KH2PO4, Na2HPO, MgSO, CaCl2, FeCls, ZnSO, citric acid, MnSO, COCl2, CuSO, Na2MoO4, FeSO4, HsBO4, D-biotin, Ca-Pantothenate, nicotinic acid, myoinositol, thiamine, pyridoxine, p-amino benzoic acid. Suitable cultivation conditions, such as temperature, cell density, selection of nutrients, and the like are within the knowledge of a skilled person and can be selected to provide an economical process with the micro-organism in question. Temperatures may range from above the freezing temperature of the medium to about 50° C. or even higher, although the optimal temperature will depend somewhat on the particular micro-organism. In a specific embodiment the temperature is from about 25 to 35° C. The pH of the cultivation process may or may not be controlled to remain at a constant pH, but is usually between 3 and 9, depending on the production organism. Optimally the pH can be controlled e.g. to a constant pH of 7-8 (e.g. in the case of Escherichia coli) or to a constant pH of 5-6 (e.g. in the case of Yarrowia lipolytica). Suitable buffering agents include, for example, calcium hydroxide, calcium carbonate, sodium hydroxide, potassium hydroxide, potassium carbonate, hydrogen chloride, sodium carbonate, ammonium carbonate, ammonia, ammonium hydroxide and/or the like. In general, those buffering agents that have been used in conventional cultivation methods are also suitable here. The cultivation conditions can also include oxides, such as ZnO, MnO and/or TiO₂, which may affect positively on the degradation ability of the enzyme.

The micro-organisms or host cells can be normally separated from the culture medium after cultivation, before or after contacting with a hydrocarbon chain. The separated micro-organisms, host cells or a liquid (e.g. culture medium) comprising micro-organisms or host cells can be used for contacting hydrocarbon chains or polyolefins.

Polypeptides or enzymes can be secreted outside of the cells or they can stay in the cells. Therefore, the polypeptides or enzymes can be recovered from the cells or directly from the culture medium. In some embodiments both intracellular and extracellular polypeptides or enzymes are recovered. Prior to recovering, cells can be disrupted. Isolation and/or purification of polypeptides or enzymes can include one or more of the following: size exclusion, desalting, anion and cation exchange, based on affinity, removal of chemicals using solvents, extraction of the soluble proteinaceous material e.g., by using an alkaline medium (e.g., NaOH, Borate-based buffers or water is commonly used), isoelectric point-based or salt-based precipitation of proteins, centrifugation, and ultrafiltration. In one embodiment of the method, polypeptide or enzyme of the present invention, said polypeptide or enzyme is a purified or partly purified polypeptide or enzyme. If the polypeptide or enzyme is secreted outside of the cell it does not necessarily need to be purified.

In one embodiment, the enzyme or fragment thereof is immobilized. Immobilization can be carried out by any method known to a person skilled in the art such as immobilization by crosslinking e.g., with glutaraldehyde or by using hydrophopic carrier for the enzyme.

Hydrocarbon chain(s) or polyolefin(s) degrading enzymes can be expressed in any suitable host (cell). Examples of suitable host cells include but are not limited to cells of micro-organisms such as bacteria, yeast, fungi and filamentous fungi, as well as cells of plants and animals (such as mammals). Specific examples of host cells include but are not limited to Escherichia coli, Saccharomyces cerevisiae, Yarrowia lipolytica, Pichia pastoris, Trichoderma reesei, Aspergillus nidulans, Aspergillus niger, Bacillus licheniformis, Bacillus subtilis, and Myceliophthora thermophila.

In one embodiment of the invention the micro-organism(s) or host cell(s) is (are) a bacterium or bacteria or fungus selected from the group comprising or consisting of Bacillus, Paenibacillus, Achromobacter, Acinetobacter, Alcanivorax, Aneurini bacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis, and any combination thereof.

Also, the micro-organism or host cell of the present invention can be used in a combination with any other micro-organism (simultaneously or consecutively), e.g., micro-organisms can be a population of different micro-organisms degrading different hydrocarbon chains or micro-organisms can be a combination of a bacterium and a fungus (to be used simultaneously or consecutively).

The inventors of the present disclosure have been able to isolate enzymes capable of degrading hydrocarbon chains or polyolefins from micro-organisms, and use said enzymes or micro-organisms for degrading hydrocarbon chains or polyolefins and/or producing degradation products of interest.

The present invention further relates to use of the enzyme, micro-organism, host cell, polynucleotide, expression vector or plasmid of the present invention or any combination thereof for degrading a hydrocarbon chain, a polyolefin or hydrocarbon chains or polyolefins of different types.

Also, the present invention concerns a method of producing the enzyme of the present invention, wherein a recombinant micro-organism or host cell comprising the polynucleotide encoding the enzyme or fragment thereof of the present invention expresses or is allowed to express said enzyme or fragment thereof. For example, a vector or plasmid comprising the polynucleotide of interest can be transfected to a host cell, and the host cell can be used for expressing the enzyme of the present invention. In one embodiment the polynucleotide of interest is integrated into the genome of the host cell or the polynucleotide of interest is expressed from a vector or plasmid which is not integrated into the genome of the host cell. In one embodiment said expression of the enzyme can be controlled for example through inducible elements of promoters, vectors or plasmids.

As used in the present disclosure, the terms “polypeptide” and “protein” are used interchangeably to refer to polymers of amino acids of any length. As used herein “an enzyme” refers to a protein or polypeptide which is able to accelerate or catalyze (bio) chemical reactions.

As used herein “polynucleotide” refers to any polynucleotide, such as single or double-stranded DNA (genomic DNA or cDNA or synthetic DNA) or RNA (e.g. mRNA or synthetic RNA), comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used herein “isolated” enzymes, polypeptides or polynucleotides refer to enzymes, polypeptides or polynucleotides purified to a state beyond that in which they exist in cells. Isolated polypeptides, proteins or polynucleotides include e.g. substantially purified (e.g. purified to at least 10%, 20%, 30%, 40%, 50%, 60%, 70%, 80%, 90% purity) or pure enzymes, polypeptides or polynucleotides.

It is well known that a deletion, addition or substitution of one or a few amino acids of an amino acid sequence of an enzyme does not necessarily change the catalytic properties of said enzyme. Therefore, the invention also encompasses variants and fragments of the enzymes of the present invention or given amino acid sequences having the stipulated enzyme activity. The term “variant” as used herein refers to a sequence having minor changes in the amino acid sequence as compared to a given sequence. Such a variant may occur naturally e.g. as an allelic variant within the same strain, species or genus, or it may be generated by mutagenesis or other gene modification. It may comprise amino acid substitutions, deletions or insertions, but it still functions in substantially the same manner as the given enzymes, in particular it retains its catalytic function as an enzyme (e.g. capability to degrade a hydrocarbon chain). In one embodiment of the invention a fragment of the enzyme is an enzymatically active fragment or variant thereof.

A “fragment” of a given enzyme or polypeptide sequence means part of that sequence, e.g. a sequence that has been truncated at the N- and/or C-terminal end. It may for example be the mature part of an enzyme or polypeptide comprising a signal sequence, or it may be only an enzymatically active fragment of the mature enzyme or polypeptide.

It will be obvious to a person skilled in the art that, as the technology advances, the inventive concept can be implemented in various ways. The invention and its embodiments are not limited to the examples described below but may vary within the scope of the claims.

### EXAMPLES

### Example 1. Expression of Bacillus licheniformis, Bacillus cereus, Bacillus flexus, Bacillus subtilis and Rhodococcus ruber superoxide dismutases in Escherichia coli

The gene encoding Bacillus licheniformis superoxide dismutase (SEQ ID NO: 2) amino acid was cloned from genomic Bacillus licheniformis DNA by PCR by using oligonucleotides oPlastBug-242 (TAGAAATAATTTTGTTTAACTTTAAGAAGGAGATATATCCATGGCTTACAAACT TCCAGAATTACCTTATGCT, SEQ ID NO: 11) and oPlastBug-243 (CAAGCTGGGATTTAGGTGACACTATAGAATACTCAAGCTTTTATTTTGCTTCG CTGTAAAGGCGTGC, SEQ ID NO: 12). The resulting DNA fragment containing coding region of the gene (SEQ ID NO: 1) was cloned into Ncol and HindIII digested Escherichia coli expression vector pBAT4 with Gibson assembly resulting in plasmid pPB095-1 and expressed in E. coli strain Shuffle T7 Express (New England Biolabs).

The gene encoding Bacillus cereus superoxide dismutase (SEQ ID NO: 4) amino acid was cloned from genomic Bacillus cereus DNA by PCR by using oligonucleotides oPlastBug-238 (TAGAAATAATTTTGTTTAACTTTAAGAAGGAGATATATCCATGTCACTTAAGTG GCAATACATAAACTGGGA, SEQ ID NO: 13) and oPlastBug-239 (CAAGCTGGGATTTAGGTGACACTATAGAATACTCAAGCTTTTATTTTGCTTCT TGGTAACGTTTTTCAGCAGC, SEQ ID NO: 14). The resulting DNA fragment containing coding region of the gene (SEQ ID NO: 3) was cloned into Ncol and

HindIII digested Escherichia coli expression vector pBAT4 with Gibson assembly resulting in plasmid pPB093-3 and expressed in E. coli strain Shuffle T7 Express (New England Biolabs).

The gene encoding Bacillus flexus superoxide dismutase (SEQ ID NO: 6) amino acid was cloned from genomic Bacillus flexus DNA by PCR by using oligonucleotides oPlastBug-138 (ACAATTCCTCTAGAAATAATTTTGTTTAACTTTAAGAAGGAGATATATCCATGG CTTACGAATTACCACAATTACCTTATGCA, SEQ ID NO: 15) and oPlastBug-139 (TTGTTAGCAGCCGGATCAAGCTGGGATTTAGGTGACACTATAGAATACTCTT ATTTTGCTGCTGCGTAGCGTTTTGC, SEQ ID NO: 16). The resulting DNA fragment containing coding region of the gene (SEQ ID NO: 5) was cloned into Ncol and HindIII digested Escherichia coli expression vector pBAT4 with Gibson assembly resulting in plasmid pPB045-1 and expressed in E. coli strain Shuffle T7 Express (New England Biolabs).

The gene encoding Bacillus subtilis superoxide dismutase (SEQ ID NO: 8) amino acid was cloned from genomic Bacillus flexus DNA by PCR by using oligonucleotides oPlastBug-244 (TAGAAATAATTTTGTTTAACTTTAAGAAGGAGATATATCCATGGCTTACGAACT TCCAGAATTACCTTATGC, SEQ ID NO: 17) and oPlastBug-245 (CAAGCTGGGATTTAGGTGACACTATAGAATACTCAAGCTTTTATTTTGCTTCG CTGTATAGACGAGCCA, SEQ ID NO: 18). The resulting DNA fragment containing coding region of the gene (SEQ ID NO: 7) was cloned into Ncol and HindIII digested Escherichia coli expression vector pBAT4 with Gibson assembly resulting in plasmid pPB096-1 and expressed in E. coli strain Shuffle T7 Express (New England Biolabs).

The gene encoding Rhodococcus ruber superoxide dismutase (SEQ ID NO: 10) amino acid was cloned from genomic Rhodococcus ruber DNA by PCR by using oligonucleotides oPlastBug-136 (ACAATTCCTCTAGAAATAATTTTGTTTAACTTTAAGAAGGAGATATATCCATGG CTGAGTACACACTTCCGGACC, SEQ ID NO: 19) and oPlastBug-137 (TTGTTAGCAGCCGGATCAAGCTGGGATTTAGGTGACACTATAGAATACTCCT AGACCAGCAGACCGGAGGTCT, SEQ ID NO: 20). The resulting DNA fragment containing coding region of the gene (SEQ ID NO: 9) was cloned into Ncol and HindIII digested Escherichia coli expression vector pBAT4 with Gibson assembly resulting in plasmid pPB044-3 and expressed in E. coli strain Shuffle T7 Express (New England Biolabs).

Plasmid pPB044-3, pPB045-1, pPB093-3, pPB095-1 and pPB096-1 were expressed in E. coli Shuffle T7 Express grown at +37° C. in SB (30 g tryptone, 20 g yeast extract, 10 g MOPS (3-[N-morpholino]-propanesulfonic acid) per liter) media containing 100 μg/ml ampicillin. Protein expression was induced by the addition of 1 mM β-D-1-thiogalactopyranoside (IPTG), and induced cultures were further incubated at +30° C. for 24 hours. Cells were harvested by centrifugation (3184 g, 10 min RT), and supernatant was collected and filtered through 0.20 μm filter. Enzymes from fresh or −75C stored filtered samples were purified as follows: pH of filtered samples were adjusted to pH 6.0 with 1 M HCl. Additionally, samples were diluted with 1 M Tris-HCl pH 6.0 so that final concentration was 50 mM. 3 ml of samples was loaded in HiTrap Mono-Q column (1 ml column volume) equilibrated with 50 mM Tris-HCl, pH 6.0 followed by washing with 5 ml of 50 mM Tris-HCl pH 6.0. Enzymes were eluted with 2 ml of 0.25 M NaCl, 50 mM Tris-HCl, pH 6.0. Purifity of enzymes were detected with SDS-Page analysis. Partially purified enzymes were used directly in enzyme assays. As negative control purification steps were repeated with E. coli strain expressing empty pBAT4 plasmid.

### Example 2. Degradation of Polypropylene and Polyethylene with Bacillus licheniformis, Bacillus Cereus, Bacillus Flexus, Bacillus subtilis and Rhodococcus Ruber Enzymes

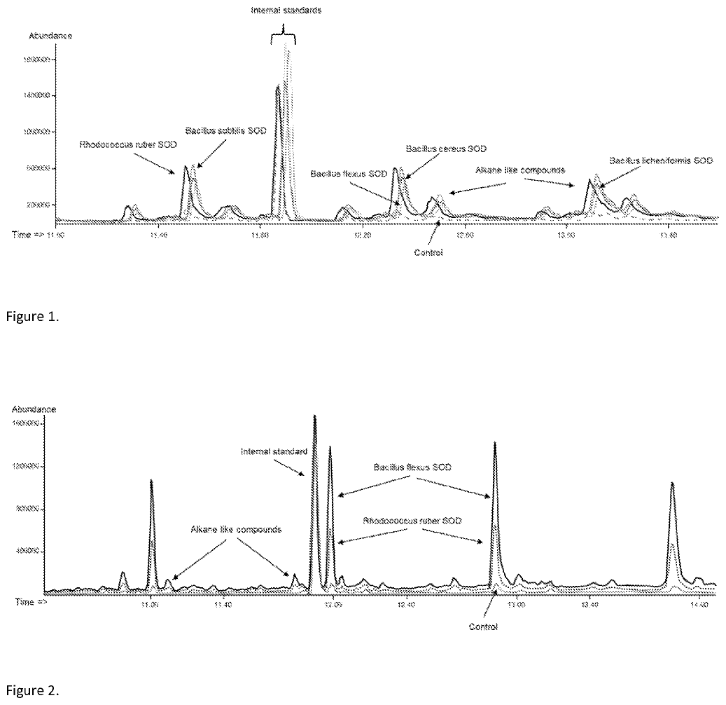

Enzyme assay with polypropylene with Bacillus licheniformis, Bacillus cereus, Bacillus flexus, Bacillus subtilis and Rhodococcus ruber enzymes were carried out with partially purified enzymes as follows: 950 μl of 50 mM McIlvaine pH 3.0 and 1 mM Mn(II)Cl2 with polypropylene powder (Licocene PP 6102 Fine grain, Clariant) was incubated with 50 μl of partially purified enzymes from Example 1 at 37° C. for 118 hours. 0.25 M NaCl, 50 mM Tris-HCl pH 6.0 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 1. With enzyme samples several peaks appeared which were missing from control sample. These peaks presented alkane like compounds or oxygen containing hydrocarbons.

Enzyme assay with polyethylene with Bacillus flexus and Rhodococcus ruber enzymes were carried out with partially purified enzymes as follows: 950 μl of 50 mM McIlvaine pH 3.0 and 1 mM Mn(II)Cl2 with with polyethylene powder (4000 Da, Sigma-Aldrich) was incubated with 50 μl of partially purified enzymes from Example 1 at 37° C. for 118 hours. 0.25 M NaCl, 50 mM Tris-HCl pH 6.0 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 2. With enzyme samples several peaks appeared which were missing from control sample. These peaks presented alkane like compounds or oxygen containing hydrocarbons.

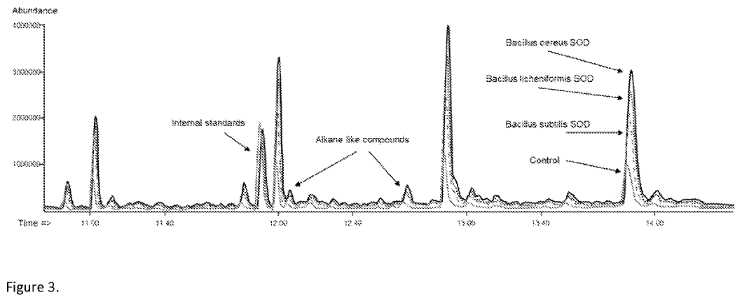

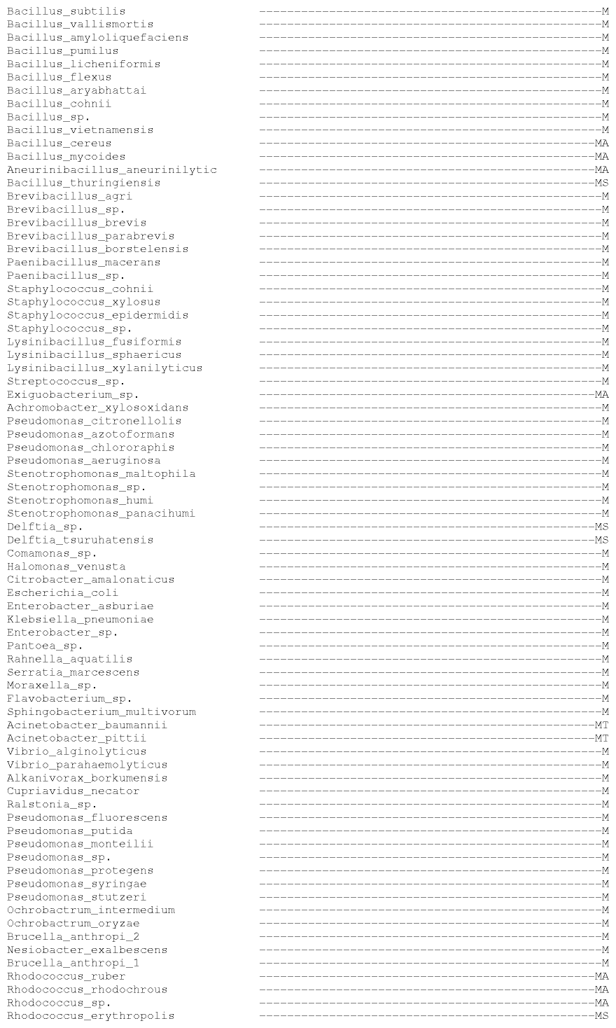

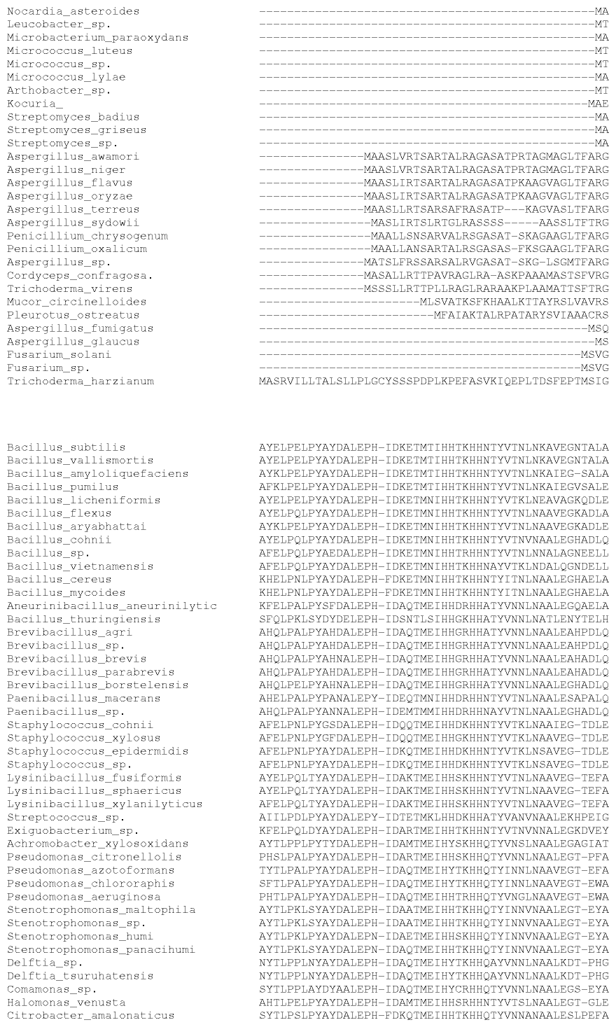

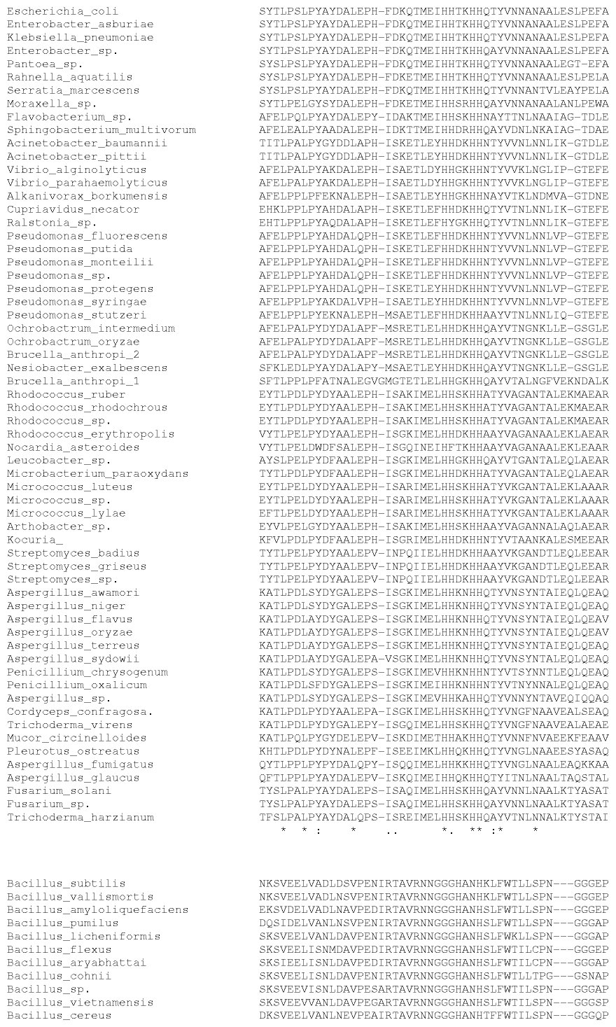

Enzyme assay with polyethylene with Bacillus licheniformis, Bacillus cereus and Bacillus subtilis enzymes were carried out with partially purified enzymes as follows: 950 μl of 50 mM HEPES pH 8.0 and 1 mM Mn(II)Cl2 with polyethylene powder (4000 Da, Sigma-Aldrich) was incubated with 50 μl of partially purified enzymes from Example 1 at 50° C. for 118 hours. 0.25 M NaCl, 50 mM Tris-HCl pH 6.0 elution sample from the purification of E. coli strain having empty plasmid (pBAT4) as described in Example 1 was used as a control. After incubation GC-MS run was carried out with liquid fraction as described in Example 3. Results from the GC-MS run are shown in FIG. 3. With enzyme samples several peaks appeared which were missing from control sample. These peaks presented alkane like compounds or oxygen containing hydrocarbons.

### Example 3. Gas Chromatography-Mass Spectrometry (GC-MS) Analysis of Volatile Degradation Products of Hydrocarbons with Superoxide Dismutase

Aliquots (300 μl) of the samples from examples 2 and 6 were transferred to new tubes and an internal standard (methyl heptadecanoate) was added. The samples were extracted with dichloromethane (200 μl) by agitating in a shaker for 15 min. After extraction the samples were allowed settle (15 minutes at room temperature), centrifuged (5 min, 10 000 rpm) and finally, the dichloromethane phase was transferred to GC-MS vials. The runs were performed on Agilent GC-MS equipped with an HP-FFAP (25 m×200 μm×0.3 μm) column and helium was used as a carrier gas. The injector temperature was 250° C., and a splitless injection mode was used. The oven temperature was 40° C. for 3 min, increased to 240° C. at 20° C./min and kept at 240° C. for 14 min. The detected mass range was 35-600 m/z and compounds were identified based on NIST08 MS library. Results from GC-MS analysis are described in Examples 2 and 6.

### Example 4. Characterisation of Amino Acid Sequence Motifs of Polyethylene and Polypropylene Degrading Superoxide Dismutases

A sequence search based on HMMER (Robert D. Finn, Jody Clements, Sean R. Eddy (2011) HMMER web server: interactive sequence similarity searching. Nucleic Acids Research, Volume 39, Issue suppl_2, 1 Jul. 2011, Pages W29-W37, https://doi.org/10.1093/nar/gkr367) was done using the amino acid sequences of SEQ ID NOs: 2, 4, 6, 8, 10 and 23. The HMMER search was carried out against the UniProtKB/TrEMBL. The results were filtered based on the e-value. Several hundreds of sequences were identified. Among these sequences amino acid sequences originating from species which have been shown to degrade polyethylene or polypropylene were collected (SEQ ID Nos: 2, 4, 6, 8, 10, 23-123) and used in multiple sequence alignment carried out with CLUSTAW (https://www.genome.jp/tools-bin/clustalw) with default parameters.

In the alignment several consensus amino acids could be detected (based on Bacillus licheniformis amino acid position): Leu15, Pro17, His27, His31, His32, Tyr35, Asn40, His82, Trp86, Ile105, Glyl28, Ser129, Asp164, Trp166, Glu167, His168, Tyr171, Asn176 (see FIG. 4).

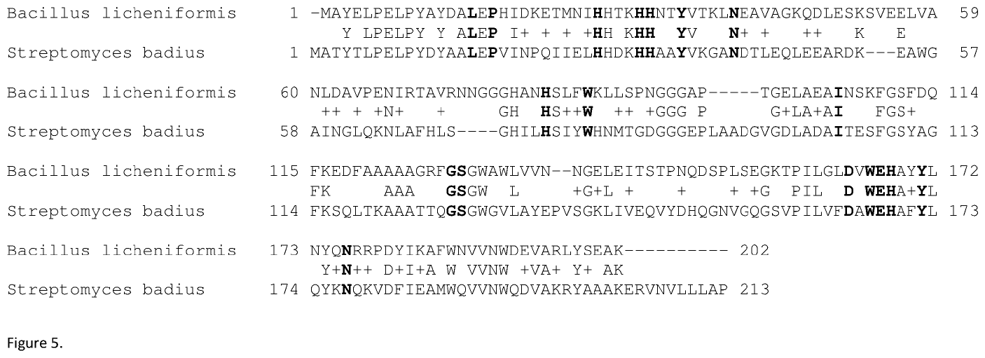

To confirm the existence and position of consensus amino acids in a specific enzyme corresponding amino acid sequence was compared to B. licheniformis superoxide dismutase (SEQ ID NO: 2) by carrying out pairwise alignment with ClustalW default parameters by using Geneious 10.2.6 programme. In FIG. 5 is an example of pairwise alignment between Streptomyces badius superoxide dismutase (SEQ ID NO: 113) and B. licheniformis superoxide dismutase (SEQ ID NO: 2). Even these amino acid sequences have only 35% identity between each other abovementioned consensus amino acids could be detected and their position in Streptomyces badius amino acid sequence identified (mark in bold).

### Example 5. Localising Consensus Amino Acids into Enzymes 2D and 3D Structure

Two-dimensional and 3 D structures of Bacillus licheniformis superoxide dismutase (SEQ ID N:O 2) was constructed with Phyre2 protein homology/analogy recognition engine V 2.0 (www.sbg.bio.ic.ac.uk/phyre2/html/page.cgi?id=index) with default parameters. The predicted alfa helixes and beta sheets were localised together with identified consensus amino acids from Example 4 into amino acid sequence shown in FIG. 6. The amino acids which in predicted 3D structure were critical to metal binding, right protein structure and activity were identified. These amino acids were in alfa helixes 2 and 7 located His27, His31, His32, Tyr35, Asn40, Trp166, Glu167, His168 and Tyr171.

### Example 6. Enzyme Activity with Purified Bacillus licheniformis Superoxide Dismutase

The enzyme was purified using ion exchange (IEX) chromatography. E. coli expressing Bacillus licheniformis superoxide dismutase was cultivated as described in Example 1. Filtered sample pH was adjusted to pH 6.0 with 1 M HCl prior stored at −75° C. The buffer of the melted culture was changed to 50 mM Tris-HCl pH 6 using PD-10 desalting columns (Cytiva) and the sample was applied on an anion exchange HiTrap Q Sepharose fast flow column (Cytiva) pre-equilibrated with 50 mM Tris-HCl pH 6. The bound proteins were eluted with a 0-250 mM linear NaCl gradient for 20-30 column volumes (CV), where after the NaCl concentration was kept at 250 mM for 2 CV followed by a linear 250-1000 mM NaCl for 5 CV. Fractions containing the enzyme, as judged by SOD enzyme assay (Sigma-Aldrich), were pooled, and concentrated using a Vivaspin sample concentrator (MWCO 5000; Sartorius, Germany). The purified enzyme was stored at −75° C.

The quality of purified protein was assessed by SDS-PAGE, to verify high enough (>85%) homogeneity of protein samples for enzyme assays. Protein concentration was determined by Bio-Rad Bradford protein assay with BSA as standard by using the standard microplate assay. Samples were made in triplicate and were incubated for 15 min and A595 was measured with Varioskan Flash (Thermo Fischer).

Enzyme assay with octadecane and octadecanoic acid with purified Bacillus licheniformis, enzyme were carried out as follows: 950 μl of 50 mM HEPES pH 8.0 and 1 mM Mn(II)Cl2 with 20 mg of octadecane or octadecanoic acid was incubated with 50 μg of purified enzyme at 50° C. for 165 hours. After incubation samples were analysed with GCMS as described in Example 3. As negative controls, enzyme assays without enzymes were carried out.

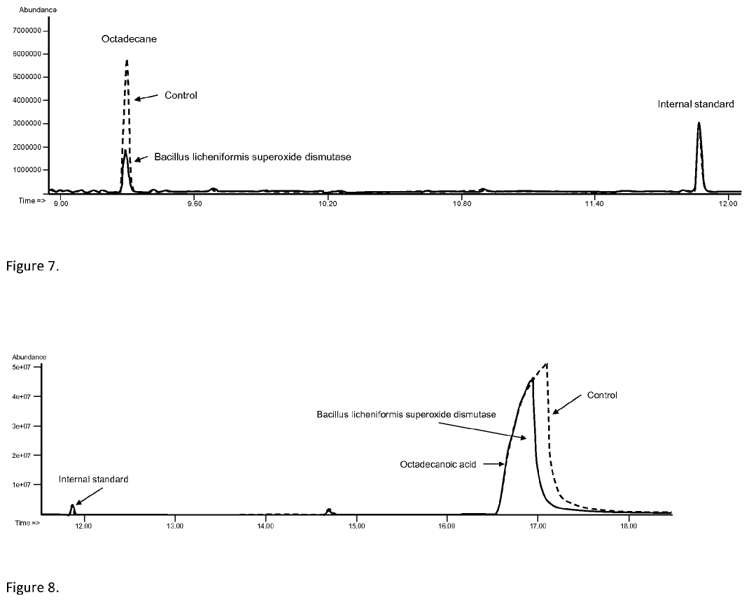

With enzyme sample clear reduction of octadecane amount could be seen compared to the control indicating degradation of octadecane (FIG. 7).

With enzyme sample clear reduction of octadecanoic acid amount could be seen compared to the control indicating degradation of octadecanoic acid (FIG. 8).

### Example 7. Expression of Bacillus flexus Superoxide Dismutase and Pseudomonas sp. PHA Synthase in Yarrowia lipolytica

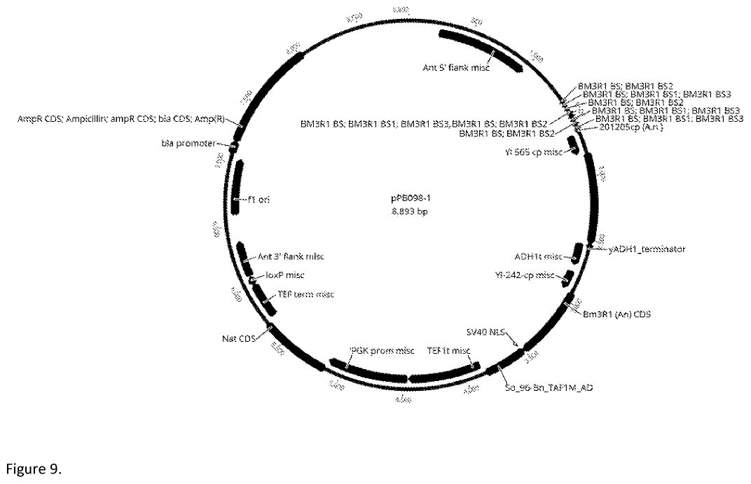

The gene encoding Bacillus flexus superoxide dismutase (SEQ ID NO: 6) amino acid with Ser83Asn and Ser115Lys mutations and with Yarrowia lipolytica LIP2 signal peptide (SEQ ID NO:21) was commercially (Genscript) synthetized with codon optimization for expression in Yarrowia lipolytica cells (SEQ ID NO: 22). Pacl and Bg/II restriction sites were included at 5′ and 3′ ends of construct for restriction digestion cloning. The constructs were cloned into Yarrowia lipolytica integration cassette plasmid B11157 digested with Pacl and Bc/I. B11157 plasmid contains flanks to ANT1 gene and SES promoter (SES promoter described in Rantasalo et al 2018. Nucleic Acids Research, Volume 46, Issue 18, 12 Oct. 2018, Page e111, https://doi.org/10.1093/nar/gky598). The resulting plasmid was named as pPB098-1 (FIG. 9). Notl digested integration fragment was transformed into VTT-C-00365 Yarrowia lipolytica strain (VTTCC) with Frozen-EZ yeast transformation kit. The transformant having B. flexus superoxide dismutase integrated was used as a host in the following modification.

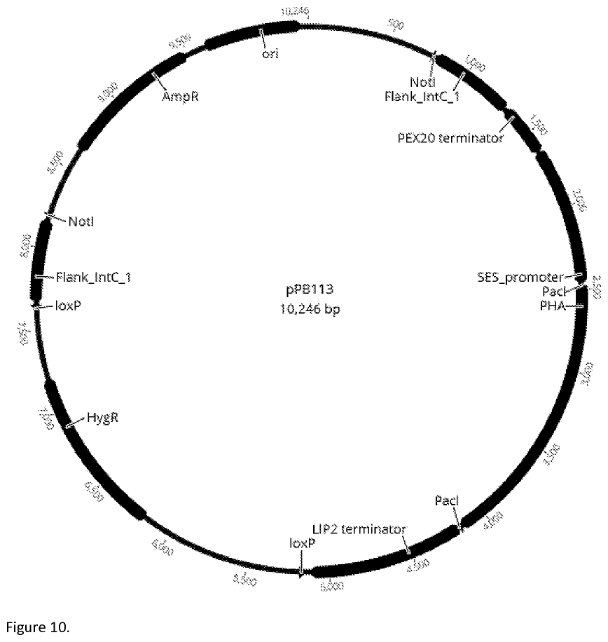

The gene encoding Pseudomonas sp. PHA synthase (SEQ ID NO: 124) amino acid was commercially (Genscript) synthetised with codon optimization for expression in Yarrowia lipolytica cells (SEQ ID NO: 125). The resulting DNA fragment containing coding region of the gene was PCR amplified with oligonucleotides oPlastBug-268 (SEQ ID NO:126, CCTTAATTAAAATGTCCAACAAGAACTC) and pPlastBug-270 (SEQ ID NO:127, GAACAGAAGGAATGCACGCGTTAATTAATTATCGCTCGTGCACGTAG) containing Pacl restriction sites. SES promoter was PCR amplified with oligonucleotides oPlastBug-266 (SEQ ID NO: 128, CAACGGAATGCGTGCGCCGGTGACCTTGGTGGTTC) and oPlastBug-267 (SEQ ID NO: 129, CTTGTTGGACATTTTAATTAAGGAAGCTGATCTGGTG) from plasmid pPB-098-1. Gene fragment and SES promoter was cloned AsiSI digested Easyclone pCfB6577 plasmid (Addgene) with Gibson assembly resulting in plasmid pPB113 (FIG. 10). Notl digested integration fragments were transformed into Yarrowia lipolytica expressing Bacillus flexus superoxide dismutase with Frozen-EZ yeast transformation kit. Transformants having PHA synthase integrated were confirmed with PCR.

Wild type Yarrowia lipolytica (control) and Yarrowia lipolytica strain having PHA synthase and B. flexus superoxide dismutase expressed were cultivated in 50 ml of YPD medium (20 g bacto peptone, 10 g yeast extract, 20 g glucose per litre) overnight. After cultivation cells were harvested 3220 g×10 min and resuspended in 30 ml of water. 3×10 ml aliquots of resuspended samples were centrifuged 3220 g×10 min. One pellet sample per cultivation was stored at −75C (Y sample), second pellet was resuspended in yeast nitrogen base without amino acids (6.7 g per litre) with 0.5% glucose (C sample) and third pellet was resuspended in yeast nitrogen base without amino acids (6.7 g per litre) with 0.5% glucose with PE powder (4000 Da, Sigma) (PE sample). Second and third sample were incubated at +30C with 150 rpm shaking. After 167 hours cultivation cells were harvested and washed with water. Prior lyophilization pellets were stored at −75C. Ten milligram of lyophilized pellet was subjected to methanolysis for 140 min at 100° C. water bath in a solution containing 1 ml chloroform, 20 μl internal standard (butanoic acid), 150 μl sulfuric acid, and 830 μl methanol. Samples were cooled to room temperature and water-soluble particles were removed by addition of 0.5 ml of distilled water. Chloroform phase was analyzed by using gas chromatography system (7890, Agilent) and HP-FFAP column (19091F-102, Agilent). The detected mass range was 35-600 m/z and compounds were identified based on NIST08 MS library.

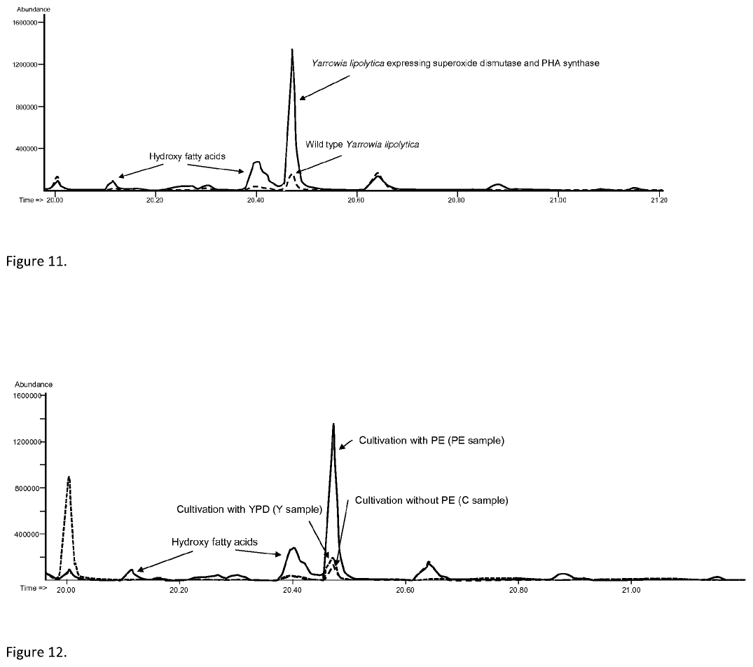

In the GC-MS analysis several hydroxy fatty acids could be detected with the Yarrowia lipolytica strain having superoxide dismutase and PHA synthase expressed which were missing with the wild type Yarrowia lipolytica strain in the cultivations having polyethylene (PE) as a carbon source (PE-samples) (FIG. 11). Additionally, with the same strains these hydroxy fatty acids could be seen only in the cultivation having PE as a carbon source (PE-sample) but not in the cultivations without PE (Y and C samples) (FIG. 12). This indicates that hydroxy fatty acids are originating from PE and are degradation products of polyhydroxyalkanoates. Hydroxy fatty acids are usually intermediates in fatty acid degradation pathway β-oxidation and their amount in the cell is very low or undetectable as seen in FIG. 11 with the wild type Yarrowia lipolytica strain.

### Example 8. PHA Production in Co-Cultivation with Yarrowia lipolytica-Yeast Expressing Bacillus flexus Superoxide Dismutase, Yarrowia lipolytica-Yeast Expressing Bacillus cereus Chloroperoxidase and with Pseudomonas Putida-Bacterium

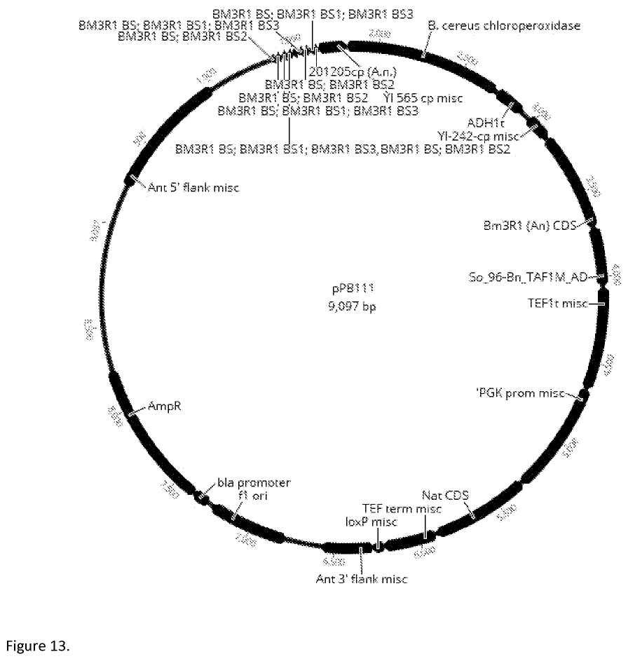

The gene encoding Bacillus cereus chloroperoxidase (SEQ ID NO: 130) amino acid with Yarrowia lipolytica LIP2 signal peptide was commercially (Genscript) synthetized with codon optimization for expression in Yarrowia lipolytica cells (SEQ ID NO: 131). Pacl and Bg/II restriction sites were included at 5′ and 3′ ends of construct for restriction digestion cloning. The constructs were cloned into Yarrowia lipolytica integration cassette plasmid B11157 digested with Pacl and Bc/I. B11157 plasmid contains flanks to ANT1 gene and SES promoter (SES promoter described in Rantasalo et al 2018. Nucleic Acids Research, Volume 46, Issue 18, 12 Oct. 2018, Page e111, https://doi.org/10.1093/nar/gky558). The resulting plasmid was named as pPB111 (FIG. 13). Notl digested integration fragment was transformed into VTT-C-00365 Yarrowia lipolytica strain (VTTCC) with Frozen-EZ yeast transformation kit.

Yarrowia lipolytica strain expressing B. flexus superoxide dismutase (Example 7) was cultivated with Yarrowia lipolytica expressing B. cereus chloroperoxidase (this Example) and PHA producing Pseudomonas putida-bacterium. Yeasts were precultured two days in 50 ml of YPD (20 g Bacto Peptone, 10 g Yeast Extract and 20 g glucose per litre) and P. putida 2 days in LB medium (5 g Yeast Extract, 10 g Tryptone and 10 g NaCl per litre). Five ml of each culture was inoculated in 100 ml shake flask and 2 ml of 10×YNB (yeast nitrogen base without amino acids) was added and volume adjusted to 20 ml with sterile water. Additionally, polyethylene powder (PE, 4 kDa, Sigma-Aldrich) was added. Negative control was without polyethylene. Cultivation at +30° C. with 200 rpm shaking was continued 10 days. After cultivation cells were harvested and washed with water. After washing cells were cold-dried. PHA monomers were identified with methanolysis as described in Example 7. PHA monomer amounts were compared to the cell dry weight (Table 1.)

TABLE 1
PHA monomers amount (μg) per dry cell biomass (mg)

| | 3-OH-decanoic | 3-OH-dodecanoic |
|---|---|---|
| | acid | acid |
| Strains | (μg/mg CDW) | (μg/mg CDW) |
| Yarrrowia with superoxide | 0.00 | 0.31 |
| dismutase | | |
| Pseudomonas putida | | |
| PE plastic | | |
| Yarrrowia with superoxide | 0.01 | 0.40 |
| dismutase | | |
| Yarrowia with chloroperoxidase | | |
| Pseudomonas putida | | |
| PE plastic | | |
| Yarrowia with superoxide | 0.00 | 0.06 |
| dismutase | | |
| Yarrowia with chloroperoxidase | | |
| Pseudomonas putida | | |

This example shows that plastic degradation carried out with superoxide dismutase was enhanced by chloroperoxidase to produce degradation products. This example also shows that PHA producing Pseudomonas putida can use said degradation products in medium chain length PHA production.

1. A method of degrading a hydrocarbon chain, said method comprising:
providing a material comprising a hydrocarbon chain and an enzyme or a fragment thereof capable of degrading the hydrocarbon chain or a polyolefin, and
allowing said enzyme or fragment thereof to degrade the hydrocarbon chain or the polyolefin,
wherein the enzyme or fragment thereof comprises amino acids His31, Tyr35, Glu167, His168, and Tyr171 corresponding to amino acid positions presented in SEQ ID NO: 2, and optionally at least one of the amino acids selected from the group comprising the amino acids His 27, His32, Asn40 and Trp166 corresponding to the amino acid positions presented in SEQ ID NO: 2, and wherein the enzyme is optionally genetically modified.

2. An isolated enzyme or a fragment thereof comprising the amino acids His31, Tyr35, Glu167, His168, and Tyr171 corresponding to the amino acid positions presented in SEQ ID NO: 2, and optionally at least one of the amino acids selected from the group comprising the amino acids His 27, His32, Asn40 and Trp166 corresponding to the amino acid positions presented in SEQ ID NO: 2, wherein said enzyme or fragment thereof is capable of degrading a hydrocarbon chain, and wherein the enzyme is optionally genetically modified.

3. A micro-organism or a host cell comprising an enzyme or a fragment thereof comprising the amino acids His31, Tyr35, Glu167, His168, and Tyr171 corresponding to the amino acid positions presented in SEQ ID NO: 2, and optionally at least one of the amino acids selected from the group comprising the amino acids His 27, His32, Asn40 and Trp166 corresponding to the amino acid positions presented in SEQ ID NO: 2, wherein said enzyme or fragment thereof is capable of degrading a hydrocarbon chain or a polyolefin, and wherein the enzyme is optionally genetically modified.

4. (canceled)

5. The method of claim 1, wherein the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, and the enzyme optionally originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

6. The isolated enzyme or fragment thereof of claim 2, wherein the enzyme or fragment thereof wherein the enzyme or the fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, and wherein the enzyme optionally originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.

7. The method of claim 1, wherein the enzyme is selected from the group comprising or consisting of superoxide dismutases.

8. The method of claim 1, wherein the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), polypropylene (PP), polymethylpentene (PMP), polybutene-1 (PB-1), polyisobutylene (PIB), or any combination thereof; or wherein the enzyme or a fragment thereof is capable of degrading a polyethylene and/or a polypropylene or any combination thereof, and
wherein the length of the hydrocarbon chain optionally is at least C10, C12, C14, C16, C18, C20, C22, C24, C26, C28, C30, C32, C34, C36, C38, C40, C45, C50, C60, C70, C80, C90, C100, C150, C200, C250, C300, C350, C400, C450 or C500.

9. The isolated enzyme or fragment thereof, of claim 2, wherein the hydrocarbon chain is a hydrocarbon chain of a synthetic polymer, alkane, alkene, alkyne, cycloalkane, alkadiene, ketone, fatty acid, alcohol, aldehyde, polyolefin, polyethylene (PE), cross-linked polyethylene (PEX or XLPE), ultra-high molecular weight polyethylene (UHMWPE), high-density polyethylene (HDPE), medium-density polyethylene (MDPE), linear low-density polyethylene (LLDPE), low density polyethylene (LDPE), very low density polyethylene (VLDPE), polypropylene (PP), polymethylpentene (PMP), polybutene-1 (PB-1), polyisobutylene (PIB), or any combination thereof; or wherein the enzyme or a fragment thereof is capable of degrading a polyethylene and/or a polypropylene or any combination thereof, and wherein the length of the hydrocarbon chain is optionally at least C10, C12, C14, C16, C18, C20, C22, C24, C26, C28, C30, C32, C34, C36, C38, C40, C45, C50, C60, C70, C80, C90, C100, C150, C200, C250, C300, C350, C400, C450 or C500.

10. The method according to claim 1, wherein the enzyme or fragment thereof is capable of degrading the hydrocarbon chain or a polyolefin at a temperature of at least 20° C., at least 25° C., at least 30° C., at least 37° C. or at a temperature of 30-95° C., optionally in the presence of a hydrogen peroxide using enzyme, optionally capable of degrading a hydrocarbon chain.

11. The of enzyme or fragment thereof according to claim 2, wherein the enzyme or fragment thereof is capable of degrading the hydrocarbon chain or a polyolefin at a temperature of at least 20° C., at least 25° C., at least 30° C., at least 37° C. or at a temperature of 30-95° C., optionally in the presence of a hydrogen peroxide using enzyme, optionally capable of degrading a hydrocarbon chain.

12. The method of claim 10, wherein the hydrogen peroxide using enzyme is an unspecific peroxygenase (UPO) and/or a chloroperoxidase.

13. The method, of claim 1, wherein at least one or more degradation products selected from the group consisting of an alkane, alkene, alkyne, cycloalkane, alkadiene, ketone, fatty acid, alcohol, aldehyde, epoxy, benzene, styrene, diacid, dione, 2-decanone, 2-dodecanone, 2-tetradecanone, 2-hexadecanone, 2-heptadecanone, 2-dotriacontanone, 2.9-decanedione and 2.11-dodecanedione, are obtained or obtainable by the degradation of the hydrocarbon chain or the polyolefin.

14. (canceled)

15. The method, of claim 1, wherein the enzyme or the fragment thereof, has an increased ability to degrade the hydrocarbon chain or the polyolefin compared to a corresponding unmodified enzyme or fragment thereof.

16. The method of claim 1, wherein the enzyme or fragment thereof comprises a signal sequence, or does not comprise a detectable signal sequence, and wherein the enzyme or fragment thereof is secreted out of the cell, or is not secreted out of the cell.

17. The method of claim 1, wherein the enzyme or fragment thereof is encoded by a heterologous polynucleotide sequence and optionally expressed by a micro-organism or host cell.

18. The micro-organism or host cell of claim 3, wherein the micro-organism or host cell is selected from the group consisting of Escherichia coli, Saccharomyces cerevisiae, Yarrowia lipolytica, Pichia pastoris, Trichoderma reesei, Aspergillus nidulans, Aspergillus niger, Bacillus licheniformis, Bacillus subtilis, and Myceliophthora thermophila.

19. A polynucleotide encoding the enzyme or fragment thereof of claim 2.

20. An expression vector or plasmid comprising the polynucleotide of claim 19.

21. (canceled)

22. A method of producing the enzyme or fragment thereof of claim 2, wherein a recombinant micro-organism or host cell comprising the polynucleotide encoding the enzyme or fragment thereof is allowed to express said enzyme or fragment thereof.

23. A method of producing hydroxy fatty acids, diacids and/or polyhydroxyalkanoate (PHA) from enzymatic degradation products of hydrocarbons by the enzyme of claim 2 or by a -micro-organism and/or host cell comprising the enzyme as substrates to a hydroxy fatty acids, diacids and/or polyhydroxyalkanoate (PHA) producing enzyme, or to a micro-organism and/or a host cell comprising the hydroxy fatty acids, diacids and/or polyhydroxyalkanoate (PHA) producing enzyme.

24. (canceled)

25. The method of claim 23, wherein the host cell producing polyhydroxyalkanoate (PHA) is modified to overexpress an enzyme producing PHA, optionally PHA syntethase.

26. (canceled)

27. The micro-organism or host cell of claim 3, wherein the enzyme or fragment thereof has at least 20, 25, 30, 35, 40, 45, 50, 55, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 2, 4, 6, 8, or 10, and wherein the enzyme optionally originates from or is an enzyme of a bacterium or fungus selected from the group comprising or consisting of Bacillus, Paenibacillus, Achromobacter, Acinetobacter, Alcanivorax, Aneurinibacillus, Arthrobacter, Aspergillus, Brevibacillus, Brucella, Chitinophaga, Citrobacter, Comamonas, Cordyceps, Cupriavidus, Delftia, Enterobacter, Escherichia, Exiguobacterium, Flavobacterium, Fusarium, Halomonas, Hyphomicrobium, Klebsiella, Kocuria, Leucobacter, Lysinibacillus, Macrococcus, Methylobacterium, Methylocella, Microbacterium, Micrococcus, Moraxella, Mucor, Nesiotobacter, Nocardia, Ochrobactrum, Pantoea, Paracoccus, Penicillium, Pleurotus, Pseudomonas, Rahnella, Ralstonia, Rhizobium, Rhodococcus, Serratia, Sphingobacterium, Staphylococcus, Stenotrophomonas, Streptococcus, Streptomyces, Trichoderma, Vibrio, Virgibacillus and Xanthobacter; or the enzyme is an enzyme of a bacterium or fungus selected from the group comprising or consisting of Achromobacter xylosoxidans, Acinetobacter sp., Acinetobacter baumannii, Acinetobacter pittii, Alcanivorax borkumensis, Aneurinibacillus aneurinilyticus, Arthrobacter sp, Aspergillus awamori, Aspergillus flavus, Aspergillus fumigatus, Aspergillus glaucus, Aspergillus niger, Aspergillus oryzae, Aspergillus sp. Aspergillus sydowii, Aspergillus terreus, Bacillus amyloliquefaciens, Bacillus licheniformis, Bacillus mycoides, Bacillus pumilus, Bacillus sp., Bacillus subtilis, Bacillus cereus, Bacillus flexus, Bacillus cohnii, Bacillus circulans, Bacillus thuringiensis, Bacillus aryabhattai, Bacillus gottheilii, Bacillus vallismortis, Bacillus vietnamensis, Brevibacillus brevis, Brevibacillus borstelensis, Brevibacillus agri, Brevibacillus parabrevis, Brevibacillus sp., Brucella anthropi, Chitinophaga sp., Citrobacter amalonaticus, Comamonas sp., Cordyceps confragosa, Cupriavidus necator, Delftia sp., Delftia tsuruhatensis, Enterobacter asburiae, Enterobacter sp., Escherichia coli, Exiguobacterium sp. Flavobacterium sp., Flavobacterium petrolei, Flavobacterium pectinovorum, Flavobacterium aquicola, Fusarium solani, Fusarium sp., Halomonas venusta, Hyphomicrobium sp., Klebsiella pneumoniae, Kocuria palustris, Leucobacter sp., Lysinibacillus fusiformis, Lysinibacillus sphaericus, Lysinibacillus xylanilyticus, Lysinibacillus halotolerans, Macrococcus caseolyticus, Methylobacterium aquaticum, Methylobacterium indicum, Methylocella silvestris, Microbacterium sp., Microbacterium paraoxydans, Micrococcus sp., Micrococcus luteus, Micrococcus lylae, Moraxella sp., Mucor circinelloides, Nesiotobacter exalbescens, Nocardia asteroides, Ochrobactrum intermedium, Ochrobactrum oryzae, Paenibacillus sp., Paenibacillus odorifer, Paenibacillus macerans, Pantoea sp., Paracoccus yeei, Penicillium chrysogenum, Penicillium oxalicum, Penicillium ostreatus, Pseudomonas aeruginosa, Pseudomonas azotoformans, Pseudomonas chlororaphis, Pseudomonas citronellolis, Pseudomonas fluorescens, Pseudomonas monteilii, Pseudomonas protegens, Pseudomonas putida, Pseudomonas sp., Pseudomonas stutzeri, Pseudomonas syringae, Rahnella aquatilis, Ralstonia sp., Rhizobium viscosum, Rhodococcus ruber, Rhodococcus gingshengii, Rhodococcus erythropolis, Rhodococcus rhodochrous, Rhodococcus sp., Serratia marcescens, Sphingobacterium multivorum, Staphylococcus epidermidis, Staphylococcus cohnii, Staphylococcus sp., Staphylococcus xylosus, Stenotrophomonas humi, Stenotrophomonas maltophilia, Stenotrophomonas panacihumi, Stenotrophomonas sp., Streptococcus sp., Streptomyces albogriseolus, Streptomyces badius, Streptomyces griseus, Streptomyces sp., Streptomyces viridosporus, Trichoderma harzianum, Trichoderma virens, Vibrio alginolyticus, Vibrio parahaemolyticus, Virgibacillus halodenitrificans, Xanthobacter autotrophicus, and Xanthobacter tagetidis.


SEQUENCE LISTING (131 sequences)

SEQ ID NO: 1 - DNA, 609 bp, Bacillus licheniformis
  source 1..609: mol_type = genomic DNA
atggcttacgaacttccagaattaccttatgcgtacgatgctttagaaccgcatatcgacaaggaaactatgactattcaccatacgaaacaccacaacacatacgtgacaaatttaaacaaagcggttgaaggaaacactgctcttgccaacaaatcagttgaagagcttgttgcagatcttgattctgttcctgaaaacatccgcactgcagtccgcaacaacggcggcggacacgcgaaccacaaattattctggacacttttatctccaaacggcggaggcgaaccgactggcgcgcttgctgaagagatcaacagtgtatttggaagctttgacaaattcaaagagcaattcgcagcagcagcagctggacgcttcggttctggctgggcatggcttgttgtgaacaacggtaaacttgaaatcacaagcacgccaaaccaagattctccgctttcagaaggcaaaacgcctatcttaggtcttgacgtttgggagcatgcttactaccttaactatcaaaaccgccgtcctgattacatttcagctttctggaatgttgtaaactgggacgaagtggctcgtctatacagcgaagcaaaataa

SEQ ID NO: 2 - protein, 202 aa, Bacillus licheniformis
MAYELPELPYAYDALEPHIDKETMNIHHTKHHNTYVTKLNEAVAGKQDLESKSVEELVANLDAVPENIRTAVRNNGGGHANHSLFWKLLSPNGGGAPTGELAEAINSKFGSFDQFKEDFAAAAAGRFGSGWAWLVVNNGELEITSTPNQDSPLSEGKTPILGLDVWEHAYYLNYQNRRPDYIKAFWNVVNWDEVARLYSEAK

SEQ ID NO: 3 - DNA, 612 bp, Bacillus cereus
  source 1..612: mol_type = genomic DNA
atggcaaaacacgaattaccaaatttaccttatgcgtatgatgctttagagcctcactttgacaaagaaacaatgaacatccatcatacaaagcaccataacacgtacatcacaaacttaaacgctgctttagaaggtcatgcagaattagctgacaaaagcgtagaagaattagttgcaaacttaaacgaagtaccagaagcaatccgtacagcagtacgtaacaatggtggtggacatgctaaccacacattcttctggacaatcttatctccaaacggcggcggacaaccagtaggcgaacttgcaactgcaattgaagcgaaattcggtagcttcgatgcattcaaagaagaattcgcaaaagctggcgcaactcgcttcggttctggttgggcttggttagtagtaaataatggtgagttagaagtaacaagcacgccaaaccaagattctcctctaactgaaggtaaaactccagttatcggtttagatgtttgggaacatgcttactacttaaattatcaaaaccgtcgtccagactacatcggtgcattctggaacgttgtagattggaacgctgctgaaaaacgttaccaagaagcaaaataa

SEQ ID NO: 4 - protein, 203 aa, Bacillus cereus
MAKHELPNLPYAYDALEPHFDKETMNIHHTKHHNTYITNLNAALEGHAELADKSVEELVANLNEVPEAIRTAVRNNGGGHANHTFFWTILSPNGGGQPVGELATAIEAKFGSFDAFKEEFAKAGATRFGSGWAWLVVNNGELEVTSTPNQDSPLTEGKTPVIGLDVWEHAYYLNYQNRRPDYIGAFWNVVDWNAAEKRYQEAK

SEQ ID NO: 5 - DNA, 609 bp, Bacillus flexus
  source 1..609: mol_type = genomic DNA
atggcttacgaattaccacaattaccttatgcatatgatgcacttgaaccacacatcgacaaagaaacgatgaacattcaccatacgaaacaccataacacttacgttacaaacttaaacgcagcagtagaaggaaaagctgatttagcttctaaaagtgttgaagagttaatctcaaatatggatgctgtaccagaagatatccgtacagcagtacgtaacaatggtggtggacatgctaaccactcattattctggacaattctttgccctaacggtggcggcgagccaacaggcgagttagcagacgcaattagcagtaagtttggaagctttgacgcattcaaagatgcattcgctaaagcagcaactacacgcttcggttctggctgggcttggttagttgtaaacaatggtgaattagaagtaacaagtacaccaaaccaagatagcccactaatggaaggtaaaacaccaatcctaggactagacgtttgggagcatgcttactacttaaactaccaaaaccgtcgtccagattacatttcagcattctttaatgtaattaaatgggacgaagttgcaaaacgctacgcagcagcaaaataa

SEQ ID NO: 6 - protein, 202 aa, Bacillus flexus
MAYELPQLPYAYDALEPHIDKETMNIHHTKHHNTYVTNLNAAVEGKADLASKSVEELISNMDAVPEDIRTAVRNNGGGHANHSLFWTILCPNGGGEPTGELADAISSKFGSFDAFKDAFAKAATTRFGSGWAWLVVNNGELEVTSTPNQDSPLMEGKTPILGLDVWEHAYYLNYQNRRPDYISAFFNVIKWDEVAKRYAAAK

SEQ ID NO: 7 - DNA, 609 bp, Bacillus subtilis; identical to SEQ ID NO: 1
  source 1..609: mol_type = genomic DNA

SEQ ID NO: 8 - protein, 202 aa, Bacillus subtilis
MAYELPELPYAYDALEPHIDKETMTIHHTKHHNTYVTNLNKAVEGNTALANKSVEELVADLDSVPENIRTAVRNNGGGHANHKLFWTLLSPNGGGEPTGALAEEINSVFGSFDKFKEQFAAAAAGRFGSGWAWLVVNNGKLEITSTPNQDSPLSEGKTPILGLDVWEHAYYLNYQNRRPDYISAFWNVVNWDEVARLYSEAK

SEQ ID NO: 9 - DNA, 624 bp, Rhodococcus ruber
  source 1..624: mol_type = genomic DNA
atggctgagtacacacttccggacctgccctacgactacgccgcgctcgagccgcacatctcggcgaagatcatggagctgcaccacagcaagcatcacgcgacctacgttgccggcgccaacaccgccctcgagaagatggcggaggcacgggagaacggcacgatggcggcgcaggccaccaagctggagaaggacctggccttccacctcggcggccacaccaaccactccgtcttctggaagaacctgtcgccggagggtggcgacaagccggacggtgaactcggtgccgcgatcgacgacgcgttcggttccttcgacgccttccgggaccacttctcggcgaacgccaactcgatccagggctccggctggtccatcctggcctgggactccatcggccagcgcctgatcatcgtccagctgtacgaccagcagggcaacatcccgatcggtctgaccccgctgctcatgctcgacatgtgggagcacgccttctacctgcagtaccagaacgtcaaggccgactacgtcaaggccttctggaacatcgtcaactgggccgacgtgcaggcccgcttcgagcgcgcccgcacccagacctccggtctgctggtctag

SEQ ID NO: 10 - protein, 207 aa, Rhodococcus ruber
MAEYTLPDLPYDYAALEPHISAKIMELHHSKHHATYVAGANTALEKMAEARENGTMAAQATKLEKDLAFHLGGHTNHSVFWKNLSPEGGDKPDGELGAAIDDAFGSFDAFRDHFSANANSIQGSGWSILAWDSIGQRLIIVQLYDQQGNIPIGLTPLLMLDMWEHAFYLQYQNVKADYVKAFWNIVNWADVQARFERARTQTSGLLV

SEQ ID NO: 11 - DNA, 73 bp, synthetic construct
  misc_feature 1..73: note = oPlastBug-242 oligonucleotide
  source 1..73: mol_type = other DNA
tagaaataattttgtttaactttaagaaggagatatatccatggcttacaaacttccagaattaccttatgct

SEQ ID NO: 12 - DNA, 67 bp, synthetic construct
  misc_feature 1..67: note = oPlastBug-243 oligonucleotide
  source 1..67: mol_type = other DNA
caagctgggatttaggtgacactatagaatactcaagcttttattttgcttcgctgtaaaggcgtgc

SEQ ID NO: 13 - DNA, 72 bp, synthetic construct
  misc_feature 1..72: note = oPlastBug-238 oligonucleotide
  source 1..72: mol_type = other DNA
tagaaataattttgtttaactttaagaaggagatatatccatgtcacttaagtggcaatacataaactggga

SEQ ID NO: 14 - DNA, 73 bp, synthetic construct
  misc_feature 1..73: note = oPlastBug-239 oligonucleotide
  source 1..73: mol_type = other DNA
caagctgggatttaggtgacactatagaatactcaagcttttattttgcttcttggtaacgtttttcagcagc

SEQ ID NO: 15 - DNA, 83 bp, synthetic construct
  misc_feature 1..83: note = oPlastBug-138 oligonucleotide
  source 1..83: mol_type = other DNA
acaattcctctagaaataattttgtttaactttaagaaggagatatatccatggcttacgaattaccacaattaccttatgca

SEQ ID NO: 16 - DNA, 77 bp, synthetic construct
  misc_feature 1..77: note = oPlastBug-139 oligonucleotide
  source 1..77: mol_type = other DNA
ttgttagcagccggatcaagctgggatttaggtgacactatagaatactcttattttgctgctgcgtagcgttttgc

SEQ ID NO: 17 - DNA, 72 bp, synthetic construct
  misc_feature 1..72: note = oPlastBug-244 oligonucleotide
  source 1..72: mol_type = other DNA
tagaaataattttgtttaactttaagaaggagatatatccatggcttacgaacttccagaattaccttatgc

SEQ ID NO: 18 - DNA, 69 bp, synthetic construct
  misc_feature 1..69: note = oPlastBug-245 oligonucleotide
  source 1..69: mol_type = other DNA
caagctgggatttaggtgacactatagaatactcaagcttttattttgcttcgctgtatagacgagcca

SEQ ID NO: 19 - DNA, 75 bp, synthetic construct
  misc_feature 1..75: note = oPlastBug-136 oligonucleotide
  source 1..75: mol_type = other DNA
acaattcctctagaaataattttgtttaactttaagaaggagatatatccatggctgagtacacacttccggacc

SEQ ID NO: 20 - DNA, 73 bp, synthetic construct
  misc_feature 1..73: note = oPlastBug-137 oligonucleotide
  source 1..73: mol_type = other DNA
ttgttagcagccggatcaagctgggatttaggtgacactatagaatactcctagaccagcagaccggaggtct

SEQ ID NO: 21 - protein, 234 aa, synthetic construct
MKLSTILFTACATIAAALPSPITPSEAAVLQKRAYELPQLPYAYDALEPHIDKETMNIHHTKHHNTYVTNLNAAVEGKADLANKSVEELISNMDAVPEDIRTAVRNNGGGHANHKLFWTILCPNGGGEPTGELADAISSKFGSFDAFKDAFAKAATTRFGSGWAWLVVNNGELEVTSTPNQDSPLMEGKTPILGLDVWEHAYYLNYQNRRPDYISAFFNVIKWDEVAKRYAAAK

SEQ ID NO: 22 - DNA, 705 bp, synthetic construct
  misc_feature 1..705: note = Bacillus flexus superoxide dismutase with Ser51Asn and Ser83Lysmutations and with Yarrowia lipolytica LIP2 signal peptide codonoptimised to Yarrowia lipolyt
  source 1..705: mol_type = other DNA
atgaagctgtctaccatcctcttcaccgcctgtgctaccatcgccgctgccctgccttctcccatcactccctctgaggccgctgtcctgcagaagcgagcctacgagctgccccagctgccttacgcctacgacgctctcgagccccatattgacaaggagaccatgaacatccaccataccaagcaccataacacctacgtgaccaacctcaacgccgctgttgagggaaaggctgacctggccaacaagtctgtggaggagctgatctccaacatggacgccgtgcccgaggacattcgaaccgccgtgcgaaacaacggtggaggtcacgctaaccacaagctgttctggaccattctgtgccctaacggaggtggcgagcctaccggagagctggccgacgctatttcctctaagtttggatctttcgacgctttcaaggacgctttcgccaaggctgccaccactcgattcggttctggatgggcttggctcgttgtgaacaacggcgagctggaggtgacctctactcctaaccaggattctcctctgatggagggcaagacccctattctgggcctggacgtgtgggagcacgcctactacctgaactaccagaaccgacgacccgactacatttccgccttcttcaacgtgatcaagtgggatgaggttgctaagcgatacgccgctgctaagtaa

SEQ ID NO: 23 - protein, 202 aa, Bacillus cohnii
MAYELPQLPYAYDALEPHIDKETMNIHHTKHHNTYVTNVNAALEGHADLQSKSVEELISNLDAVPENIRTAVRNNGGGHANHSLFWTLLTPGGSNAPTGELADAINSKFGSLESFKEEFTKAATTRFGSGWAWLVVNNGELEVTSTPNQDSPLMEGKTPVLGLDVWEHAYYLNYQNRRPDYISAFFNVINWDVVGELYGKAK

SEQ ID NO: 24 - protein, 204 aa, Achromobacter xylosoxidans
MAYTLPPLPYTYDALEPHIDAMTMEIHYSKHHQTYVNSLNAALEGAGIATDEPVESLVARLEQLPEAVRGAVRNHGGGHANHSLFWAVMSPEGGGEPDGALAAVIEAELGGLAAFRDAFTKAALTRFGSGWAWLSVTPAGKLTVESSANQDSPLMQGNTPILGLDVWEHAYYLKYQNRRPEYIGAFYNVVNWPEVARRYAAAQR

SEQ ID NO: 25 - protein, 208 aa, Acinetobacter baumannii
MTTITLPALPYGYDDLAPHISKETLEYHHDKHHNTYVVNLNNLIKGTDLEGKTLEEIIKATAGDASKAGIFNNAAQVWNHTFYWNSMKPNGGGKPTGAIAAKIDEAFGSYEKFAEEFTAAATTQFGSGWAWLVADEVNGKLSITKTSNADTPLAHGQIAVLTIDVWEHAYYIDFRNLRPKYIATFLENLVNWDYANAKLAGQPAGVEK

SEQ ID NO: 26 - protein, 208 aa, Acinetobacter pittii
MTTITLPALPYGYDDLAPHISKETLEYHHDKHHNTYVVNLNNLIKGTDLEGKTLEEIIKATAGDASKAGIFNNAAQVWNHTFYWNSMKPNGGGKPTGAIAAKIDEAFGSYEKFAEEFTAAATTQFGSGWAWLVADEVNGKLSITKTSNADTPLAHGQIAVLTIDVWEHAYYIDFRNLRPKYIATFLESLVNWDYANAKLAGQPAGVEK

SEQ ID NO: 27 - protein, 195 aa, Alkanivorax borkumensis
MAFELPPLPFEKNALEPHISAETLEYHHGKHHNAYVTKLNDMVAGTDNEGKSLEEIIKSAEGGLFNQAAQVWNHTFYWNSLSPNGGGAPSGDLGAAIDKAFGSFDEFKDQFNAKATGNFGSGWTWLVKNSDGSLEIVNTDDADTPIAHGKGQTPLFTVDVWEHAYYVDYRNARPKYLESIWNLINWDFAASNFAA

SEQ ID NO: 28 - protein, 203 aa, Aneurinibacillus aneurinilyticus
MAKFELPALPYSFDALEPHIDAQTMEIHHDRHHATYVNNLNAALEGQAELADKNIEDLMRNINSVPENIRTAVRNNGGGHYNHSLFWEIMSPNGGGEPTGDVATAINESFGSFDKLKEEFAKAAATRFGSGWAWVIVDNGKLAITSTPNQDNPLMEGKTPILGLDVWEHAYYLKYQNKRPDYISAWWNTVNWDAVNKRFNEAK

SEQ ID NO: 29 - protein, 207 aa, Arthobacter sp.
MTEYVLPELGYDYAALEPHISAKIMELHHSKHHAAYVAGANNALAQLAEAREKGDFANINRLSKDLAFHTGGHINHSVFWNNISPDGGDKPEGELAAAIDDAFGSFDAFRAQFTAAALGLQGSGWAFLAYEPIAGNLLIEQLYDQQGNVAVGTTPLLMLDMWEHAFYLDYVNVKADYVKAFWNIVNWADVAKRFEAARANATGLITL

SEQ ID NO: 30 - protein, 231 aa, Aspergillus awamori
MAASLVRTSARTALRAGASATPRTAGMAGLTFARGKATLPDLSYDYGALEPSISGKIMELHHKNHHQTYVNSYNTAIEQLQEAQHKNDIAAQIALKPLINFHGGGHLNHTLFWENLAPKSAGGGEPPSGALSTAINDTFGSLEEFQNKMNAALAAIQGSGWAWLVKDKQTGHIGIKAYANQDPVVGQFQPLLGIDAWEHAYYLQYQNRKAEYFKAIWEVINWKAVEKRFSA

SEQ ID NO: 31 - protein, 230 aa, Aspergillus flavus
MAASLIRTSARTALRAGASATPKAAGVAGLTFARGKATLPDLAYDYGALEPSISGKIMELHHKNHHQTYVNSYNTAIEQLQEAVAKEDITTQINLKPLINFHGGGHINHTLFWENLAPKSQGGGEPPSGALAKAIDESFGSLGEFQSKMNAALAGIQGSGWAWLVKDKQTGNIGIKTYANQDPVVGQFQPLLGIDAWEHAYYLQYQNRKAEYFSAIWDVINWKAVEKRFS

SEQ ID NO: 32 - protein, 210 aa, Aspergillus fumigatus
MSQQYTLPPLPYPYDALQPYISQQIMELHHKKHHQTYVNGLNAALEAQKKAAEANDVPKLVSVQQAIKFNGGGHINHSLFWKNLAPEKSGGGKIDQAPVLKAAIEQRWGSFDKFKDAFNTTLLGIQGSGWGWLVTDGPKGKLDITTTHDQDPVTGAAPVFGVDMWEHAYYLQYLNDKASYAKGIWNVINWAEAENRYIAGDKGGHPFMKL

SEQ ID NO: 33 - protein, 208 aa, Aspergillus glaucus
MSQFTLPPLPYAYDALEPVISKQIMEIHHQKHHQTYITNLNAALTAQSTALQSQDVPQLISLQQKIKFNGGGHINHSLFWKNLTPASSPQAKLESAPGVKAAIERKWGSYEGFQKVFSATLLAIQGSGWGWLVANKPKGELEIVTTKDQDPVTGDTPLFGVDMWEHAYYLQYLNNKASYVDGIWKIINWSTTEDRYQNGIEGPAVLKL

SEQ ID NO: 34 - protein, 231 aa, Aspergillus niger; identical to SEQ ID NO: 30

SEQ ID NO: 35 - protein, 230 aa, Aspergillus oryzae; identical to SEQ ID NO: 31

SEQ ID NO: 36 - protein, 228 aa, Aspergillus sp.
MATSLFRSSARSALRVGASATSKGLSGMTFARGKATLPDLSYDYGALEPSISGKIMEVHHKAHHQTYVNNYNTAVEQIQQAQSSNDIAAQIALKPAINFNGGGHLNHTLFWENLAPKSAGGGEPPSGSLSQAINTTYGSLDEFKNKMNTALAGIQGSGWAWLVKDKNTGQIGIRTYANQDPVVGQFAPLLGIDAWEHAYYLQYQNRKAEYFKSIWEVINWKAVEARFS

SEQ ID NO: 37 - protein, 224 aa, Aspergillus sydowii
MASLIRTSLRTGLRASSSSAASSLTFTRGKATLPDLAYDYGALEPAVSGKIMELHHKNHHQTYVNSYNTALEQLQEAQAKNDISAQIALKPLINFHGGGHINHTLFWENLAPKSAGGGEPPSGALSKAINESFGSLENFQGQMNTALAGIQGSGWAWLVQDKQTGNIAIKTYANQDPVVGQFKPLLGIDAWEHAYYLQYQNRKAEYFKAIWDVINWKNVEKRFS

SEQ ID NO: 38 - protein, 227 aa, Aspergillus terreus
MAASLLRTSARSAFRASATPKAGVASLTFARGKATLPDLAYDYGALEPSISGKIMELHHKNHHQTYVNSYNTAIEQLQEAQAKNDISAQIALKPLINFHGGGHLNHTLFWENLAPKNAGGGEPPSGALAKAIDDSFGSLEEFQNKMNTALAGIQGSGWAWLVKDKQTGNIGIKTYANQDPVVGQFQPLLGIDAWEHAYYLQYQNRKAEYFKAIWEVINWKAVEKRFT

SEQ ID NO: 39 - protein, 202 aa, Bacillus sp.
MAFELPQLPYAEDALEPHIDKETMNIHHTRHHNTYVTNLNNALAGNEELLSKSVEEVISNLDAVPESARTAVRNNGGGHANHSLFWTILSPNGGGAPSGELADAINSKFGSFEAFKEEFAKAATTRFGSGWAWLAVNNGELELTSTPNQDSPLMEGKTPILGLDVWEHAYYLKYQNKRPDYIGAFWNVVNWDEVAKRYSAAK

SEQ ID NO: 40 - protein, 201 aa, Bacillus amyloliquefaciens
MAYKLPELPYAYDALEPHIDKETMTIHHTKHHNTYVTNLNKAIEGSALAEKSVDELVADLNAVPEDIRTAVRNNGGGHANHSLFWTLLSPNGGGEPTGELAEEIKSTFGSFDQFKEKFAAAAAGRFGSGWAWLVVNNGKLEITSTPNQDSPLSEGKTPVLGLDVWEHAYYLNYQNRRPDYISAFWNVVNWDEVARLYSEAK

SEQ ID NO: 41 - protein, 202 aa, Bacillus aryabhattai
MAYKLPELPYAYDALEPHIDKETMNIHHTKHHNTYVTNLNAAVEGKADLESKSIEELISNLDAVPEDIRTAVRNNGGGHANHSLFWTILCPNGGGAPTGELADAIASKFGSFDQFKEEFANAAKTRFGSGWAWLVVNNGELEVTSTPNQDSPLMEGKTPILGLDVWEHAYYLNYQNRRPDYISAFFNVVKWDEVAKRYDAAK

SEQ ID NO: 42 - protein, 203 aa, Bacillus mycoides
MAKHELPNLPYAYDALEPHFDKETMNIHHTKHHNTYITNLNAALEGHAELADKSVEELVANLNEVPEAIRTAVRNNGGGHANHTFFWTILSPNGGGQPVGELATAIEAKFGSFDAFKVEFAKAGATRFGSGWAWLVVNNGELEVTSTPNQDSPITEGKTPVIGLDVWEHAYYLNYQNRRPDYIGAFWNVVDWNVAEKLYQEAK

SEQ ID NO: 43 - protein, 202 aa, Bacillus pumilus
MAFKLPELPYAYDALEPHIDKETMTIHHTKHHNTYVTNLNKAIEGVSALEDQSIDELVANLNSVPENIRTAVRNNGGGHANHSLFWTLLSPNGGGAPTGELADAIEKELGGFEKFKSDFAAAAAGRFGSGWAWLVVNNGKLEITSTPNQDSPLTEGKTPILGLDVWEHAYYLNYQNRRPDYISAFWNVVNWDEVARLYSEAK

SEQ ID NO: 44 - protein, 208 aa, Bacillus thuringiensis
MSSFQLPKLSYDYDELEPHIDSNTLSIHHGKHHATYVNNLNATLENYTELHNKSLEELLCNLDALPKEIVTAVRNNGGGHYCHSLFWEVMSPRGGGEPNGDVAKVIDYYFNTFDNLKDQLSKAAISRFGSGYGWLVLDGEELSVMSTPNQDTPLQEGKIPLLVIDVWEHAYYLKYQNRRPEFVTNWWHTVNWDQVNEKYLQAIQSQKH

SEQ ID NO: 45 - protein, 202 aa, Bacillus vallismortis
MAYELPELPYAYDALEPHIDKETMTIHHTKHHNTYVTNLNKAVEGNTALANKSVEELVADLNSVPENIRTAVRNNGGGHANHKLFWTLLSPNGGGEPTGALADEINSVFGSFDKFKEQFAAAAAGRFGSGWAWLVMNNGKLEITSTPNQDSPLSEGKTPILGLDVWEHAYYLNYQNRRPDYISAFWNVVNWDEVARLYSEAK

SEQ ID NO: 46 - protein, 202 aa, Bacillus vietnamensis
MAFELPQLPYAYDALEPHIDKETMNIHHTKHHNAYVTKLNDALQGNDELLSKSVEEVVANLDAVPEGARTAVRNNGGGHANHSLFWTILSPNGGGSPEGELASAIESKFGSFEKFKEEFAAAGAGRFGSGWAWLVVNNGELEVTSTLNQDSPLMEGKTPVLGLDVWEHAYYLNYQNRRPDYINAFWNVVNWEEVSKRYSAAK

SEQ ID NO: 47 - protein, 202 aa, Brevibacillus agri
MAHQLPALPYAHDALEPHIDAQTMEIHHGRHHATYVNNLNAALEAHPDLQQKSVEELISNLDALPESIRTAVRNNGGGHANHTLFWEILSPNGGGAPTGELADAINAAFGSFDNFKAEFAKAATTRFGSGWAWLTAEGGKVAITSTPNQDSPIMEGKTPILGLDVWEHAYYLRYQNKRPDYISAFWNVVNWDEVSKRFAAAK

SEQ ID NO: 48 - protein, 202 aa, Brevibacillus borstelensis
MAHQLPELPYAHNALEPHIDAQTMEIHHGRHHATYVNNLNAALEGHADLQAKSVEELISNLDALPEAIRTAVRNNGGGHANHALFWQILSPNGGGAPAGALADAINAAFGSFDNFKAEFAKAATTRFGSGWAWLIVDGGKLAITSTPNQDSPIMEGKTPILGLDVWEHAYYLKYQNKRPDYIGAFWNVVNWDEVSKRFEAAR

SEQ ID NO: 49 - protein, 202 aa, Brevibacillus brevis
MAHQLPALPYAHNALEPHIDAQTMEIHHGRHHATYVNNLNAALEAHADLQGKSIEELISNLDALPESIRTAVRNNGGGHANHTLFWEIMSPNGGGAPTGELADAINAAFGSFDNFKAEFAKAATTRFGSGWAWLTAEGGKVAISSTPNQDSPIMEGKTPILGLDVWEHAYYLNYQNKRPDYIGAFWNVVNWDEVSKRFAAAK

SEQ ID NO: 50 - protein, 202 aa, Brevibacillus parabrevis; identical to SEQ ID NO: 47

SEQ ID NO: 51 - protein, 202 aa, Brevibacillus sp.; identical to SEQ ID NO: 47

SEQ ID NO: 52 - protein, 206 aa, Citrobacter amalonaticus
MSYTLPSLPYAYDALEPHFDKQTMEIHHTKHHQTYVNNANAALESLPEFANLSAEELIAKLDQVPADKKTVLRNNAGGHANHSLFWKGLKKGTTLQGDLKAAIERDFGSVDNFKAEFEKAAATRFGSGWAWLVLKGDKLAVVSTANQDSPLMGEAISGASGFPIVGLDVWEHAYYLKFQNRRPDYIKEFWNVVNWDEAAARFAAKK

SEQ ID NO: 53 - protein, 211 aa, Comamonas sp.
MSYTLPPLAYDYAALEPHIDAQTMEIHYCRHHQTYVNNLNAALEGSEYAQWSLDDLLAKVESLPQALRQAVRNNGGGHANHSLFWQVMSPGGGGLPQNALAAAIDKDLGGFENFKTEFTKAAISRFGSGWAWLSVDRSGKLHVESSANQDNPLMLGIGSGNTPILGLDVWEHAYYLKYQNRRPEYIAAFYNVVDWNEVARRFAAATQAVRS

SEQ ID NO: 54 - protein, 230 aa, Cordyceps confragosa
MASALLRTTPAVRAGLRAASKPAAAMASTSFVRGKATLPDLPYDYAALEPAISGKIMELHHSKHHQTYVNGFNAAVEALSEAQAKNDAKAAAAQAPLLNFHGGGHVNHSLFWENLAPNGKGGGGEPAGKLSTAITEDFGSFDNLKKQTNAALAAIQGSGWAWLVKDKTSGTLSVVTRPNQDPIAGNLEPLLGIDAWEHAYYLQYENRKAEYFSAIWDVINWGTVAKRFEK

SEQ ID NO: 55 - protein, 193 aa, Cupriavidus necator
MEHKLPPLPYAHDALAPHISKETLEFHHDKHHQTYVTNLNNLIKGTEFENSTLEEIVKKSSGGIFNNAAQVWNHTFYWDSMKPNGGGQPTGALADAINAKWGSFDKFKEEFTKTAVGTFGSGWAWLVKKTDGSLDLVSTSNAATPLTTDAKALLTCDVWEHAYYIDYRNARPKYVEAFWNVVNWDFAGKNFAG

SEQ ID NO: 56 - protein, 211 aa, Delftia sp.
MSNYTLPPLNYAYDALEPHIDAQTMEIHYTKHHQAYVNNLNAALKDTPHGSQPVEQLIADLSLLPEGVRGAVRNNGGGHANHSLFWTVMAPVDQGGGGAPSGELAQAIDRELGGFDAFKAAFTQAALTRFGSGWAWLVVGKDGKLAVESSGNQDSPLMAGIGSGATPILGLDVWEHAYYLKYQNRRPEYIAAFYNVVNWAEVARRYAAARG

SEQ ID NO: 57 - protein, 211 aa, Delftia tsuruhatensis
MSNYTLPPLNYAYDALEPHIDAQTMEIHYTKHHQAYVNNLNAALKDTPHGSQPVEQLIADLSLLPEGVRGAVRNNGGGHANHSLFWTVMAPTDQGGGGAPSGELAQAIDRELRGFDAFKAAFTLAALTRFGSGWAWLVVGKDGKLAVESSGNQDSPLMAGIGSGATPILGLDVWEHAYYLKYQNRRPEYIAAFYNVVSWAEVARRYAAARG

SEQ ID NO: 58 - protein, 206 aa, Enterobacter sp.
MSYTLPSLPYAYDALEPHFDKQTMEIHHTKHHQAYVNNANAALESLPEFAELSVEELITKLDQLPADKKTVLRNNAGGHANHSLFWKGLKTGTTLQGDLKAAIERDFGSVDAFKAEFEKAAATRFGSGWAWLVLKGDKLAVVSTANQDSPLMGEAVSGVSGFPIVGLDVWEHAYYLKFQNRRPDYIKAFWDVVNWDEAAARFAAKK

SEQ ID NO: 59 - protein, 206 aa, Enterobacter asburiae
MSYTLPSLPYAYDALEPHFDKQTMEIHHTKHHQTYVNNANAALESLPEFANLSAEELITKLDQLPADKKTVLRNNAGGHANHSLFWKGLKTGTTLQGDLKAAIERDFGSVDNFKAEFEKAAATRFGSGWAWLVLKGDKLAVVSTANQDSPLMGEAISGASGFPILGLDVWEHAYYLKFQNRRPDYIKAFWDVVNWDEAAARFAAKK

SEQ ID NO: 60 - protein, 206 aa, Escherichia coli
MSYTLPSLPYAYDALEPHFDKQTMEIHHTKHHQTYVNNANAALESLPEFANLPVEELITKLDQLPADKKTVLRNNAGGHANHSLFWKGLKKGTTLQGDLKAAIERDFGSVDNFKAEFEKAAASRFGSGWAWLVLKGDKLAVVSTANQDSPLMGEAISGASGFPIMGLDVWEHAYYLKFQNRRPDYIKEFWNVVNWDEAAARFAAKK

SEQ ID NO: 61 - protein, 202 aa, Flavobacterium sp.
MAFELPQLPYAYDALEPYIDAKTMEIHHSKHHNAYTTNLNAAIAGTDLEGLTIENILINLDMKNAAVRNNGGGFYNHNLFWRVMAPNGGGLPTGELAEAIERDFKSFEEFKAQFAKAGATRFGSGWAWLCVHKGGKLEVCSTPNQDSPLMPDTACGGFPILGMDVWEHAYYLNYQNRRPDYIEAFFNVINWTEVARLYATEK

SEQ ID NO: 62 - protein, 135 aa, Fusarium solani
MSVGTYSLPALPYAYDALEPSISAQIMELHHSKHHQAYVNNLNAALKTYASATSSNDIAGQIALQSAIKFNGGGHINHSLFWENLSPSSSPDSKPDSAPTLVAEISKTWGSLEAFQETFKKTLLGLQGSGWGWLV

SEQ ID NO: 63 - protein, 212 aa, Fusarium sp.
MSVGTYSLPALPYAYDALEPSISAQIMELHHSKHHQAYVNNLNAALKTYASATSSNDIAGQIALQSAIKFNGGGHINHSLFWENLSPSSSLDSKPDSAPTLVAEISKTWGSIEAFKETFKKTLLGLQGSGWGWLVKDTHGLRIVTTKDQDPVVGGEVPIFGVDMWEHAYYLQYLNGKAAYVDNIWNVINWKTSEARFVGTREDAFKVLRASI

SEQ ID NO: 64 - protein, 206 aa, Klebsiella pneumoniae; identical to SEQ ID NO: 59

SEQ ID NO: 65 - protein, 206 aa, Kocuria palustris
MAEKFVLPDLPYDFAALEPHISGRIMELHHDKHHNTYVTAANKALESMEEARANGDAAGAAKLSKDLQFNLGGHINHSIFWKNMSPDGGDKPTGELASAIDDAFGSFDNFQAQFTAVATTIQGSGWAILAYEPIAGNLVIEQMYDQQNGVPVATFPLLQLDMWEHAFYLDYQNVKPDYVKAWWNVVNWEDVAQRLEKARAGASSLV

SEQ ID NO: 66 - protein, 205 aa, Leucobacter sp.
MTAYSLPELPYDFAALEPHISGKIMELHHGKHHQAYVTGANTALEQLAEARDSGNLGPVNKLEKDLAFNVGGHVNHTIFWNNMSPDGGGKPEGELASAIDEFFGDFEKFQAHFAATAMGVQGSGWAVLAWDTLGQRANIVQMFDQQGNLPAGTVPLLMLDCWEHAYYLDYLNVRADYVKAFWNVANWQDVAKRLDTARSQTPGLI

SEQ ID NO: 67 - protein, 201 aa, Lysinibacillus fusiformis
MAYELPQLTYAYDALEPHIDAKTMEIHHSKHHNTYVTNLNAAVEGTEFAEKNINDLIANLDALPADKQTAVRNNGGGHANHTLFWEVIAPGGSNTPVGEVAKAIDAKFGSFDAFKEEFAKAATTRFGSGWAWLIVDGDSVAVTSTPNQDSPVMEGKTPILGLDVWEHAYYLNYQNRRPDYIGSFWNVVNWDVVEAKFQAAK

SEQ ID NO: 68 - protein, 201 aa, Lysinibacillus sphaericus
MAYELPQLTYAYDALEPHIDAKTMEIHHSKHHNTYVTNLNAAVEGTEFAEKDINDLIANLDALPADKQTAVRNNGGGHANHTLFWEVIAPGGSNTPVGEVAKAIDAKFGSFDAFKEEFAKAATTRFGSGWAWLIVDGDSVAVTSTPNQDSPVMEGKKPILGLDVWEHAYYLNYQNRRPDYIGAFWNVVNWDVVEAKFQAAK

SEQ ID NO: 69 - protein, 201 aa, Lysinibacillus xylanilyticus
MAFELPQLTYAYDALEPHIDAKTMEIHHSKHHNTYVTNLNAAVEGTEFAEKDINDLIANIDALPADKQTAVRNNGGGHANHTLFWELIAPGGSNTPVGEVAKAIDAKFGSFDAFKEEFAKAATTRFGSGWAWLIVDGDSVAVTSTPNQDSPVMEGKTPVLGLDVWEHAYYLNYQNRRPDYIGAFWNVVNWDVVEAKFQAAK

SEQ ID NO: 70 - protein, 208 aa, Microbacterium paraoxydans
MATYTLPDLPYDFAALEPHISGKIMELHHDKHHATYVAGANTALEQLAEARESGNLANVNKLEKDLAFNLGGHVNHSIFWTNLSPNGGGQPEGELKAAIDEYFGSFEKFQAHFTAAATGIQGSGWAVLSWDSIGSRLIIQQLFDQQSNAAQGTIPLFQLDMWEHAFYLDYLNVKADYVKAAWNIANWENVAQRLEVARQQTNGLLVLS

SEQ ID NO: 71 - protein, 208 aa, Micrococcus luteus
MTEYTLPELDYDYAALEPHISARIMELHHSKHHATYVKGANTALEKLAAAREAGDFGSVNQLSKDLAFNLGGHTNHSIFWKNLSPEGGDKPTGELAAAIDEYFGSFDQFQAHFTAAALGIQGSGWAVLAYEPIGGNLVIEQFYDQQNGVPVATIPLFQLDMWEHAFYLDYQNVKADYVKAIWNIVNWADVQARFEAARAGASGLILPA

SEQ ID NO: 72 - protein, 208 aa, Micrococcus lylae
MAEFTLPELDYDYAALEPHISARIMELHHSKHHATYVKGANTALEKLAAAREAEDFGAINQLSKDLAFNLGGHTNHSIFWKNLSPDGGDKPTGELAAAIDEYFGSFEKFQAHFTAAALGIQGSGWAVLAYEPVGGNLVIEQFYDQQNGVPVATIPLFQLDMWEHAFYLDYQNVKADYVKAVWNIVNWEDVQARFEAARNGASGLVLPA

SEQ ID NO: 73 - protein, 208 aa, Micrococcus sp.; identical to SEQ ID NO: 71

SEQ ID NO: 74 - protein, 205 aa, Moraxella sp.
MSYTLPELGYSYDALEPHFDKETMEIHHSRHHQAYVNNANAALANLPEWADKSAEELIANLDKIPEASRTALRNNAGGHANHSLFWTVLKTGTTLGGTLKDAIVRDFGSVEAFQEQFEKAAATRFGSGWAWLVKQGDKLAVVSTANQDSPLMGVAVAGCEGTPIIGLDVWEHAYYLKYQNKRPDYIKAFWNVVNWDEAQRRFDEA

SEQ ID NO: 75 - protein, 225 aa, Mucor circinelloides
MLSVATKSFKHAALKTTAYRSLVAVRSKATLPQLPYGYDELEPVISKDIMETHHAKHHQTYVNNFNVAEEKFEAAVKANDLAQQLALQNALKFNGGGHLNHSIYWENLAPKSQGGGELPKGALLSAIQKEFGSMDSFIEKFNAVAAGVQGSGWCWLGYNKAAQRVEIATSGNQDILQGLVPLLVIDVWEHAYYLQYKNVRAEYLKKVWDVVNWKTVAERFAKAQH

SEQ ID NO: 76 - protein, 198 aa, Nesiobacter exalbescens
MSFKLEDLPYAYDALAPYMSAETLEYHHDKHHQAYVTNGNKLLEGSGLEGKSLEEVVKESYGTNAGLFNNAGQHYNHLHFWKWMKPNGGGALPAALAKKIEEDLGGYDKFRADFINAGVTQFGSGWAWLAVKDGKLVVMKTPNGENPLVHGATPILGCDVWEHSYYIDYRNARPKYLEAFVDNLVNWEYVEELFNKAI

SEQ ID NO: 77 - protein, 207 aa, Nocardia asteroides
MAVYTLPELDWDFSALEPHISGQINEIHFTKHHAAYVAGANAALEKLEAAREAGDHAAIFLNEKNLAFHLGGHTNHSIWWKNLSPNGGDKPVGDLAAAIDDQFGSFDKFRAQFTAAANGLQGSGWAVLGYDTLGQKLLTFQLYDQQANVPIGIIPLLQVDMWEHAFYLQYKNVKADYVTAFWNVVNWADVQDRFARAVTQGKGLIFG

SEQ ID NO: 78 - protein, 198 aa, Ochrobactrum intermedium
MAFELPALPYDYDALAPFMSRETLELHHDKHHQAYVTNGNKLLEGSGLEGKSLEEIVKESYGKNQGLFNNAGQHYNHIQFWKWMKKGGGGKKLPGKLEKAIESDLGGYDKFREDFIAAGVGQFGSGWAWLSVKNGKLEISKTPNGENPLVHGATPILGVDVWEHSYYVDYRNLRPKYLEAFVDNLINWEFVEELYEKA

SEQ ID NO: 79 - protein, 198 aa, Ochrobactrum oryzae; identical to SEQ ID NO: 78

SEQ ID NO: 80 - protein, 204 aa, Paenibacillus macerans
MAHELPALPYPANALEPYIDEQTMNIHHDRHHNTYVTNLNAALESAPALQNKSVEELISDLGSVPENIRTAVRNNGGGHANHSLFWTVIGPGGGGAPSGAVASAIDSDLGGFDKFKEEFTKAATTRFGSGWAWLVVGKDGKLAVTSTPNQDNPLSEGQTPILGLDVWEHAYYLKYQNKRPDYIAAFWNIINWDEVNRRYEAAKK

SEQ ID NO: 81 - protein, 204 aa, Paenibacillus sp.
MAHQLPALPYANNALEPHIDEMTMMIHHDRHHNAYVTNLNAALEGHADLQSKSIEELISDLNSVPEAIRTAVRNNGGGHANHSLFWETIGPNGGGAPTGKLADAIQAELGGFDKFKEDFAKAGATRFGSGWAFLAVTKEGKLKVYSLPNQDSPIMEGETPILGLDVWEHAYYLKYQNKRPDYIAAFWNVVNWEEVGKRYEAAVN

SEQ ID NO: 82 - protein, 205 aa, Pantoea sp.
MSYSLPSLPYAYDALEPHFDKQTMEIHHTKHHQTYVNNANAALEGTEFADLPVDELITKLDQVPADKKTVLRNNAGGHANHSLFWKGLKTGTTLQGDLKAAIEKDFGSVEKFQEEFEKAAATRFGSGWAWLVKKGDKLAVVSTANQDNPLMGEAISGASGYPILGLDVWEHAYYLKYQNKRPDYIKAFWNVVNWDEAAARFASAK

SEQ ID NO: 83 - protein, 228 aa, Penicillium chrysogenum
MAALLSNSARVALRSGASATSKAGAAGLTFARGKATLPDLSYDYGALEPSISGKIMEVHHKNHHNTYVTSYNNTLEQLQEAQAKGDIATQIALKPAVNFHGGGHLNHTLFWENLAPKSAGGGEPPSGALSKAIDSTYGSLGEFQSKMNAALAGIQGSGWAWLVKDKQTGQIGIRTYANQDPVVGQFQPLLGIDAWEHAYYLQYQNRKAEYFSAIWDVINWKTVEKRFT

SEQ ID NO: 84 - protein, 228 aa, Penicillium oxalicum
MAALLANSARTALRSGASASFKSGAAGLTFARGKATLPDLSFDYGALEPSISGKIMEIHHKNHHNTYVTNYNNALEQLQEAQAKNDISAQIALKPAINFNGGGHLNHSLFWENLAPKSAGGGEPPSGSLAQAIDGTYGSFDEFKNKFNTALAGIQGSGWAWLVKDKQTGQIGIRTYANQDPVVGQYQPLLGVDAWEHAYYLQYQNRKAEYFKAIWEVINWKAVEKRFT

SEQ ID NO: 85 - protein, 220 aa, Pleurotus ostreatus
MFAIAKTALRPATARYSVIAAACRSKHTLPDLPYDYNALEPFISEEIMKLHHQKHHQTYVNGLNAAEESYASAQSTKEQIKLQSALKFNGGGHINHSLFWKNLAPASGDGGKLPDGPLKQAIERDFGSVEEFKTKFNTTTAAVQGSGWGWLGLNPTTKKLEVTTTPNQDPLITHAPIIGVDIWEHAFYLQYKNVKPDYLNAIWNVINFKEAEARFLEASA

SEQ ID NO: 86 - protein, 203 aa, Pseudomonas aeruginosa
MPHTLPALPYAYDALEPHIDAQTMEIHHTRHHQTYVNGLNAAVEGTEWAEWPVEKLVAAVGDLPERLRGAVTNHGGGHANHSLFWTVMSPQGGGEPTGELAQAITTQLGGFDAFKEAFTKAALTRFGSGWAWLSVTPQKTLVVESSGNQDSPLMHGNTPILGLDVWEHAYYLKYQNRRPEYIGAFYNVIDWAQVQHRYLEALK

SEQ ID NO: 87 - protein, 203 aa, Pseudomonas azotoformans
MTYTLPALPYAYDALEPHIDAQTMEIHYTKHHQTYINNLNAAVEGTEFAGWPVEKLVSSVETLPEKLRAAVVNQGGGHANHSLFWAVMSPTGGGKPEGALGKAIDEQLGGFDSFKEAFTKAALTRFGSGWAWLSVTPQKTLVVESSGNQDSPLMSGNTPVLGLDVWEHAYYLQYQNRRPEYINAFYSVINWPEVAARYQAALA

SEQ ID NO: 88 - protein, 203 aa, Pseudomonas chlororaphis
MSFTLPALPYAYDALEPHIDAQTMEIHYTKHHQTYINNLNAAVEGTEWADWSVEKLVSGVQQLPEKLRATVINQGGGHANHSLFWAVMAPAGGGKPDGVLGQAIDEQLGGFESFKEAFTKAALTRFGSGWAWLSVTPQKTLVVESSGNQDSPLMNGNTPILGLDVWEHAYYLRYQNRRPEYINAFYNVINWPEVAQRYQAALV

SEQ ID NO: 89 - protein, 208 aa, Pseudomonas citronellolis
MPHSLPALPYAYDALEPHIDARTMEIHHSKHHQTYVNNLNAALEGTPFAELPVEELLQQLNRLPEKLLGAVINNGGGHANHSLFWQVMSPSGGGEPDGALARAIETELGGYAAFRDAFTKAALTRFGSGWAWLCVDSQGRLKVESSANQDSPLMQDLIPILGLDVWEHAYYLQYQNRRPEYIAAFYEVVDWPEVARRYRLALEQRGLA

SEQ ID NO: 90 - protein, 198 aa, Pseudomonas fluorescens
MAFELPPLPYAHDALQPHISKETLEFHHDKHHNTYVVNLNNLVPGTEFEGKTLEEIVKTSSGGIFNNAAQVWNHTFYWNCLAPNAGGQPTGALADAINAAFGSFDKFKEEFSKTSIGTFGSGWGWLVKKADGSLALASTIGAGNPLTSGDTPLLTCDVWEHAYYIDYRNVRPKYVEAFWNLVNWKFVAEQFEGKPFTA

SEQ ID NO: 91 - protein, 198 aa, Pseudomonas monteilii
MAFELPPLPYAHDALQPHISKETLEFHHDKHHNTYVVNLNNLVPGTEFEGKTLEEIVKTSSGGIFNNAAQVWNHTFYWNCLAPNAGGQPTGALADAINAAFGSFDKFKEEFTKTSVGTFGSGWGWLVKKADGSLALASTIGAGCPLTSGDTPLLTCDVWEHAYYIDYRNLRPKYVEAFWNLVNWAFVAEQFEGKTFKA

SEQ ID NO: 92 - protein, 198 aa, Pseudomonas protegens
MAFELPPLPYAHDALQPHISKETLEYHHDKHHNTYVVNLNNLVPGTEFEGKTLEQIVKTSSGGIFNNAAQVWNHTFYWNCLAPNAGGQPTGALAEAINAAFGSFDKFKEEFTKTSVGTFGSGWGWLVKKADGSLALASTIGAGNPLTSGDTPLLTCDVWEHAYYIDYRNVRPKYVEAFWNLVNWKFVAEQFEGKNFVA

SEQ ID NO: 93 - protein, 198 aa, Pseudomonas putida
MAFELPPLPYAHDALQPHISKETLEFHHDKHHNTYVVNLNNLVPGTEFEGKTLEEIVKTSSGGIFNNAAQVWNHTFYWNCLAPNAGGQPTGALAEAINAAFGSFDKFKEEFSKTSIGTFGSGWGWLVKKADGSLALASTIGAGNPLTSGDTPLLTCDVWEHAYYIDYRNLRPKYVEAFWNLVNWKFVAEQFEGKTFTA

SEQ ID NO: 94 - protein, 198 aa, Pseudomonas sp.
MAFELPPLPYAHDALQPHISKETLEFHHDKHHNTYVVNLNNLVPGTEFEGKTLEEIVKTSSGGIFNNAAQVWNHTFYWNCLAPNAGGQPTGALADAINAAFGSFDKFKEEFTKTSVGTFGSGWGWLVKKADGSLALASTIGAGNPLTNGDTPLLTCDVWEHAYYIDYRNLRPKYVEAFWNLVNWKFVAEQFEGKTFTA

SEQ ID NO: 95 - protein, 193 aa, Pseudomonas stutzeri
MAFELPPLPYEKNALEPHMSAETLEFHHDKHHAAYVTNLNNLIQGTEFEGKDLESIIKTSSGGIFNNAAQVWNHTFFWNCLAPNAGGQPTGALADAINASFGSFDKFKEEFTKTAIGTFGSGWAWLVKKSDGSLGLASTIGAGNPMTAGDKPLLTCDVWEHAYYIDYRNARPKFVEAFWNLVNWDFVAKNFAG

SEQ ID NO: 96 - protein, 193 aa, Pseudomonas syringae
MAFELPPLPYAKDALVPHISAETLEYHHDKHHNTYVVNLNNLVPGTEFEGKTLEEIIKTSSGGIFNNAAQVWNHTFYWECLAPNAGGEPTGALADAINAAFGSFEKFKEEFSKTSIGTFGSGWGWLVKKADGSLALASTIGAGNPLTSGDKPLLTCDVWEHAYYIDYRNLRPKYVEAFWNLVNWDFVAKQYAA

SEQ ID NO: 97 - protein, 206 aa, Rahnella aquatilis
MSYSLPSLPYAYDALEPHFDKETMEIHHTKHHQTYVNNANAALESLPELAALPVEELITKLDQVPADKKVVLRNNAGGHANHSLFWKGLKTGTTLQGELKSAIERDFGSVDAFKEAFEKAAATRFGSGWAWLVLKDGKLAVVSTANQDSPLMGEAISGASGYPLLGLDVWEHAYYLKYQNKRPDYIKAFWAVVNWDEAAKRLAEAK

SEQ ID NO: 98 - protein, 192 aa, Ralstonia sp.
MEHTLPPLPYAQDALAPHISKETLEFHYGKHHQTYVTNLNNLIKGTEFENLSLEDIVKKSSGGVFNNAAQVWNHTFYWNGLKPQGGGAPTGALADAINAKWGSFDKFKEEFTKTAIGTFGSGWAWLVKKADGSLDLVSTSNAATPLTTDAKPLLTCDVWEHAYYIDYRNARPKYVEAFWNLVNWDFVAKNFA

SEQ ID NO: 99 - protein, 207 aa, Rhodococcus erythropolis
MSVYTLPELPYDYAALEPHISGKIMELHHDKHHAAYVAGANAALEKLAEARENDTIAAQANLLEKNLAFHLGGHTNHTVFWNNLSADGGDKPEGELAAAIDDNFGSFDLFRAHFSANANAIQGSGWSILAWDSIGQRLIIVQLYDQQGNISIGLTPLLLLDMWEHAFYLDYQNVKGDYVKAFWNIVNWNDVSARFDRARTQTAGLIV

SEQ ID NO: 100 - protein, 207 aa, Rhodococcus rhodochrous; identical to SEQ ID NO: 10

SEQ ID NO: 101 - protein, 207 aa, Rhodococcus sp.
MAEYTLPDLPYDYAALEPHISAKIMELHHSKHHATYVAGANTALEKMAEARENGTMAAQATKLEKDLAFHLGGHTNHSVFWKNLSPEGGDKPDGELGAAIDDAFGSFDAFRDHFSANANSIQGSGWSILAWDSIGQRLIIVQLYDQQGNIPIGLTPLLMLDMWEHAFYLQYQNVKADYVKAFWNIVNWADVQARFDRARTQTAGLLV

SEQ ID NO: 102 - protein, 206 aa, Serratia marcescens
MSYSLPSLPYAYDALEPHFDKQTMEIHHTKHHQTYVNNANTVLEAYPELAKYSVEELIQDLDKVPADKRTFMRNNAGGHANHSFFWKNLKTGTTLGGDLKAAIERDFGSVEKFQEEFEKAAATRFGSGWAWLVLKDGKLAVVSTANQDSPLMGEAFAGAAGFPIIGLDVWEHAYYLKYQNKRPDYIKAFWNVVNWDEAAARFAAKK

SEQ ID NO: 103 - protein, 202 aa, Sphingobacterium multivorum
MAFELEALPYAADALEPHIDKTTMEIHHDRHHQAYVDNLNKAIAGTDAENLSLLDIIKNVSKYSAAVRNNGGGHYNHSLFWKVLGPNAGGTPTGELADAINATFGSFDELKKQLQAAGATRFGSGWAWLIVGADGKLEVTSTPNQDNPLMDVAEVKGTPILGIDVWEHAYYLKFQNKRPAYLEEIFNVINWDAVAQNYAAAK

SEQ ID NO: 104 - protein, 199 aa, Staphylococcus cohnii
MAFELPNLPYGSDALEPHIDQQTMEIHHDKHHNTYVTKLNAAIEGTDLESKSIEEIVANLDSVPENIQTAVRNNGGGHLNHSLFWELLTPNSEEKGTVVDKIKEQWGSLDAFKEEFADKAAARFGSGWAWLVVNNGNLEIVTTPNQDNPLTEGKTPILGLDVWEHAYYLKYQNKRPEYISAFWNVVNWEKVDELYNAAK

SEQ ID NO: 105 - protein, 199 aa, Staphylococcus epidermidis
MAFELPNLPYAYDALEPHIDKQTMEIHHDKHHNTYVTKLNSAVEGTDLEAKSIEEIVANLDSVPSNIQTAVRNNGGGHLNHSLFWELLSPNSEEKGEVVDKIKEQWGSLDEFKKEFADKAAARFGSGWAWLVVNNGQLEIVTTPNQDNPITEGKTPILGLDVWEHAYYLKYQNKRPDYINAFWNVVNWEKVNELYNATK

SEQ ID NO: 106 - protein, 199 aa, Staphylococcus sp.; identical to SEQ ID NO: 105

SEQ ID NO: 107 - protein, 199 aa, Staphylococcus xylosus
MAFELPNLPYGFDALEPHIDQQTMEIHHGKHHNTYVTKLNAAVEGTDLESKSIEEIVANLDSVPENIQTAVRNNGGGHLNHSLFWELLTPNSEEKGTVVDKIKEQWGSLDAFKEEFADKAAARFGSGWAWLVVNNGNLEIVTTPNQDNPITEGKTPILGLDVWEHAYYLKYQNKRPDYISAFWNVVNWEKVDELYNAAK

SEQ ID NO: 108 - protein, 203 aa, Stenotrophomonas humi
MAYTLPKLPYAYDALEPNIDAETMEIHHSKHHQTYINNVNAALEGTEYADLPVEELVKKTKSLPENLQGPVRNNGGGHANHSLFWTVMSPNGGGQPVGDVAKAIDSQLGGFEKFKEAFTKAALTRFGSGWAWLSVTPDKKVVVESSANQDSPLMDGNTPILALDVWEHAYYLKYQNRRPEYIGAFFNVVDWNEVERRYQEAIA

SEQ ID NO: 109 - protein, 203 aa, Stenotrophomonas maltophila
MAYTLPKLSYAYDALEPHIDAATMEIHHTKHHQTYINNVNAALEGTEYADLPVEELVKKLKSLPENLQGPVRNNGGGHANHSLFWTVMAPNAGGNPVGDVAKAIDKDLGGFDKFKDAFTKAALTRFGSGWAWLSVTPDKKVVVESTGNQDSPLMEGNTPILGLDVWEHAYYLKYQNRRPEYIGAFFNVIDWNEVERRYQEAIA

SEQ ID NO: 110 - protein, 203 aa, Stenotrophomonas panacihumi
MAYTLPKLSYAYDALEPNIDAQTMEIHHTKHHQTYINNVNAALEGTEYADLPVEELVSRLKSLPENLQGPVRNNGGGHANHSLFWTVMSPKGGGEPVGEVAKAIDRDVGGFEKFKEAFTKAAISRFGSGWAWLSVTPDKKVVVESTANQDSPLMEGNTPILGLDVWEHAYYLKYQNRRPEYIGAFYNVVDWNEVERRYQAAIA

SEQ ID NO: 111 - protein, 203 aa, Stenotrophomonas sp.; identical to SEQ ID NO: 109

SEQ ID NO: 112 - protein, 203 aa, Streptococcus sp.
MAIILPDLPYAYDALEPYIDTETMKLHHDKHHATYVANVNAALEKHPEIGENLEVLLADVEQIPADIRPAVINNGGGHLNHALFWELLSPEKTEPSAELVQAIDEAFGSFEDFKAAFTAAATTRFGSGWAWLVLNGDGRLEVISTANQDSPIMEGKTPILALDVWEHAYYLNYRNVRPDYIKAFFEVINWPKVAELYKQAKSV

SEQ ID NO: 113 - protein, 213 aa, Streptomyces badius
MATYTLPELPYDYAALEPVINPQIIELHHDKHHAAYVKGANDTLEQLEEARDKEAWGAINGLQKNLAFHLSGHILHSIYWHNMTGDGGGEPLAADGVGDLADAITESFGSYAGFKSQLTKAAATTQGSGWGVLAYEPVSGKLIVEQVYDHQGNVGQGSVPILVFDAWEHAFYLQYKNQKVDFIEAMWQVVNWQDVAKRYAAAKERVNVLLLAP

SEQ ID NO: 114 - protein, 213 aa, Streptomyces griseus; identical to SEQ ID NO: 113

SEQ ID NO: 115 - protein, 213 aa, Streptomyces sp.; identical to SEQ ID NO: 113

SEQ ID NO: 116 - protein, 258 aa, Trichoderma harzianum
MASRVILLTALSLLPLGCYSSSPDPLKPEFASVKIQEPLTDSFEPTMSIGTFSLPALPYAYDALQPSISREIMELHHSKHHQAYVTNLNNALKTYSTAITANDVPSQIALQSAIKFNGGGHINHSLFWKNLSPSSSADANPASAPTLTAEIDKTWGSLDGFKEAMGKALLGLQGSGWGWLVKDGNTLRIVTTKDQDPVVGGEVPILGIDMWEHAYYLQYYNGKAAYVENIWKVINWATAEERFKGDREDAFKILKAAL

SEQ ID NO: 117 - protein, 231 aa, Trichoderma virens
MSSSLLRTTPLLRAGLRARAAKPLAAMATTSFTRGKATLPDLPYDYGALEPYISGQIMELHHSKHHQTYVNGFNAAVEALAEAEAKGDSKAAAAQAPLLNFHGGGHVNHSLFWENLAPNGKGGGGEPEGKLLTAINQDFGSFENLKKQTNTALAGIQGSGWAWLVKDKSSGNLCLVTRANQDPVTGNFEPLLGIDAWEHAYYLQYQNRKAEYFSAIWDVINWGTVSKRFGN

SEQ ID NO: 118 - protein, 194 aa, Vibrio alginolyticus
MAFELPALPYAKDALEPHISAETLDYHHGKHHNTYVVKLNGLIPGTEFEGKTLEEIIKTSTGGVFNNAAQIWNHTFYWHCLAPNAGGEPTGAVADAINAAFGSFEEFKAKFTDSAINNFGSSWTWLVKKADGSLEIVNTSNAATPLTEEGTTPLLTVDLWEHAYYIDYRNVRPDYMNGFWALVNWDFVAENLAK

SEQ ID NO: 119 - protein, 194 aa, Vibrio parahaemolyticus; identical to SEQ ID NO: 118

SEQ ID NO: 120 - protein, 202 aa, Brucella anthropi
MSFTLPPLPFATNALEGVGMGTETLELHHGKHHQAYVTALNGFVEKNDALKGKSLEEIVLFAKDKADLAPVFNNAGQHWNHDLFWQNLSATGGRLPGALEKKIVEDFGSVDQFKEAFKAAAVGQFGSGWAWLVLANDGKLKVTKTPNGSNPLATGEGKALLGLDVWEHSYYLDFRNRRPDYVTNFLDKLANYEFAEATLKAA

SEQ ID NO: 121 - protein, 198 aa, Brucella anthropi
MAFELPALPYDYDALAPFMSRETLELHHDKHHQAYVTNGNKLLEGSGLEGKSLEEIVKESYGKNQGLFNNAGQHYNHILFWKWMKKNGGGKKLPGKLEKAIESDLGGYDKFRADFIAAGVGQFGSGWAWLSVKDGKLEISKTPNGENPLVHGATPILGVDVWEHSYYVDYRNLRPKYLEAFVDSLINWEFVEELYEKA

SEQ ID NO: 122 - protein, 203 aa, Halomonas venusta
MAHTLPELPYAYDALEPHIDAMTMEIHHSRHHNTYVTSLNAALEGTGLEDVPVEELVANLDRVPEEKRQAVINHGGGHANHSMFWQMMSPNGGGQPQGDVAKAIEAELGGLDAFKEAFKAAALGRFGSGWAWLSVTPEKKLVVENTLNQDSPLMHGNTPVLGLDVWEHAYYLKFQNKRPDYVAAFFNVVNWEDVERRYQAAIA

SEQ ID NO: 123 - protein, 202 aa, Exiguobacterium sp.
MAKFELPQLDYAYDALEPHIDARTMEIHHTKHHNTYVTNVNNALEGKDVEYSSLEDLLQKIDSLPADIQTAVRNNGGGHWNHSFFWKLLKKNENGTPSGDLAAAIDESFGSFDAFKDEFTKAATTRFGSGWAWLVVKNGKLEVTSTPNQDSPIMEGVTPILGLDVWEHAYYLNYQNRRPDYINAFFHVIDWDYVAKLYADAK

SEQ ID NO: 124 - protein, 559 aa, Pseudomonas sp.
MSNKNSDDLNRQASENTLGLNPVIGLRGKDLLTSARMVLTQAIKQPIHSVKHVAHFGIELKNVMFGKSKLQPESDDRRFNDPAWSQNPLYKRYLQTYLAWRKELHDWIGNSKLSEQDINRAHFVITLMTEAMAPTNSAANPAAVKRFFETGGKSLLDGLTHLAKDLVNNGGMPSQVDMGAFEVGKSLGTTEGAVVFRNDVLELIQYRPTTEQVHERPLLVVPPQINKFYVFDLSPDKSLARFCLSNNQQTFIVSWRNPTKAQREWGLSTYIDALKEAVDVVSAITGSKDINMLGACSGGITCTALLGHYAALGEKKVNALTLLVSVLDTTLDSQVALFVDEKTLEAAKRHSYQAGVLEGRDMAKVFAWMRPNDLIWNYWVNNYLLGNEPPVFDILFWNNDTTRLPAAFHGDLIEMFKNNPLVRANALEVSGTPIDLKQVTADIYSLAGTNDHITPWKSCYKSAQLFGGKVEFVLSSSGHIQSILNPPGNPKSRYMTSTDMPATANEWQENSTKHTDSWWLHWQAWQAERSGKLKKSPTSLGNKAYPSGEAAPGTYVHER

SEQ ID NO: 125 - DNA, 1680 bp, synthetic construct
  source 1..1680: mol_type = other DNA
atgtccaacaagaactccgatgacctcaaccgacaggcctctgagaacactctcggcctgaaccctgtcatcggtctgcgaggaaaggacctgctgacttctgctcgaatggtcctgacccaggccatcaagcagcctatccattcggtgaagcacgtcgctcacttcggcatcgagctcaagaacgtgatgttcggcaagtctaagctgcagcctgagtctgatgaccgacgattcaacgatcccgcttggtctcagaaccccctctacaagcgatacctgcagacctacctggcttggcgaaaggagctgcatgactggatcggaaactccaagctgtctgagcaggacattaaccgagcccacttcgtgattaccctcatgactgaggctatggcccccactaactctgctgccaaccccgctgccgttaagcgattcttcgagaccggtggcaagtctctgctcgacggactgacccatctggctaaggatctggtgaacaacggtggcatgccctcgcaggtcgatatgggagccttcgaggttggaaagtccctgggcaccactgagggagccgtggtcttccgaaacgacgtgctggagctcatccagtaccgacccactaccgagcaggtgcatgagcgacccctgctcgtggtccctccccagattaacaagttctacgtgtttgacctctcccctgataagtctctggcccgattctgtctgtctaacaaccagcagaccttcatcgtttcgtggcgaaaccctaccaaggctcagcgagagtggggtctgtcgacttacatcgatgctctgaaggaagctgtcgatgtggtttcggctatcaccggttcgaaggacatcaacatgctgggcgcttgttctggcggaatcacctgcaccgctctcctgggtcattacgctgccctgggagagaagaaggttaacgctctgactctcctggtgtctgttctggacaccactctggactctcaggtcgctctgttcgtcgacgagaagactctcgaggccgctaagcgacactcttaccaggctggagtgctggagggtcgagatatggccaaggtttttgcctggatgcgacccaacgacctcatttggaactactgggtgaacaactacctcctgggcaacgagccccctgtttttgacatcctgttctggaacaacgacaccactcgactgcctgctgcctttcatggtgacctgatcgagatgttcaagaacaaccccctggtgcgagctaacgccctggaggtttctggaactcccatcgacctgaagcaggtcactgccgatatctactctctggctggaactaacgaccatatcaccccttggaagtcctgctacaagtctgcccagctctttggtggaaaggtcgagttcgttctctcgtcttccggtcacattcagtcgatcctgaaccctcccggtaaccccaagtctcgatacatgacttcgaccgacatgcccgccactgctaacgagtggcaggagaactccactaagcacaccgattcttggtggctgcattggcaggcttggcaggccgagcgatctggaaagctcaagaagtcgcctacctctctgggtaacaaggcctacccctctggcgaggccgctcccggaacctacgtgcacgagcgataa

SEQ ID NO: 126 - DNA, 35 bp, synthetic construct
  misc_feature 1..35: note = oPlastBug-266 oligonucleotide
  source 1..35: mol_type = other DNA
caacggaatgcgtgcgccggtgaccttggtggttc

SEQ ID NO: 127 - DNA, 37 bp, synthetic construct
  misc_feature 1..37: note = oPlastBug-267 oligonucleotide
  source 1..37: mol_type = other DNA
cttgttggacattttaattaaggaagctgatctggtg

SEQ ID NO: 128 - DNA, 28 bp, synthetic construct
  misc_feature 1..28: note = oPlastBug-268 oligonucleotide
  source 1..28: mol_type = other DNA
ccttaattaaaatgtccaacaagaactc

SEQ ID NO: 129 - DNA, 47 bp, synthetic construct
  misc_feature 1..47: note = oPlastBug-270 oligonucleotide
  source 1..47: mol_type = other DNA
gaacagaaggaatgcacgcgttaattaattatcgctcgtgcacgtag

SEQ ID NO: 130 - protein, 302 aa, Bacillus cereus
MKLSTILFTACATIAAALPSPITPSEAAVLQKRAFVTVEKDVHIFVQDVNPGPGSKTVFFVHGWPLNHQMYQYQLNVLPEHGFRCIAMDIRGNGQSDKPWTGYTYDRLADDIAIVLEALQVENATLVGFSVGGALSIRYMSRYNGRRISKLALIDAVSPSFVKNQESPYGVPKEQADALINQMYANLPKFLNDVSLSFFNRNLGAATLEWFSYLGMQSASYALIKILQAAANEDVTKDLNKINVPTKIFHGVHDQLIPYKSAELTQQRIKNSQLHALTNSGHGSPIDQADELNKELIKFLHA

SEQ ID NO: 131 - DNA, 909 bp, synthetic construct
  source 1..909: mol_type = other DNA
atgaagctctccaccatcctgttcaccgcttgtgccaccattgccgctgccctcccttcccccattactccttcggaggccgctgtgctccagaagcgagcctttgtgaccgtcgagaaggacgtgcacattttcgtgcaggacgttaaccctggccctggatctaagaccgtctttttcgtgcacggttggcctctgaaccaccagatgtaccagtaccagctcaacgttctgcctgagcatggattccgatgcatcgctatggacatccgaggaaacggacagtctgacaagccctggactggttacacctacgaccgactggccgatgacatcgctatcgtgctggaggctctgcaggtcgagaacgctactctggtgggtttttccgttggcggagctctgtctattcgatacatgtcccgatacaacggtcgacgaatctctaagctggctctcatcgatgccgtctctccctccttcgtgaagaaccaggagtccccttacggtgttcccaaggagcaggccgacgctctgatcaaccagatgtacgctaacctgcctaagttcctgaacgatgtctccctgtctttcttcaaccgaaacctcggagccgctactctggagtggttttcctacctgggaatgcagtcggcttcctacgccctgatcaagatcctgcaggctgctgccaacgaggatgtcaccaaggacctgaacaagatcaacgtgcccaccaagatttttcacggcgtgcacgatcagctgattccctacaagtctgccgagctgacccagcagcgaatcaagaactcgcagctgcacgccctgaccaactccggtcatggctcccccattgaccaggctgacgagctgaacaaggagctgatcaagtttctgcacgcttga